SEQUENCE LISTING (17 sequences)

SEQ ID NO: 1 - DNA, 1449 bp, Homo sapiens
agaaaggagtggggttgaaaagcgcttgcgcaggacggctacggtacgggggtgggagggcttcggagcacgcgcgcggaggcgggacttgggaagcgctcgcgagatcttcagggtctatatataagcgcggggagcctgcgtcctttccctggtgtgattccgtcctgcgcggttgttctctggagcagcgttcttttatctccgtccgccttctctcctacctaagtgcgtgccgccacccgatggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaatctatacgagatactccagccaaaaatgcacaaaagtcaaatcagaatggaaaagactcaaaaccatcatcaacaccaagatcaaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgcctgtttagtttttaaagatggaactccaccctttgcttggttttaagtatgtatggaatgttatgataggacatagtagtagcggtggtcagacatggaaatggtggggagacaaaaatatacatgtgaaataaaactcagtattttaataaagta

SEQ ID NO: 2 - protein, 294 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 3 - DNA, 4395 bp, Homo sapiens
gcagtgggctctggcggaggtcgggagaactgcagggcgaaggccgccgggggctccgcgggctgcggggggaggcacttgacaccggcccggggagaggaggggccgctgtccctgcggccagtgctggatgcggggacccagcgcagaagcagcgccaggtggagccatcgaagcccccacccacaggctgacagaggcaccgttcaccagagggctcaacaccgggatctatgtttaagttttaactctcgcctccaaagaccacgataattccttccccaaagcccagcagccccccagccccgcgcagccccagcctgcctcccggcgcccagatgcccgccatgccctccagcggccccggggacaccagcagctctgctgcggagcgggaggaggaccgaaaggacggagaggagcaggaggagccgcgtggcaaggaggagcgccaagagcccagcaccacggcacggaaggtggggcggcctgggaggaagcgcaagcaccccccggtggaaagcggtgacacgccaaaggaccctgcggtgatctccaagtccccatccatggcccaggactcaggcgcctcagagctattacccaatggggacttggagaagcggagtgagccccagccagaggaggggagccctgctggggggcagaagggcggggccccagcagagggagagggtgcagctgagaccctgcctgaagcctcaagagcagtggaaaatggctgctgcacccccaaggagggccgaggagcccctgcagaagcgggcaaagaacagaaggagaccaacatcgaatccatgaaaatggagggctcccggggccggctgcggggtggcttgggctgggagtccagcctccgtcagcggcccatgccgaggctcaccttccaggcgggggacccctactacatcagcaagcgcaagcgggacgagtggctggcacgctggaaaagggaggctgagaagaaagccaaggtcattgcaggaatgaatgctgtggaagaaaaccaggggcccggggagtctcagaaggtggaggaggccagccctcctgctgtgcagcagcccactgaccccgcatcccccactgtggctaccacgcctgagcccgtggggtccgatgctggggacaagaatgccaccaaagcaggcgatgacgagccagagtacgaggacggccggggctttggcattggggagctggtgtgggggaaactgcggggcttctcctggtggccaggccgcattgtgtcttggtggatgacgggccggagccgagcagctgaaggcacccgctgggtcatgtggttcggagacggcaaattctcagtggtgtgtgttgagaagctgatgccgctgagctcgttttgcagtgcgttccaccaggccacgtacaacaagcagcccatgtaccgcaaagccatctacgaggtcctgcaggtggccagcagccgcgcggggaagctgttcccggtgtgccacgacagcgatgagagtgacactgccaaggccgtggaggtgcagaacaagcccatgattgaatgggccctggggggcttccagccttctggccctaagggcctggagccaccagaagaagagaagaatccctacaaagaagtgtacacggacatgtgggtggaacctgaggcagctgcctacgcaccacctccaccagccaaaaagccccggaagagcacagcggagaagcccaaggtcaaggagattattgatgagcgcacaagagagcggctggtgtacgaggtgcggcagaagtgccggaacattgaggacatctgcatctcctgtgggagcctcaatgttaccctggaacaccccctcttcgttggaggaatgtgccaaaactgcaagaactgctttctggagtgtgcgtaccagtacgacgacgacggctaccagtcctactgcaccatctgctgtgggggccgtgaggtgctcatgtgcggaaacaacaactgctgcaggtgcttttgcgtggagtgtgtggacctcttggtggggccgggggctgcccaggcagccattaaggaagacccctggaactgctacatgtgcgggcacaagggtacctacgggctgctgcggcggcgagaggactggccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtaagggacatgggggcaaactgaggtagcgacacaaagttaaacaaacaaacaaaaaacacaaaacataataaaacaccaagaacatgaggatggagagaagtatcagcacccagaagagaaaaaggaatttaaaacaaaaaccacagaggcggaaataccggagggctttgccttgcgaaaagggttggacatcatctcctgatttttcaatgttattcttcagtcctatttaaaaacaaaaccaagctcccttcccttcctcccccttcccttttttttcggtcagaccttttattttctactcttttcagaggggttttctgtttgtttgggttttgtttcttgctgtgactgaaacaagaaggttattgcagcaaaaatcagtaacaaaaaatagtaacaataccttgcagaggaaaggtgggagagaggaaaaaaggaaattctatagaaatctatatattgggttgtttttttttttgttttttgttttttttttttgggtttttttttttactatatatcttttttttgttgtctctagcctgatcagataggagcacaagcaggggacggaaagagagagacactcaggcggcagcattccctcccagccactgagctgtcgtgccagcaccattcctggtcacgcaaaacagaacccagttagcagcagggagacgagaacaccacacaagacatttttctacagtatttcaggtgcctaccacacaggaaaccttgaagaaaatcagtttctagaagccgctgttacctcttgtttacagtttatatatatatgatagatatgagatatatatataaaaggtactgttaactactgtacaacccgacttcataatggtgctttcaaacagcgagatgagtaaaaacatcagcttccacgttgccttctgcgcaaagggtttcaccaaggatggagaaagggagacagcttgcagatggcgcgttctcacggtgggctcttccccttggtttgtaacgaagtgaaggaggagaacttgggagccaggttctccctgccaaaaagggggctagatgaggtggtcgggcccgtggacagctgagagtgggattcatccagactcatgcaataaccctttgattgttttctaaaaggagactccctcggcaagatggcagagggtacggagtcttcaggcccagtttctcactttagccaattcgagggctccttgtggtgggatcagaactaatccagagtgtgggaaagtgacagtcaaaaccccacctggagcaaataaaaaaacatacaaaacgtactggtgctttcctgt

SEQ ID NO: 4 - protein, 912 aa, Homo sapiens
MPAMPSSGPGDTSSSAAEREEDRKDGEEQEEPRGKEERQEPSTTARKVGRPGRKRKHPPVESGDTPKDPAVISKSPSMAQDSGASELLPNGDLEKRSEPQPEEGSPAGGQKGGAPAEGEGAAETLPEASRAVENGCCTPKEGRGAPAEAGKEQKETNIESMKMEGSRGRLRGGLGWESSLRQRPMPRLTFQAGDPYYISKRKRDEWLARWKREAEKKAKVIAGMNAVEENQGPGESQKVEEASPPAVQQPTDPASPTVATTPEPVGSDAGDKNATKAGDDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPVCHDSDESDTAKAVEVQNKPMIEWALGGFQPSGPKGLEPPEEEKNPYKEVYTDMWVEPEAAAYAPPPPAKKPRKSTAEKPKVKEIIDERTRERLVYEVRQKCRNIEDICISCGSLNVTLEHPLFVGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 5 - DNA, 4745 bp, Homo sapiens
ttttctgcccttctttgctttggtggcttccttgtggttcctcagtggtgcctgcaacccctggttcacctccttccaggttctggctccttccagccatggctctcagagtccttctgttaacagccttgaccttatgtcatgggttcaacttggacactgaaaacgcaatgaccttccaagagaacgcaaggggcttcgggcagagcgtggtccagcttcagggatccagggtggtggttggagccccccaggagatagtggctgccaaccaaaggggcagcctctaccagtgcgactacagcacaggctcatgcgagcccatccgcctgcaggtccccgtggaggccgtgaacatgtccctgggcctgtccctggcagccaccaccagcccccctcagctgctggcctgtggtcccaccgtgcaccagacttgcagtgagaacacgtatgtgaaagggctctgcttcctgtttggatccaacctacggcagcagccccagaagttcccagaggccctccgagggtgtcctcaagaggatagtgacattgccttcttgattgatggctctggtagcatcatcccacatgactttcggcggatgaaggagtttgtctcaactgtgatggagcaattaaaaaagtccaaaaccttgttctctttgatgcagtactctgaagaattccggattcactttaccttcaaagagttccagaacaaccctaacccaagatcactggtgaagccaataacgcagctgcttgggcggacacacacggccacgggcatccgcaaagtggtacgagagctgtttaacatcaccaacggagcccgaaagaatgcctttaagatcctagttgtcatcacggatggagaaaagtttggcgatcccttgggatatgaggatgtcatccctgaggcagacagagagggagtcattcgctacgtcattggggtgggagatgccttccgcagtgagaaatcccgccaagagcttaataccatcgcatccaagccgcctcgtgatcacgtgttccaggtgaataactttgaggctctgaagaccattcagaaccagcttcgggagaagatctttgcgatcgagggtactcagacaggaagtagcagctcctttgagcatgagatgtctcaggaaggcttcagcgctgccatcacctctaatggccccttgctgagcactgtggggagctatgactgggctggtggagtctttctatatacatcaaaggagaaaagcaccttcatcaacatgaccagagtggattcagacatgaatgatgcttacttgggttatgctgccgccatcatcttacggaaccgggtgcaaagcctggttctgggggcacctcgatatcagcacatcggcctggtagcgatgttcaggcagaacactggcatgtgggagtccaacgctaatgtcaagggcacccagatcggcgcctacttcggggcctccctctgctccgtggacgtggacagcaacggcagcaccgacctggtcctcatcggggccccccattactacgagcagacccgagggggccaggtgtccgtgtgccccttgcccagggggcagagggctcggtggcagtgtgatgctgttctctacggggagcagggccaaccctggggccgctttggggcagccctaacagtgctgggggacgtaaatggggacaagctgacggacgtggccattggggccccaggagaggaggacaaccggggtgctgtttacctgtttcacggaacctcaggatctggcatcagcccctcccatagccagcggatagcaggctccaagctctctcccaggctccagtattttggtcagtcactgagtgggggccaggacctcacaatggatggactggtagacctgactgtaggagcccaggggcacgtgctgctgctcaggtcccagccagtactgagagtcaaggcaatcatggagttcaatcccagggaagtggcaaggaatgtatttgagtgtaatgatcaggtggtgaaaggcaaggaagccggagaggtcagagtctgcctccatgtccagaagagcacacgggatcggctaagagaaggacagatccagagtgttgtgacttatgacctggctctggactccggccgcccacattcccgcgccgtcttcaatgagacaaagaacagcacacgcagacagacacaggtcttggggctgacccagacttgtgagaccctgaaactacagttgccgaattgcatcgaggacccagtgagccccattgtgctgcgcctgaacttctctctggtgggaacgccattgtctgctttcgggaacctccggccagtgctggcggaggatgctcagagactcttcacagccttgtttccctttgagaagaattgtggcaatgacaacatctgccaggatgacctcagcatcaccttcagtttcatgagcctggactgcctcgtggtgggtgggccccgggagttcaacgtgacagtgactgtgagaaatgatggtgaggactcctacaggacacaggtcaccttcttcttcccgcttgacctgtcctaccggaaggtgtccacgctccagaaccagcgctcacagcgatcctggcgcctggcctgtgagtctgcctcctccaccgaagtgtctggggccttgaagagcaccagctgcagcataaaccaccccatcttcccggaaaactcagaggtcacctttaatatcacgtttgatgtagactctaaggcttcccttggaaacaaactgctcctcaaggccaatgtgaccagtgagaacaacatgcccagaaccaacaaaaccgaattccaactggagctgccggtgaaatatgctgtctacatggtggtcaccagccatggggtctccactaaatatctcaacttcacggcctcagagaataccagtcgggtcatgcagcatcaatatcaggtcagcaacctggggcagaggagcctccccatcagcctggtgttcttggtgcccgtccggctgaaccagactgtcatatgggaccgcccccaggtcaccttctccgagaacctctcgagtacgtgccacaccaaggagcgcttgccctctcactccgactttctggctgagcttcggaaggcccccgtggtgaactgctccatcgctgtctgccagagaatccagtgtgacatcccgttctttggcatccaggaagaattcaatgctaccctcaaaggcaacctctcgtttgactggtacatcaagacctcgcataaccacctcctgatcgtgagcacagctgagatcttgtttaacgattccgtgttcaccctgctgccgggacagggggcgtttgtgaggtcccagacggagaccaaagtggagccgttcgaggtccccaaccccctgccgctcatcgtgggcagctctgtcgggggactgctgctcctggccctcatcaccgccgcgctgtacaagctcggcttcttcaagcggcaatacaaggacatgatgagtgaagggggtcccccgggggccgaaccccagtagcggctccttcccgacagagctgcctctcggtggccagcaggactctgcccagaccacacgtagcccccaggctgctggacacgtcggacagcgaagtatccccgacaggacgggcttgggcttccatttgtgtgtgtgcaagtgtgtatgtgcgtgtgtgcaagtgtctgtgtgcaagtgtgtgcacatgtgtgcgtgtgcgtgcatgtgcacttgcacgcccatgtgtgagtgtgtgcaagtatgtgagtgtgtccaagtgtgtgtgcgtgtgtccatgtgtgtgcaagtgtgtgcatgtgtgcgagtgtgtgcatgtgtgtgctcaggggcgtgtggctcacgtgtgtgactcagatgtctctggcgtgtgggtaggtgacggcagcgtagcctctccggcagaagggaactgcctgggctcccttgtgcgtgggtgaagccgctgctgggttttcctccgggagaggggacggtcaatcctgtgggtgaagacagagggaaacacagcagcttctctccactgaaagaagtgggacttcccgtcgcctgcgagcctgcggcctgctggagcctgcgcagcttggatggagactccatgagaagccgtgggtggaaccaggaacctcctccacaccagcgctgatgcccaataaagatgcccactgaggaatgatgaagcttcctttctggattcatttattatttcaatgtgactttaattttttggatggataagcttgtctatggtacaaaaatcacaaggcattcaagtgtacagtgaaaagtctccctttccagatattcaagtcacctccttaaaggtagtcaagattgtgttttgaggtttccttcagacagattccaggcgatgtgcaagtgtatgcacgtgtgcacacacaccacacatacacacacacaagcttttttacacaaatggtagcatactttatattggtctgtatcttgctttttttcaccaatatttctcagacatcggttcatattaagacataaattactttttcattcttttataccgctgcatagtattccattgtgtgagtgtaccataatgtatttaaccagtcttcttttgatatactattttcattctcttgttattgcatcaatgctgagttaataaatcaaatatatgtcatttttgcatatatgtaaggataa

SEQ ID NO: 6 - protein, 1153 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGQRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 7 - DNA, 1395 bp, Homo sapiens
gggccgccggctgccagcccagggcggggcggagccctacttctggccgaccgcgtaggcggtgcttgaacttagggctgcttgtggctgggcactcgcgcagaggccggcccgacgagccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgaattcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtatatgaatggtattctgaaatgtgacagaaaaccagacagaaccaagtaagtacactgcaggctgtgcactctgtgccagaacaacagcagccctgctgtagatgcccattacttgggaagccagattgtactcatctgttggttgccttgtacagtaataagaagtaacatcagcagatatactaaccatagtgtattaagtaccaactgtgtgccatgcactgcactagccactctctgcatatcgtttcctggaaccctctcaatgagccttgcaggaggagtatcataccccttgcttagagacagcgcatatgagattcatagaagtcaagtgagatatcaaagtcacgttgctagtaagtgatgggctgggatttaaatctaggtctttggactccaagagttatactgttaaccactacaatacactccctccacctcccatcccagaaaaagttggatcagggatagggttcacttcaggagccagggtcagagctgggcctgtagtcacatataaaagataggacacaaacacaattagcagcagtgttggtttaataaaccgccgta

SEQ ID NO: 8 - protein, 248 aa, Homo sapiens
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTK

SEQ ID NO: 9 - DNA, 25 bp, Artificial sequence
  NPM1 forward primer
gatgtctatgaagtgttgtggttcc

SEQ ID NO: 10 - DNA, 20 bp, Artificial sequence
  NPM1 reverse primer
ggacagccagatatcaactg

SEQ ID NO: 11 - DNA, 20 bp, Artificial sequence
  DNMT3A R882 primer
gacgtctccaacatgacccg

SEQ ID NO: 12 - DNA, 23 bp, Artificial sequence
  DNMT3A R882C primer
tactgacgtctccaacatgacct

SEQ ID NO: 13 - DNA, 21 bp, Artificial sequence
  DNMT3A R882H primer
acgtctccaacatgagccaat

SEQ ID NO: 14 - DNA, 23 bp, Artificial sequence
  DNMT3A R882S primer
tactgacgtctccaacatgaaca

SEQ ID NO: 15 - DNA, 21 bp, Artificial sequence
  DNMT3A R882P primer
acgtctccaacatgagcccat

SEQ ID NO: 16 - DNA, 23 bp, Artificial sequence
  DNMT3A R882G primer
tactgacgtctccaacatgaacg

SEQ ID NO: 17 - DNA, 21 bp, Artificial sequence
  DNMT3A R882 variant universal reverse primer
gtgtcgctacctcagtttgcc

(19) United States
(12) Patent Application Publication
(10) Pub. No.: US 2020/0054639 A1
(43) Pub. Date: Feb. 20, 2020

(54) COMBINATIONS OF AGENTS TO TREAT HEMATOLOGICAL MALIGNANCIES

(71) Applicant: Oregon Health & Science University, Portland, OR (US)

(72) Inventors: Jeffrey Tyner, Portland, OR (US); Stephen Kurtz, Portland, OR (US); Brian Druker, Portland, OR (US); Elie Traer, Portland, OR (US); Christopher Eide, Portland, OR (US); Motomi Mori, Portland, OR (US); Andrew Kaempf, Portland, OR (US)

(73) Assignee: Oregon Health & Science University, Portland, OR (US)

(21) Appl. No.: 16/346,492

(22) PCT Filed: Oct. 31, 2017
(86) PCT No.: PCT/US2017/059404

§ 371 Date: Apr. 30, 2019

Related U.S. Application Data
(60) Provisional application No. 62/415,015, filed on Oct. 31, 2016.

(51) Int. Cl. A61K 31/519; A61K 31/44; A61K 31/47; A61K 31/551; A61K 31/635; A61P 35/02; C12Q 1/6886; C12Q 1/6858; C12Q 1/6827

(52) U.S. Cl. CPC A61K 31/519; A61K 31/44; A61K 31/47; A61K 31/551; A61K 45/06; A61P 35/02; C12Q 1/6886; C12Q 1/6858; C12Q 1/6827; A61K 31/635

(57) ABSTRACT

Methods of treating acute myeloid leukemia, chronic lymphocytic leukemia and myeloproliferative neoplasms that involve the administration of combinations of small molecule compounds are disclosed.

### CROSS-REFERENCE TO RELATED APPLICATION

This application claims priority to U.S. Provisional Patent Application No. 62/415,015 filed Oct. 31, 2016 which is fully incorporated by reference herein.

### FIELD

The field involves methods of treating cancer. More specifically the field involves methods of using small molecule pharmaceutical compositions to treat hematological malignancies.

### BACKGROUND

The promise of precision medicine is the ability to align medical interventions with individual patients at the time of diagnosis and to alter treatment regimens as new mutations arise or as responses diminish. Technical developments in next generation sequencing and computational biology have accelerated the development of precision medicine; however, fundamental challenges remain. Although whole genome and exome sequencing technologies can identify many target mutations, these techniques are analytically intensive and may not reliably detect translocations, zygosity changes, and low-allele burden mutations with clinical significance. A further hindrance to the clinical utility of mutation status is a lack of drug therapies that selectively target cancer-associated mutations; effective drugs exist for only a subset of the genes currently known to underlie tumorigenesis. These limitations invite a complementary strategy that assesses drug sensitivities obtained with targeted agents designed to inhibit discrete cellular processes as a way to identify phenotypic indications for specific cancers (Friedman A A et al, Nat Rev Cancer 15, 747-756 (2015); incorporated by reference herein). Associating phenotypic responses with particular genetic alterations may then reveal precision-based therapies.

Acute Myeloid Leukemia (AML) is a cancer resulting from the enhanced proliferation and impaired differentiation of hematopoietic stem and progenitor cells. AML diagnosis relies on cytogenetic analysis as recurrent chromosomal variations represent established prognostic markers, although nearly half of AML patients have a normal karyotype. As there is significant heterogeneity in this group, it is essential to understand the genetic and epigenetic changes relevant to AML pathogenesis. DNA sequencing of 200 AML patients uncovered an average of 13 somatic mutations in each genome of which 5 mutations were recurrent (The Cancer Geneome Atlas, N Engl J Med 368, 2059-2074 (2013); incorporated by reference herein). Common mutations in AML that are also driver mutations represent potential therapeutic targets. Recurrent mutations in transcription factors and epigenetic regulators identified in AML suggest that aberrant transcriptional circuits are a common feature of leukemogenesis (Dohner H et al, Blood 115, 453-474 (2010); incorporated by reference herein). Collectively, these circuits may drive oncogenic gene expression programs to inhibit differentiation and activate self-renewal, and generate leukemia stem cells (LSCS) responsible for the initiation and propagation of disease (Chao M P et al, Cold Spr Harb Symp Quant Biol 73, 439-449 (2008); Reya T et al, Nature 414, 105-111 (2001); and Somerville T C and Cleary M L, Cancer Cell 10, 257-268 (2006); all of which are incorporated by reference herein. Of the many genes commonly mutated in AML, targeted therapies in clinical trials or clinical use have been developed for only five: PML-RARA, FLT3, KIT, IDH1 and IDH2.

AML is presently treated with chemotherapy consisting of cytarabine and daunorubucin, which is effective in 40% of adults younger than 60 years of age (Dohner H et al, Blood 115, 453-474 (2010); incorporated by reference herein). However, the outcome in older patients, who represent the majority of patients with the disease and are unable to receive intensive chemotherapy, is poor with a median survival of 5 to 10 months. A key exception is the subset of AML patients with acute promyelocytic leukemia (APL), where the use of all-trans retinoic acid (ATRA) therapy results in excellent and durable responses, suggesting the potential value of targeted therapies for other AML subgroups (Ravandi F et al, J Clin Oncol 27, 504-510 (2009) and Lo-Coco F et al, N Engl J Med 369, 1472 (2013); both of which are incorporated by reference herein. Developments in understanding the molecular pathogenesis of AML have resulted in a growing number of molecularly targeted drug candidates. However, several factors hinder the development of effective single-agent targeted treatments, including the intratumoral heterogeneity of hematologic malignancies, the emergence of genetically heterogeneous subclones leading to relapse, and rescue signals from the tumor microenvironment. Attempts to develop small molecule inhibitors of the tyrosine kinase FLT3, in which activating mutations are detected in 30% of adult AML cases, illuminate the difficulty for effective single agent targeted therapies. The short duration of response to FLT3 inhibitors is largely attributable to the rapid selection for and expansion of drug resistant subclones (Man C H et al, Blood 119, 5133-5143 (2012); Shah N P et al, Br J Haematol 162, 548-552 (2013); and Baker S D et al, Clin Cancer Res 19, 5758-5768 (2013); all of which are incorporated by reference herein). Targeted drugs may yet improve treatment outcomes; however, it may be difficult for these compounds, if used as single agents, to produce durable remissions necessary for either long-term disease management or bridging the patient to successful bone marrow transplantation therapy, the only current potential for cure. Combinations that modulate distinct pathways may provide an opportunity for improved responses (Shafer D and Grant S, Blood Rev 30, 275-283 (2016); incorporated by reference herein). Indeed, the combination of a MEK inhibitor (trametinib) with a RAF inhibitor (dabrafenib) is now an approved therapy for BRAF mutation-positive metastic melanoma (reviewed in Spain L et al, Expert Opin Pharmacother 17, 1031-1038 (2016); incorporated by reference herein). A similarly attractive alternative strategy supported by emerging data is the use of molecularly-guided drug combinations for patients with AML (Chang E et al, Leukemia 30, 1025-1032 (2016); incorporated by reference herein).

Patients with acute myeloid leukemia (AML) harboring acquired chromosome aberrations in the absence of prognostically favorable t(8;21)(q22;q22), inv(16)(p13q22)/t(16;16)(p13;q22) and t(15;17)(q22;q21) form a separate category—AML with a complex karyotype (Göhring et al., Blood, 2010 Nov. 11, 116(19) pp. 3766-9 and Mrozek, K, Semin Oncol. 2008 August; 35(4): 365-377). They constitute 10-12% of all AML patents, with the incidence of complex karyotypes increasing with the more advanced age. Recent studies using molecular-cytogenetic techniques (spectral karyotyping, M-FISH) and array comparative genomic hybridization considerably improved characterization of previously unidentified, partially identified or cryptic chromosome aberrations, and allowed precise delineation of genomic imbalances. The emerging nonrandom pattern of abnormalities includes relative paucity, but not absence, of balanced rearrangements (translocations, insertions or inversions), predominance of aberrations leading to loss of chromosome material (monosomies, deletions and unbalanced translocations) that involve, in decreasing order, chromosome arms 5q, 17p, 7q, 18q, 16q, 17q, 12p, 20q, 18p and 3p, and the presence of recurrent, albeit less frequent and often hidden (in marker chromosomes and unbalanced translocations) aberrations leading to overrepresentation of segments from 8q, 11q, 21q, 22q, 1p, 9p, and 13q.

Mutations in NPM1 are usually restricted to exon 12, as shown in an analysis of 52 primary AML patients with cytoplasmic NPM1 (NPM1c), where 98% of the patients had exon 12 mutations. To date, >55 unique mutations have been identified in exon 12 of NPM1. Most mutations consist of a (net) 4 bp insertion with >95% of mutations occurring between nucleotides 960 and 961, however, there have also been cases (<5%) that occur within 10 nucleotides up or downstream. The most common mutation is called type A constituting 80% of cases; type A mutations involve duplication of TCTG (nucleotides 956-959), creating an insertion at position 960. Type B and D mutations are also fairly common, both producing 4 bp insertions at position 960. Other mutations are rare, occurring in <1% of cases. Additionally, the frequency of nonexon 12 mutations is unknown, as most large studies restrict their analysis to exon 12.

There remains great need for directed or personalized medical options for all cancers, including hematological malignancies.

### SUMMARY

Translating the genetic and epigenetic heterogeneity underlying human cancers into therapeutic strategies is an ongoing challenge. Large-scale sequencing efforts have identified that many hematologic malignancies, such as acute myeloid leukemia (AML), are driven by a spectrum of mutations and may require combinations of agents to be treated effectively. In addition, combinatorial approaches are further necessitated by the emergence of genetically heterogeneous subclones, rescue signals in the microenvironment, and tumor-intrinsic feedback pathways that all contribute to disease relapse. Ex vivo profiling of a panel of 48 drug combinations for sensitivity against 122 primary patient samples from a variety of hematologic malignancies was performed. Greater inhibition of cell viability in the presence of combinations than that observed for either single agent was used to derive a Combination Ratio as a measure of effectiveness. Combination effectiveness was referenced against diagnostic categories as well as against genetic, cytogenetic, and cellular phenotypes of specimens from the two largest disease categories of AML and chronic lymphocytic leukemia (CLL). Most strikingly, nearly all tested combinations involving a BCL-2 inhibitor showed a strong additional benefit in patients with myeloid malignancies, whereas select combinations involving PI3K, CSF1R, or bromodomain inhibitors showed preferential benefit in lymphoid malignancies. Expanded analyses of AML and CLL patients revealed specific patterns of ex vivo drug combination efficacy that are associated with select genetic, cytogenetic, and phenotypic disease subsets, warranting further evaluation. These findings highlight the heuristic value of an integrated functional genomic approach for identifying treatment strategies for hematologic malignancies.

Disclosed are methods of treating an acute myeloid leukemia or a myeloproliferative neoplasm in a subject. These methods involve administering a combination of pharmaceutical compositions including at least Arry-382 or doramapimod to the subject.

Also disclosed are methods of treating chronic lymphocytic leukemia in a subject. Such methods involve administering a combination of pharmaceutical compositions including at least quizartinib and ibrutinib to the subject or administering a combination of pharmaceutical compositions including at least JQ1 and sorafenib to the subject.

Provided is a method of treating chronic lymphocytic leukemia in a human in need thereof, the method including administering to the human a therapeutically effective amount of quizartinib, or a pharmaceutically acceptable salt thereof, and a therapeutically effective amount of ibrutinib, or a pharmaceutically acceptable salt thereof.

Also provided is a method of treating chronic lymphocytic leukemia in a human in need thereof, the method including administering to the human a therapeutically effective amount of JQ1, or a pharmaceutically acceptable salt thereof, and a therapeutically effective amount of sorafenib, or a pharmaceutically acceptable salt thereof.

Also provided is a method of treating chronic lymphocytic leukemia in a human in need thereof, the method including administering to the human a therapeutically effective amount of venetoclax, or a pharmaceutically acceptable salt thereof, and a therapeutically effective amount of trametinib, or a pharmaceutically acceptable salt thereof.

Also disclosed are methods of treating acute myeloid leukemia in a subject, where the acute myeloid leukemia is characterized by a mutation in DNMT3A. Such methods involve administering a combination of pharmaceutical compositions including at least JQ1 and palbociclib to the subject. Such methods can further involve detecting the mutation in a sample from the subject, where the sample includes peripheral blood mononuclear cells.

Provided is a method of treating acute myeloid leukemia in a human in need thereof, wherein the acute myeloid leukemia is characterized by a mutation in DNMT3A, the method including administering to the human a therapeutically effective amount of JQ1, or a pharmaceutically acceptable salt thereof, and a therapeutically effective amount of palbociclib, or a pharmaceutically acceptable salt thereof.

Also provided is a method of treating acute myeloid leukemia in a human in need thereof, the method including the steps of:
a) collecting from the patient a sample of blood;
b) detecting the presence of a mutation in DNMT3A in the sample;
c) administering to the human a therapeutically effective amount of JQ1, or a pharmaceutically acceptable salt thereof; and
d) administering to the human a therapeutically effective amount of palbociclib, or a pharmaceutically acceptable salt thereof.

Further provided is a method of diagnosing and treating acute myeloid leukemia in a human in need thereof, the method including the steps of:
a) obtaining a blood sample from the human;
b) detecting whether a mutation in DNMT3A is present in the sample;
c) diagnosing the human as having acute myeloid leukemia with the presence of a mutation in DNMT3A; and
d) administering to the human a therapeutically effective amount of JQ1, or a pharmaceutically acceptable salt thereof, and a therapeutically effective amount of palbociclib, or a pharmaceutically acceptable salt thereof.

In some embodiments of each of the methods above JQ1 is administered to the human in need thereof at individual doses of from 10 mg to 800 mg or 20 mg to 800 mg.

In some embodiments of each of the methods above palbociclib is administered to the human in need thereof at individual doses of from 1 mg to 200 mg.

As noted by Russler-Germain et al., Cancer Cell, 2014 April, 14(25(4), pp. 442-54, somatic mutations in DNMT3A, which encodes a de novo DNA methyltransferase, are found in 30% of normal karyotype acute myeloid leukemia (AML) cases. Most mutations are heterozygous and alter R882 within the catalytic domain (most commonly R882H), suggesting the possibility of dominant-negative consequences. The methyltransferase activity of R882H DNMT3A is reduced by 80% compared with the wild type (WT) enzyme. In vitro mixing of WT and R882H DNMT3A does not affect the WT activity, but coexpression of the two proteins in cells profoundly inhibits the WT enzyme by disrupting its ability to homotetramerize. AML cells with the R882H mutation have severely reduced de novo methyltransferase activity and focal hypomethylation at specific CpGs throughout AML cell genomes.

DNMT3A mutations occur in 17.1% of AML cases, occurring most often at the R882 residue of the protein, and are thought to cause loss of function (Shih et al., Nat Rev Cancer, 2012 September, 12(9), pp. 599-612; incorporated by reference herein).

More than half of DNMT3A mutations in AML samples are heterozygous missense alterations within the catalytic domain of the enzyme at residue R882, most commonly resulting in an arginine to histidine change (Ley et al., 2010; Shen et al., 2011; Thol et al., 2011; Yan et al., 2011; Marcucci et al., 2012; Ribeiro et al., 2012; each incorporated by reference herein). The high frequency of mutations at this specific site raises the possibility that this amino acid change creates a gain-of-function activity, and/or produces a protein with a dominant negative effect on the residual wild-type (WT) protein.

Within each of the methods herein concerning a mutation in DNMT3A, there is a further embodiment including each of the steps of the individual method in question in which the mutation in DNMT3A is a mutation at R882.

Within each of the methods herein concerning a mutation in DNMT3A, there is a further embodiment including each of the steps of the individual method in question in which the mutation in DNMT3A is a mutation at R882H.

Also disclosed are methods of treating acute myeloid leukemia in a subject, where the acute myeloid leukemia is characterized by a mutation NPM1. Such methods involve administering a combination of pharmaceutical compositions including at least JQ1 and sorafenib to the subject. Such methods can further involve detecting the mutation in a sample from the subject, where the sample includes peripheral blood mononuclear cells.

Provided is a method of treating acute myeloid leukemia in a human in need thereof, wherein the acute myeloid leukemia is characterized by a mutation in NPM1, the method including administering to the human a therapeutically effective amount of JQ1, or a pharmaceutically acceptable salt thereof, and a therapeutically effective amount of sorafenib, or a pharmaceutically acceptable salt thereof.

Also provided is a method of treating acute myeloid leukemia in a human in need thereof, the method including the steps of:
a) collecting from the patient a sample of blood;
b) detecting the presence of a mutation in NPM1 in the sample;
c) administering to the human a therapeutically effective amount of JQ1, or a pharmaceutically acceptable salt thereof; and
d) administering to the human a therapeutically effective amount of sorafenib, or a pharmaceutically acceptable salt thereof.

Further provided is a method of diagnosing and treating acute myeloid leukemia in a human in need thereof, the method including the steps of:
a) obtaining a blood sample from the human;
b) detecting whether a mutation in NPM1 is present in the sample;
c) diagnosing the human as having acute myeloid leukemia with the presence of a mutation in NPM1; and
d) administering to the human a therapeutically effective amount of JQ1, or a pharmaceutically acceptable salt thereof, and a therapeutically effective amount of sorafenib, or a pharmaceutically acceptable salt thereof.

In some embodiments of each of the methods above in which the human has acute myeloid leukemia with the presence of a mutation in NPM1, JQ1 is administered to the human in need thereof at individual doses of from 10 mg to 800 mg or from 20 mg to 800 mg.

In some embodiments of each of the methods above in which the human has acute myeloid leukemia with the presence of a mutation in NPM1, sorafenib is administered to the human in need thereof at individual doses of from 50 mg to 1200 mg.

Within each of the methods herein concerning a mutation in NPM1, there is a further embodiment including each of the steps of the individual method in question in which the mutation in NPM1 is a mutation at exon 12.

Within each of the methods herein concerning a mutation in NPM1, there is a further embodiment including each of the steps of the individual method in question in which the mutation in NPM1 is a mutation including an insertion of from one to four base pairs occurring within 10 nucleotides upstream or downstream of between nucleotides 960 and 961.

Within each of the methods herein concerning a mutation in NPM1, there is a further embodiment including each of the steps of the individual method in question in which the mutation in NPM1 is a mutation including a duplication of TCTG (nucleotides 956-959), creating an insertion at position 960.

Also disclosed are methods of treating acute myeloid leukemia in a subject, where the acute myeloid leukemia is characterized by a normal karyotype. Such methods involve administering a combination of pharmaceutical compositions including at least ruxolitinib and cabozantinib to the subject. Such methods can further involve assessing the karyotype of peripheral blood mononuclear cells in a sample from the subject.

Provided is a method of treating acute myeloid leukemia in a human in need thereof, wherein the acute myeloid leukemia is characterized by a normal karyotype, the method including administering to the human a therapeutically effective amount of ruxolitinib, or a pharmaceutically acceptable salt thereof, and a therapeutically effective amount of cabozantinib, or a pharmaceutically acceptable salt thereof.

Also provided is a method of treating acute myeloid leukemia in a human in need thereof, the method including the steps of:
a) collecting from the patient a sample of blood;
b) detecting the presence of a normal karyotype in the sample;
c) administering to the human a therapeutically effective amount of ruxolitinib, or a pharmaceutically acceptable salt thereof; and
d) administering to the human a therapeutically effective amount of cabozantinib, or a pharmaceutically acceptable salt thereof.

Further provided is a method of diagnosing and treating acute myeloid leukemia in a human in need thereof, the method including the steps of:
a) obtaining a blood sample from the human;
b) detecting whether a normal karyotype is present in the sample;
c) diagnosing the human as having acute myeloid leukemia with the presence of a normal karyotype; and
d) administering to the human a therapeutically effective amount of ruxolitinib, or a pharmaceutically acceptable salt thereof, and a therapeutically effective amount of cabozantinib, or a pharmaceutically acceptable salt thereof.

In some embodiments of each of the methods above in which the human has acute myeloid leukemia with the presence of a normal karyotype, ruxolitinib is administered to the human in need thereof at individual doses of from 1 mg to 200 mg or from 1 mg to 50 mg.

In some embodiments of each of the methods above in which the human has acute myeloid leukemia with the presence of a normal karyotype, ruxolitinib and cabozantinib are administered to the human in need thereof at a dose leading to an IC₅₀ CR in the human of from 1 to 200.

In some embodiments of each of the methods above in which the human has acute myeloid leukemia with the presence of a normal karyotype, cabozantinib is administered to the human in need thereof at individual doses of from 1 mg to 200 mg or from 5 mg to 120 mg.

Also disclosed are methods of treating acute myeloid leukemia in a subject, where the acute myeloid leukemia is characterized by a complex karyotype. Such methods involve administering a combination of pharmaceutical compositions including at least idelalisib and quizartinib to the subject. Such methods can further involve assessing the karyotype of peripheral blood mononuclear cells in a sample from the subject. As noted above, acute myeloid leukemia (AML) harboring ≥3 acquired chromosome aberrations in the absence of prognostically favorable t(8;21)(q22;q22), inv(16)(p13q22)/t(16;16)(p13;q22) and t(15;17)(q22;q21) form is considered AML with a complex karyotype

Provided is a method of treating acute myeloid leukemia in a human in need thereof, wherein the acute myeloid leukemia is characterized by a complex karyotype, the method including administering to the human a therapeutically effective amount of idelalisib, or a pharmaceutically acceptable salt thereof, and a therapeutically effective amount of quizartinib, or a pharmaceutically acceptable salt thereof.

Also provided is a method of treating acute myeloid leukemia in a human in need thereof, the method including the steps of:
a) collecting from the patient a sample of blood;
b) detecting the presence of a complex karyotype in the sample;
c) administering to the human a therapeutically effective amount of idelalisib, or a pharmaceutically acceptable salt thereof; and
d) administering to the human a therapeutically effective amount of quizartinib, or a pharmaceutically acceptable salt thereof.

Further provided is a method of diagnosing and treating acute myeloid leukemia in a human in need thereof, the method including the steps of:
a) obtaining a blood sample from the human;
b) detecting whether a complex karyotype is present in the sample;
c) diagnosing the human as having acute myeloid leukemia with the presence of a normal karyotype; and
d) administering to the human a therapeutically effective amount of idelalisib, or a pharmaceutically acceptable salt thereof, and a therapeutically effective amount of quizartinib, or a pharmaceutically acceptable salt thereof.

In some embodiments of each of the methods above in which the human has acute myeloid leukemia with the presence of a complex karyotype, idelalisib is administered to the human in need thereof at individual doses of from 50 mg to 1200 mg or from 50 mg to 750 mg.

In some embodiments of each of the methods above in which the human has acute myeloid leukemia with the presence of a complex karyotype, quizartinib is administered to the human in need thereof at individual doses of from 5 mg to 50 mg.

Also disclosed are methods of treating acute myeloid leukemia in a subject, where the acute myeloid leukemia is characterized by expression of CD11b. Such methods involve administering a combination of pharmaceutical compositions including at least venetoclax and JQ1 to the subject. Such methods can further involve contacting a sample including peripheral blood mononuclear cells from the subject with an antibody that binds CD11b. The antibody can include a label such as a fluorescent label that facilitates identification of CD11b positive cells by flow cytometry.

Provided is a method of treating acute myeloid leukemia in a human in need thereof, wherein the acute myeloid leukemia is characterized by expression of CD11b, the method including administering to the human a therapeutically effective amount of venetoclax, or a pharmaceutically acceptable salt thereof, and a therapeutically effective amount of JQ1, or a pharmaceutically acceptable salt thereof.

Also provided is a method of treating acute myeloid leukemia in a human in need thereof, the method including the steps of:
a) collecting from the patient a sample of blood;
b) detecting the presence of expression of CD11b in the sample;
c) administering to the human a therapeutically effective amount of venetoclax, or a pharmaceutically acceptable salt thereof; and
d) administering to the human a therapeutically effective amount of JQ1, or a pharmaceutically acceptable salt thereof.

Further provided is a method of diagnosing and treating acute myeloid leukemia in a human in need thereof, the method including the steps of:
a) obtaining a blood sample from the human;
b) detecting whether expression of CD11b is present in the sample;
c) diagnosing the human as having acute myeloid leukemia with the presence of expression of CD11b; and
d) administering to the human a therapeutically effective amount of venetoclax, or a pharmaceutically acceptable salt thereof, and a therapeutically effective amount of JQ1, or a pharmaceutically acceptable salt thereof.

Within each of the methods above wherein the acute myeloid leukemia is characterized by expression of CD11b there is a further embodiment in which the detection of CD11b expression involves contacting a sample of peripheral blood mononuclear cells from the subject with an antibody that binds CD11b and ascertaining the level of binding. In further embodiments the antibody that binds CD11b includes a label such as a fluorescent label that facilitates identification of CD11b positive cells by flow cytometry.

In some embodiments of each of the methods above in which the human has acute myeloid leukemia with the presence of expression of CD11b, venetoclax is administered to the human in need thereof at individual doses of from 5 mg to 600 mg or from 5 mg to 500 mg.

In some embodiments of each of the methods above in which the human has acute myeloid leukemia with the presence of expression of CD11b, JQ1 is administered to the human in need thereof at individual doses of from 10 mg to 800 mg or from 20 mg to 800 mg.

Also disclosed are methods of treating acute myeloid leukemia in a subject, where the acute myeloid leukemia is characterized by expression of CD58. Such methods involve administering a combination of pharmaceutical compositions including at least venetoclax and doramapimod to the subject. Such methods can further involve contacting a sample including peripheral blood mononuclear cells from the subject with an antibody that binds CD58. The antibody can include a label such as a fluorescent label that facilitates identification of CD58 positive cells by flow cytometry.

Provided is a method of treating acute myeloid leukemia in a human in need thereof, wherein the acute myeloid leukemia is characterized by expression of CD58, the method including administering to the human a therapeutically effective amount of venetoclax, or a pharmaceutically acceptable salt thereof, and a therapeutically effective amount of doramapimod, or a pharmaceutically acceptable salt thereof.

Also provided is a method of treating acute myeloid leukemia in a human in need thereof, the method including the steps of:
a) collecting from the patient a sample of blood;
b) detecting the presence of expression of CD58 in the sample;
c) administering to the human a therapeutically effective amount of venetoclax, or a pharmaceutically acceptable salt thereof; and
d) administering to the human a therapeutically effective amount of doramapimod, or a pharmaceutically acceptable salt thereof.

Further provided is a method of diagnosing and treating acute myeloid leukemia in a human in need thereof, the method including the steps of:
a) obtaining a blood sample from the human;
b) detecting whether expression of CD58 is present in the sample;
c) diagnosing the human as having acute myeloid leukemia with the presence of expression of CD58; and
d) administering to the human a therapeutically effective amount of venetoclax, or a pharmaceutically acceptable salt thereof, and a therapeutically effective amount of doramapimod, or a pharmaceutically acceptable salt thereof.

Within each of the methods above wherein the acute myeloid leukemia is characterized by expression of CD58 there is a further embodiment in which the detection of CD58 expression involves contacting a sample of peripheral blood mononuclear cells from the subject with an antibody that binds CD58 and ascertaining the level of binding. In further embodiments the antibody that binds CD58 includes a label such as a fluorescent label that facilitates identification of CD58 positive cells by flow cytometry.

In some embodiments of each of the methods above in which the human has acute myeloid leukemia with the presence of expression of CD58, venetoclax is administered to the human in need thereof at individual doses of from 5 mg to 600 mg or from 5 mg to 500 mg.

In some embodiments of each of the methods above in which the human has acute myeloid leukemia with the presence of expression of CD58, doramapimod is administered to the human in need thereof at individual doses of from 1 mg to 600 mg.

Also disclosed herein are methods of treating chronic lymphocytic leukemia in a subject, where the chronic lymphocytic leukemia is characterized by a deletion mutation in chromosome 13q. Such methods involve administering a combination of pharmaceutical compositions including at least venetoclax and palbociclib or including at least trametinib and palbociclib.

Provided is a method of treating chronic lymphocytic leukemia in a human in need thereof, wherein the chronic lymphocytic leukemia is characterized by a deletion mutation in chromosome 13q, the method including administering to the human a therapeutically effective amount of palbociclib, or a pharmaceutically acceptable salt thereof, and a therapeutically effective amount of a second agent selected from the group of venetoclax and trametinib, or a pharmaceutically acceptable salt thereof.

Provided is a method of treating chronic lymphocytic leukemia in a human in need thereof, wherein the chronic lymphocytic leukemia is characterized by a deletion mutation in chromosome 13q, the method including administering to the human a therapeutically effective amount of palbociclib, or a pharmaceutically acceptable salt thereof, and a therapeutically effective amount of venetoclax, or a pharmaceutically acceptable salt thereof.

Provided is a method of treating chronic lymphocytic leukemia in a human in need thereof, wherein the chronic lymphocytic leukemia is characterized by a deletion mutation in chromosome 13q, the method including administering to the human a therapeutically effective amount of palbociclib, or a pharmaceutically acceptable salt thereof, and a therapeutically effective amount of trametinib, or a pharmaceutically acceptable salt thereof.

Also provided is a method of treating chronic lymphocytic leukemia in a human in need thereof, the method including the steps of:
a) collecting from the patient a sample of blood;
b) detecting the presence of a deletion mutation in chromosome 13q in the sample;
c) administering to the human a therapeutically effective amount of palbociclib, or a pharmaceutically acceptable salt thereof; and
d) administering to the human a therapeutically effective amount of a second agent selected from the group of venetoclax and trametinib, or a pharmaceutically acceptable salt thereof.

Also provided is a method of treating chronic lymphocytic leukemia in a human in need thereof, the method including the steps of:
a) collecting from the patient a sample of blood;
b) detecting the presence of a deletion mutation in chromosome 13q in the sample;
c) administering to the human a therapeutically effective amount of palbociclib, or a pharmaceutically acceptable salt thereof; and
d) administering to the human a therapeutically effective amount of venetoclax, or a pharmaceutically acceptable salt thereof.

Also provided is a method of treating chronic lymphocytic leukemia in a human in need thereof, the method including the steps of:
a) collecting from the patient a sample of blood;
b) detecting the presence of a deletion mutation in chromosome 13q in the sample;
c) administering to the human a therapeutically effective amount of palbociclib, or a pharmaceutically acceptable salt thereof; and
d) administering to the human a therapeutically effective amount of trametinib, or a pharmaceutically acceptable salt thereof.

Further provided is a method of diagnosing and treating chronic lymphocytic leukemia in a human in need thereof, the method including the steps of:
a) obtaining a blood sample from the human;
b) detecting whether a deletion mutation in chromosome 13q is present in the sample;
c) diagnosing the human as having chronic lymphocytic leukemia with the presence of a deletion mutation in chromosome 13q; and
d) administering to the human a therapeutically effective amount of palbociclib, or a pharmaceutically acceptable salt thereof, and a therapeutically effective amount of a second agent selected from the group of venetoclax and trametinib, or a pharmaceutically acceptable salt thereof.
   Further provided is a method of diagnosing and treating chronic lymphocytic leukemia in a human in need thereof, the method including the steps of:
a) obtaining a blood sample from the human;
b) detecting whether a deletion mutation in chromosome 13q is present in the sample;
c) diagnosing the human as having chronic lymphocytic leukemia with the presence of a deletion mutation in chromosome 13q; and
d) administering to the human a therapeutically effective amount of palbociclib, or a pharmaceutically acceptable salt thereof, and a therapeutically effective amount of venetoclax, or a pharmaceutically acceptable salt thereof.
   Further provided is a method of diagnosing and treating chronic lymphocytic leukemia in a human in need thereof, the method including the steps of:
a) obtaining a blood sample from the human;
b) detecting whether a deletion mutation in chromosome 13q is present in the sample;
c) diagnosing the human as having chronic lymphocytic leukemia with the presence of a deletion mutation in chromosome 13q; and
d) administering to the human a therapeutically effective amount of palbociclib, or a pharmaceutically acceptable salt thereof, and a therapeutically effective amount of trametinib, or a pharmaceutically acceptable salt thereof.

In some embodiments of each of the methods above in which the human has chronic lymphocytic leukemia with the presence of a deletion mutation in chromosome 13q and palbociclib is administered with venetoclax, the palbociclib is administered to the human in need thereof at individual doses of from 1 mg to 200 mg or from 25 mg to 200 mg.

In some embodiments of each of the methods above in which the human has chronic lymphocytic leukemia with the presence of a deletion mutation in chromosome 13q and palbociclib is administered with trametinib, the palbociclib is administered to the human in need thereof at individual doses of from 1 mg to 200 mg or from 25 mg to 200 mg.

In some embodiments of each of the methods above in which the human has chronic lymphocytic leukemia with the presence of a deletion mutation in chromosome 13q, trametinib is administered to the human in need thereof at individual doses of from 0.1 mg to 5 mg.

In some embodiments of each of the methods above in which the human has chronic lymphocytic leukemia with the presence of a deletion mutation in chromosome 13q, venetoclax is administered to the human in need thereof at individual doses of from 5 mg to 600 mg or from 5 mg to 500 mg.

Also disclosed are methods of treating hematological malignancies in a subject. Such methods involve administering a combination of pharmaceutical compositions described in FIGS. 9A and 9B herein.

In any of the above methods, the pharmaceutical compositions can be administered as a co-formulation meaning that the two compositions are administered in the same formulation. Otherwise, the two pharmaceutical compositions can be administered separately with one pharmaceutical composition administered prior to the other pharmaceutical composition with a delay of seconds, minutes, hours, days, or weeks between the administrations.

In any of the above methods the combination of pharmaceutical compositions is administered at a dose equivalent to the combined IC₅₀, IC₇₀, or IC₉₀ of the combination of the pharmaceutical compositions.

### BRIEF DESCRIPTION OF THE SEVERAL VIEWS OF THE DRAWINGS

Color figures were submitted as part of the original disclosure and references to colors in the figures are provided in this description. Applicants reserve the right to present color versions of the figures disclosed herein in later proceedings.

FIGS. 1A-1E. Differential patterns of selective efficacy of small-molecule combinations relative to single agents. (1A) Unsupervised hierarchical clustering of IC₅₀ CR values for 122 leukemia patients across 48 tested combinations. IC₅₀ CR values were log-transformed and row- and column-clustered using Pearson correlation pairwise average linkage method. Darker red color (lower CR values) indicates drug combinations exhibiting higher efficacy than either single agent alone. Diagnostic category annotation of each sample is also shown. (1B) Correlation of IC₅₀ CR and AUC CR effect measure values. Shaded region indicates sample-drug pairs where the combination was more effective than either single agent (CR<1) by both effect measures. (1C) Distribution of effective drug combinations based on frequency and diagnostic category. Bar labeled “All samples” indicates diagnosis group breakdown for all 122 surveyed patient samples, for comparison. (1D) Overlap of significantly effective inhibitor combinations among four general diagnosis subgroups. Combinations where both median IC₅₀ CR and AUC CR were significantly <1 within each diagnostic subgroup are shown; combinations listed in overlapping regions were significantly effective in each subgroup. (1E) Select examples of combinations by diagnostic subgroup. Scatter plots of log-transformed IC₅₀ CR values for select combinations. Black horizontal bars represent median CR; * indicates median CR is significantly <1 in that subgroup.

FIG. 2. Clinical and genetic features of AML patients surveyed. Panels of the indicated disease-specific clinical, prognostic, mutation, cytogenetic, and surface antigen features were compared among all 58 AML patients in the study. The number of patients evaluable for each feature is given, along with (where relevant for categorical variables) the number of positive samples for a given feature. Gray boxes indicate the information was unavailable. Each patient is shown in a unique column, and samples are sorted left to right according to frequency of genetic mutations.

FIGS. 3A, 3B. Associations of selective inhibitor combination benefit with mutation, cytogenetic, and surface antigen expression features in AML. (3A) Scatter plots of combination-feature pairing test significance versus difference in median CR between subgroups. Summaries for mutation, cytogenetic, and surface antigens are shown in top, middle, and bottom panels, respectively. All plotted points correspond to combination-feature pairings where: 1) median IC₅₀ and AUC CR of the negative samples was not significantly <1, and 2) both positive and negative subgroups contained at least 15% of total evaluable samples each. Points above horizontal dashed gray line demonstrated median IC₅₀ CR and AUC CR values for positive subgroup that were significantly <1 (i.e. false discovery rate (FDR)-adjusted p<0.05). Points to right of vertical dashed gray line represent those where median IC₅₀ CR value of positive subgroup was at least 2-fold lower than that of negative subgroup. (3B) Scatter plots of log-transformed IC₅₀ CR values for select combinations by AML mutation, cytogenetic, and surface antigen expression subgroups. Black horizontal bars represent median CR value; * indicates median CR is significantly <1 in that subgroup.

FIG. 4. Clinical and genetic features of CLL patients surveyed. Panels of the indicated disease-specific clinical, mutation, cytogenetic, and surface antigen features were compared among all 42 CLL patients in the study. The number of patients evaluable for each feature is given, along with (where relevant for categorical variables) the number of positive samples for a given feature. Gray boxes indicate the information was unavailable. Each patient is shown in a unique column, and samples are sorted left to right according to frequency of cytogenetic abnormalities.

FIGS. 5A, 5B. Sensitivity of CLL patient samples harboring del(13q) to combinations with the CDK4/6 inhibitor palbociclib. (5A) Scatter plot of combination-feature pairing test significance versus difference in median CR between subgroups. All plotted points correspond to combination-feature pairings where: 1) the median IC₅₀ and AUC CR of the negative samples was not significantly <1, and 2) both the positive and negative subgroups contained at least 15% of the total evaluable samples each. Points above the horizontal dashed gray line demonstrated median IC₅₀ CR and AUC CR values for the positive subgroup that were significantly <1 (i.e. FDR-adjusted p<0.05). Points to the right of the vertical dashed gray line represent those where the median IC₅₀ CR value of the positive subgroup was at least 2-fold lower than that of the negative subgroup. (5B) Scatter plot of log-transformed IC₅₀ CR values for select combinations effective in del(13q)-positive CLL samples. Black horizontal bars represent the median CR value; * indicates the median CR is significantly <1 in that subgroup.

FIG. 6 is a summary matrix of combinations, targeted pathways, and malignancy-selective efficacy. For each inhibitor combination shown, shaded boxes indicate the primary signaling pathways targeted. Orange, green, and purple shading represent combinations where both the median IC₅₀ CR and AUC CR values were significantly less than 1 among myeloid patient samples (AML or MDS/MPN), lymphoid patient samples (CLL or ALL), or both, respectively. Targets of combinations for which the median CR values were not significantly effective among any of the diagnostic subgroups are shaded in gray.

FIG. 7. Single-agent IC₅₀ heatmap for all 122 evaluated leukemia patient samples. IC₅₀ values for 122 leukemia patients across each of the 21 individually tested small-molecule inhibitors were normalized to the maximum tested concentration (1 μM for dasatinib, 10 μM for all other inhibitors) and log-transformed. Patient samples are represented in columns in the identical clustered order as identified from the corresponding combination data in FIG. 1A. Darker red color indicates increased sensitivity, and the diagnostic category annotation of each sample is shown.

FIGS. 8A, 8B. Clustering of AUC-based effect measures of combination and single-agent efficacy for all 122 evaluated leukemia patient samples. (8A) Unsupervised hierarchical clustering of AUC CR values for 122 leukemia patients across 48 tested combinations. AUC CR values (defined as the ratio of the combination AUC to that of the smallest AUC of either drug alone) were log-transformed and row- and column-clustered using a Pearson correlation pairwise average linkage method. Darker red color indicates lower CR values, and diagnostic category annotation of each sample is shown. (8B) Single-agent AUC heatmap for all 122 evaluated leukemia patient samples. AUC values for all samples across each of the 21 individually tested small-molecule inhibitors were normalized to the maximum possible AUC value of 286.27 and log-transformed. Patient samples are represented in columns in the identical clustered order as identified from the corresponding combination data in 8A. Darker red color indicates increased sensitivity, and the diagnostic category annotation of each sample is shown.

FIGS. 9A-9C. Distribution of IC₅₀ and AUC combination ratios across all tested patient samples. For each combination tested, frequency of samples with CR values are shown for (9A) IC₅₀ and (9B) AUC effect measures. Two different sets of cutpoints were examined: CR>1/CR=1/CR<1 or CR>2/CR 0.5-2/CR<0.5. Combinations are sorted according to frequency of samples with CR values <1 or <0.5, respectively. (C) Representative dose-response curves for AML or CLL patients exhibiting CR values <1 for select combinations shown in FIG. 1E. Probit-derived curves for each single-agent and the combination are shown.

FIGS. 10A-10C. Validation of combination selectivity between AML and CLL. (10A) The difference of median IC₅₀ CR values (AML-CLL) was computed for each of 48 indicated combinations using the Hodges-Lehmann method. The median difference is represented by a closed circle, and the 95% confidence interval is shown as the colored bar. AML-selective and CLL-selective combinations are colored orange or green, respectively. (10B) Spearman correlation of log-transformed median IC₅₀ CR and AUC CR values between the discovery sample cohort (n=122) and independent validation cohort (n=151). (10C) Validation of select effective combinations within AML or CLL diagnostic subgroups. Scatter plots of log-transformed IC₅₀ CR values for the indicated combinations. Black horizontal bars represent median CR; FDR-adjusted p-values (Wilcoxon Sign Rank test of median) are shown.

FIG. 11. Apoptosis induction for venetoclax combinations in AML cell lines. Three human AML cell lines (MOLM13, HL-60, and OCI-AML2) were cultured in the presence of venetoclax alone or in combination with doramapimod, ruxolitinib, idelalisib, or trametinib. All drug concentrations used were 50 nM. After 48 h, the percentage of cells positive for annexin V staining were measured by Guava Nexin assay (Millipore). Bars indicate the mean of three replicates ±S.D.

FIG. 12. Summary of patient sample cohort by diagnosis category.

FIG. 13. Selective inhibitor combination sensitivities by feature among AML and CLL patient samples surveyed.

FIG. 14. Exemplary sequences supporting the disclosure including SEQ ID NOs. 1-8.

### DETAILED DESCRIPTION

In the absence of a portfolio of therapeutics that target specific mutations, ex vivo functional screening was used to identify drug sensitivities in primary samples from patients with various hematologic malignancies. Based on data accumulated from this assay to date, many instances of ex vivo sensitivity to small-molecule kinase inhibitors were validated against known genetic targets (e.g. BCR-ABL, FLT3-ITD, RAS, etc.) (Tyner J W et al, Cancer Res 73, 285-296 (2013); incorporated by reference herein). This observation suggests that a similar screening platform may identify combinations of targeted agents that are more effective than either of their respective single agents, thus defining and enabling a rational program for selecting clinically-relevant combinatorial therapies. Thus, to identify new therapeutic combinations for AML and other hematologic malignancies, the sensitivity of primary patient samples to various drug combinations was assessed using this ex vivo platform.

### Terms

Acute myeloid leukemia (AML): a rapidly progressing cancer of the blood and bone marrow that affects a group of white blood cells called the myeloid cells. AML can also be referred to as acute myelogenous leukemia, acute myeloblastic leukemia, acute granulocytic leukemia and acute nonlymphocytic leukemia.

Acute lymphoblastic leukemia (ALL): a rapidly progressing cancer of the blood and bone marrow characterized by the creation of immature rather than mature blood cells. ALL primarily affects lymphocytes and is also known as acute lymphocytic leukemia.

Binding: an association between two substances or molecules such as the association of an antibody with a cell surface marker. As described herein, stable binding (or detectable binding) means that a macromolecule such as an antibody can bind to another macromolecule such as a polypeptide in a manner that can be detected. Binding can be detected by any procedure known to one skilled in the art, such as by physical or functional properties. Binding can also be detected by visualization of a label (such as a fluorescent label) conjugated to one of the molecules. Specific binding means that a macromolecule such as an antibody binds to members of a class of macromolecules to the exclusion of macromolecules not in that class (binding to non-specific antibody binding macromolecules such as protein A, Fc receptors, etc. is excepted).

Biomarker (marker): Molecular, biological or physical attributes that characterize a physiological, cellular, or disease state and that can be objectively measured to detect or define disease progression or predict or quantify therapeutic responses. A biomarker is a characteristic that is objectively measured and evaluated as an indicator of normal biologic processes, pathogenic processes, or pharmacologic responses to a therapeutic intervention. A biomarker may be any molecular structure produced by a cell or organism. A biomarker may be expressed inside any cell or tissue; accessible on the surface of a tissue or cell; structurally inherent to a cell or tissue such as a structural component, secreted by a cell or tissue, produced by the breakdown of a cell or tissue through processes such as necrosis, apoptosis or the like; or any combination of these. A biomarker may be any protein, carbohydrate, fat, nucleic acid, catalytic site, or any combination of these such as an enzyme, glycoprotein, cell membrane, virus, cell, organ, organelle, or any uni- or multimolecular structure or any other such structure now known or yet to be disclosed whether alone or in combination. A biomarker can also be a discrete cellular entity such as a circulating leukemia cell expressing particular cell surface markers including CD11b or CD58.

Chronic lymphocytic leukemia (CLL): a slower progressing cancer of the blood and bone marrow that affects lymphocytes.

Contacting: Placing within an environment where direct physical association occurs, including contacting of a solid with a solid, a liquid with a liquid, a liquid with a solid, or either a liquid or a solid with a cell or tissue, whether in vitro or in vivo. Contacting can occur in vitro with isolated cells or tissue or in vivo by administering to a subject.

Effective Amount: An amount of an agent that is sufficient to generate a desired response such as reducing or eliminating a sign or symptom of a condition or a disease. An effective amount also encompasses an effective amount of a first agent and an effective amount of a second agent administered in combination with the first agent. In some examples, the effective amount of the two combined agents is less than that of either agent when administered alone.

Hematological malignancy: a general term for cancers that affects the blood or bone marrow.

Label: A label can be any substance capable of aiding a machine, detector, sensor, device, column, or enhanced or unenhanced human eye from differentiating a labeled composition from an unlabeled composition. Labels may be used for any of a number of purposes and one skilled in the art will understand how to match the proper label with the proper purpose. Examples of uses of labels include purification of biomolecules, identification of biomolecules, detection of the presence of biomolecules, detection of protein folding, and localization of biomolecules within a cell, tissue, or organism. Examples of labels include: radioactive isotopes or chelates thereof; dyes (fluorescent or nonfluorescent), stains, enzymes, nonradioactive metals, magnets, protein tags, fluorescent proteins, any antibody epitope, any specific example of any of these; any combination between any of these, or any label now known or yet to be disclosed. A label may be covalently attached to a biomolecule or bound through hydrogen bonding, Van Der Waals or other forces. A label may be covalently or otherwise bound to the N-terminus, the C-terminus or any amino acid of a polypeptide or the 5′ end, the 3′ end or any nucleic acid residue in the case of a polynucleotide.

Myeloproliferative neoplasms or myelodysplastic syndromes (MPN or MDS/MPN): blood cancers that are characterized by the overproduction of white blood cells, red blood cell, or platelets. Examples of MPNs include polycythemia vera, essential thrombocythemia, and myelofibrosis.

Subject: A living multicellular vertebrate organism, a category that includes, for example, mammals and birds. A “mammal” includes both human and non-human mammals, such as mice. In some examples, a subject is a patient, such as a patient diagnosed with cancer. In other examples, a subject is a patient yet to be diagnosed with cancer.

Treatment: any therapeutic intervention that ameliorates a sign or symptom of a disease or pathological condition. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of metastases, an improvement in the overall health or well-being of the subject, or by other clinical or physiological parameters associated with a particular disease. A “prophylactic” treatment is a treatment administered to a subject who does not exhibit signs of a disease or exhibits only early signs for the purpose of decreasing the risk of developing pathology. A “therapeutic” treatment is a treatment administered after the development of significant signs or symptoms of the disease.

Methods to detect genetic alterations. In particular embodiments, chromosomal aberrations or chromosome 13q deletions can be assessed using GISTIC2.0 scores and R software with the copy number package. Chromosomal aberrations or chromosome 13q deletions can also be detected by comparing allele intensities to referenced baselines using, for example, the Partek Genomics Suite. Genetic alterations can also be detected by comparative genomic hybridization (CGH). CGH is useful for identifying copy number variation, chromosomal translocation, and/or large chromosomal insertions or deletions (e.g., 40 kb or more). For array-based CGH, target sequences (e.g., genomic fragments from a reference genome) can be present on a tile array, and hybridized to sample DNA that has been labelled with a fluorophore (control DNA can be labelled with a different fluorophore). Imaging techniques can be used to detect fluorescent signals, and copy number can be relatively compared between sample DNA and control DNA. Exemplary chromosomal staining materials include Giemsa stain and Orcein stain.

In particular embodiments, genetic alterations can be detected by multiplex ligation-dependent probe amplification (MLPA). MLPA is a PCR technique that can be used to simultaneously detect alterations in copy number (such as chromosome loss or gain, or gene amplification or deletion), point mutations, and/or indels. The technique uses a forward and reverse primer that recognize adjacent target sites on DNA. When both primers bind to the target site, they can become ligated to form a probe. Next, the ligated probes can be amplified by PCR. If each probe that detects different target sites is designed to be a unique size, the amplified probes can be resolved by size and quantified by label detection (e.g. fluorescent tag). The quantified probes can be compared to a reference, such as a control sample with a known target copy number.

In particular embodiments, genetic alterations can be detected by fluorescent in situ hybridization (FISH). Probes that bind to target nucleic acids can be linked to a primary label (e.g., biotin). Next, the probes can be hybridized to the target DNA/RNA in a sample (e.g., a tissue section or cell monolayer), and binding of the probe to the target can be detected by measuring a signal from a secondary label (e.g., a fluorescently-tagged antibody specific for the primary label). In particular embodiments, the FISH probe is a bacterial artificial chromosome (BAC) probe, which can be labeled, for example, with a fluorescent tag. In particular embodiments, chromosome 13q deletions can be assessed using FISH. Techniques to detect chromosome 13q deletions by FISH are described, for example, in Rouault, A., et al., PLOS One, 2012. https://doi.org/10.1371/journal.pone.0052079 which is incorporated by reference herein. Chromosome 13q deletions can be in the genomic region hg19 chr13: 44921196-45086777.

In particular embodiments, genetic alterations can be detected by gene sequencing. Examples of genetic alterations that can be detected by gene sequencing include point mutations, indels, and chromosomal translocation. Gene sequencing techniques that generate long reads can be useful for detecting gene amplifications and genetic alterations in repeat-rich regions of the genome. Examples of sequencing techniques that can be used for detecting genetic alterations include Sanger sequencing and next generation sequencing, such as pyrosequencing or IIlumina® (IIlumina, Inc., San Diego, Calif.) sequencing by synthesis. Gene sequencing techniques can be useful for detecting heterogenous genetic alterations (e.g., in DNMT3A or NPM1). Heterogenous genetic alterations can refer to variation in the genetic alterations (e.g., SNPs, indels, large insertions/deletions, and/or chromosomal deletions/translocations) that are associated with a health condition and/or disease outcome. For example, heterogenous genetic alterations within exon 12 of NPM1 are associated with AML. In particular embodiments, genetic alterations in exon 12 of NPM1 can detected by sequencing the amplified exon. Examples of PCR primers that can be used for amplifying exon 12 of NPM1 prior to sequencing can be found in Szankasi, P., et al., J Mol Diagn. 2008 May; 10(3): 236-241 which is incorporated by reference herein. An exemplary set of primers that can be used to amplify NPM1 prior to sequencing is GATGTCTATGAAGTGTTGTGGTTCC (forward primer, SEQ ID NO: 9), and GGACAGCCAGATATCAACTG (reverse primer, SEQ ID NO: 10).

In particular embodiments, genetic alterations can be detected by PCR amplification. Genetic alterations such as point mutations and/or short indels (e.g. 1-5 nucleotides) can be detected by allele specific PCR. Genetic alterations such as large deletions, insertions, and/or translocation can be detected by PCR by designing primers that bind to the break points of the indel or translocation. In particular embodiments genetic alterations that change an RNA sequence or the expression level of an RNA can be detected by rt-PCR.

In particular embodiments, genetic alterations can be detected by allele specific PCR. Allele specific PCR refers to a PCR assay that can discriminate between nucleotide differences at a single position. For example, allele specific PCR can be performed by using (i) a single reverse primer and (ii) two distinct allele-specific forward primers with different tails that can amplify allele-specific amplicons of different lengths, and the amplicons of different lengths can be resolved by agarose gel electrophoresis. As another example of allele specific PCR, two PCR reactions can be performed with the same sample, each with a distinct allele-specific forward primer, and the allele present in the sample can be determine based on the forward primer that yields a PCR product. The starting material for allele specific PCR can be, for example, genomic DNA or RNA transcribed from a gene of interest. Primer design for allele specific PCR is described, for example, in Liu, J. et al., Plant Methods. 2012; 8: 34 which is incorporated by reference herein.

In particular embodiments, allele specific PCR is used to detect gene mutations in the DNMT3A gene (e.g., related to DNMT3A R882 protein variants) and/or mutations in the NPM1 gene (particularly in exon 12 and/or around nucleotide position 960). In particular embodiments, allele specific PCR primers are designed to detect DNMT3A R882 variants. Examples of DNMT3A 882 variants include R882H (gene variant 2645G>A), R882C (gene variant 2644C>T), R882S (2644C>A), R882P (gene variant 2645G>C), and R882G (gene variant 2644C>G). Allele specific PCR to detect a DNMT3A R882 variant is described for example, in Berenstein, R., et al., J Exp Clin Cancer Res. 2015; 34(1): 55; and Ploen, G., et al., Br J Haematol. 2014 August; 167(4): 478-486 both of which are incorporated by reference herein. Examples of allele-specific PCR primers that can be used to detect DNMT3A R882 variants include the forward primers: GACGTCTCCAACATGACCCG (DMNT3A:R882 wt, SEQ ID NO: 11); TACTGACGTCTCCAACATGACCT (DMNT3A:R882C, SEQ ID NO: 12); ACGTCTCCAACATGAGCCAAT (DMNT3A:R882H, SEQ ID NO: 13); TACTGACGTCTCCAACATGAACA (DMNT3A:R882S, SEQ ID NO: 14); ACGTCTCCAACATGAGCCCAT (DNMT3A:R882P, SEQ ID NO: 15), and TACTGACGTCTCCAACATGAACG (DMNT3A:R882G, SEQ ID NO: 16); and the reverse primer GTGTCGCTACCTCAGTTTGCC (SEQ ID NO: 17).

SEQ ID NOs. 1-17 are provided for reference and convenience. Those of ordinary skill in the art can access additional supporting sequences from publicly available databases. Further, respective binding primers and probes can be generated based on these sequences and publicly available programs.

In particular embodiments, genetic alterations can be detected by immunostaining techniques. Genetic alterations that change the sequence of a protein or alter the expression of a protein can be detected using binding domains (e.g., antibodies) that specifically bind to a protein (e.g., a mutant protein). Examples of immunostaining techniques that can be used to detect proteins include enzyme linked immunosorbent assay, flow cytometry, Western blotting, and immunohistochemistry.

In particular embodiments, the immunostaining technique can utilize an antibody that detects an epitope of a cell surface protein, such as CD11b or CD58.

In particular embodiments, an antibody that binds to an epitope of CD11b can be used. Examples of commercially available antibodies that bind an epitope of CD11b include clone M1/70 (available from BIOLEGEND™) and the NOVUS antibody #NB110-89474. Methods of detecting cell surface CD11b are described, for example, in Zheng, C., et al., Proc Natl Acad Sci USA. 2015 Dec. 29; 112(52): E7239-E7248 which is incorporated by reference herein.

In particular embodiments, an antibody that binds to an epitope of CD58 can be used. Examples of commercially available antibodies that bind an epitope of CD58 include AF1689 (available from R&D SYSTEMS™) clone T52/9 (available from BIOLEGEND™), and clone MEM-63 (available from THERMOFISHER™). Methods of detecting cell surface CD58 are described, for example, in Challa-Malladi, M., et al., Cancer Cell. 2011 Dec. 13; 20(6): 728-740 which is incorporated by reference herein. “Is expressed” in relation to CD11b and CD58 should be interpreted in keeping with the experimental results and figures described herein (e.g., positive).

### Compounds.

JQ1 is also known as JQ 1, JQ 1(+), and (6S)-4-(4-Chlorophenyl)-2,3,9-trimethyl-6H-thieno[3,2-f][1,2,4]triazolo[4,3-a][1,4]diazepine-6-acetic acid 1,1-dimethylethyl ester, having the structure:

Ibrutinib has the structure:

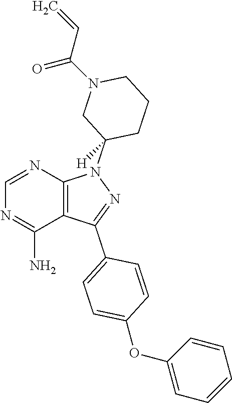 [SMILES: C=CC(=O)N1CCC[C@@H](n2nc(-c3ccc(Oc4ccccc4)cc3)c3c(N)ncnc32)C1]

Particular embodiments include CDK4/6 inhibitors. Exemplary CDK4/6 inhibitors include PD-0332991, Flavopiridol, AT7519M, P276-00, SCH 727965, AG-024322, LEE011, LY2835219, P1446A-05, BAY 1000394, SNS-032, pyrido[2,3-d]pyrimidines (e.g., pyrido[2,3-d]pyrimidin-7-ones and 2-amino-6-cyano-pyrido[2,3-d]pyrimidin-4-ones), triaminopyrimidines, aryl[a]pyrrolo[3,4-d]carbazoles, nitrogen-containing heteroaryl-substituted ureas, 5-pyrimidinyl-2-aminothiazoles, benzothiadiazines, acridinethiones, and isoquinolones (see e.g., (see, e.g., US 20140080838 which is incorporated by reference herein). CDK4/6 inhibitors are also described in, for example, US 20140031325; US 20130303543; US 2007/0027147; US 2003/0229026; US 2004/0048915; US 2004/0006074; and US 2007/0179118 each individually incorporated by reference herein. In particular embodiments, the selected CDK4/6 inhibitor includes palbociclib.

Palbociclib is chemically described as 6-acetyl-8-cyclopentyl-5-methyl-2-[[5-(1-piperazinyl)-2-pyridinyl]amino]pyrido[2,3-d]pyrimidin-7(8H)-one. The structure of palbociclib includes:

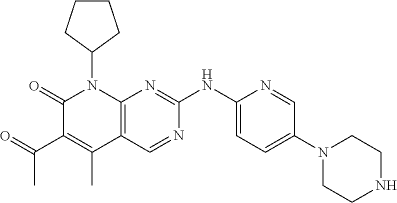 [SMILES: CC(=O)c1c(C)c2cnc(Nc3ccc(N4CCNCC4)cn3)nc2n(C2CCCC2)c1=O]

US 20170240543 provides crystalline forms of palbociclib; U.S. Pat. No. 6,936,612 provides a process for the preparation of palbociclib hydrochloride; U.S. Pat. No. 7,781,583 provides a process for the preparation of palbociclib isethionate; U.S. Pat. No. 7,863,278 provides polymorphs of various salts of palbociclib; WO 2014/128588 provides crystalline Forms A and B of palbociclib. Each is incorporated by reference herein for its relevant teachings.

Examples of a tyrosine kinase inhibitors include tyrphostin 23, tyrphostin 25, tyrphostin 46, tyrphostin 47, tyrphostin 51, tyrphostin AG 126; tyrphostin AG 825; tyrphostin Ag 1288; tyrphostin Ag 1295; geldanamycin; genistein; PP1 (also known as 1H-pyrazolo[3,4-d]pyrimidin-4-amine, 1-(1,1-dimethylethyl)-3-(1-naphthalenyl)-(9Cl)); PP2 (also known as 1H-Pyrazolo[3,4-d]pyrimidin-4-amine, 3-(4-chlorophenyl)-1-(1,1-dimethylethyl)-(9Cl));. Piceatannol (also known as 1,2-benzenediol, 4-[(1E)-2-(3,5-dihydroxyphenyl)ethenyl]-(9Cl)); Tyrphostin AG 490; 2-naphthyl vinyl ketone; 2-propenamide, 2-cyano-3-(3,4-dihydroxyphenyl)-N-phenyl-(2E)-(9Cl); tyrphostin Ag 1478; lavendustin A; and 3-pyridineacetonitrile, α-[(3,5-dichlorophenyl)methylene]-, (αZ)-(9Cl). In particular embodiments, the selected tyrosine kinase inhibitor is sorafenib. The structural formula for sorafenib includes:

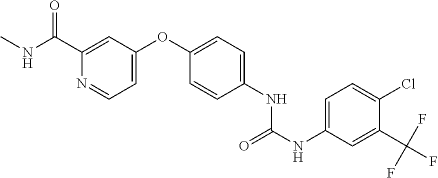 [SMILES: CNC(=O)c1cc(Oc2ccc(NC(=O)Nc3ccc(Cl)c(C(F)(F)F)c3)cc2)ccn1]

Sorafenib tosylate (also known as BAY 43-9006), is the tosylate salt of sorafenib. Sorafenib tosylate has the chemical name 4-(4-{3-[4-Chloro-3(trifluoromethyl)phenyl]ureido}phenoxy)-N 2-methylpyridine-2-carboxamide 4methylbenzenesulfonate and its structural formula includes:

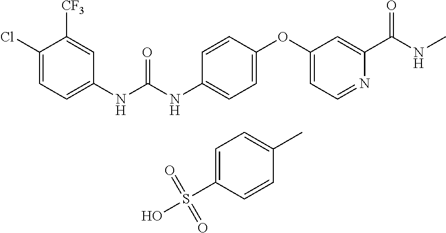

In particular embodiments, the selected tyrosine kinase inhibitor is ruxolitinib (INC424). The structural formula for ruxolitinib includes:

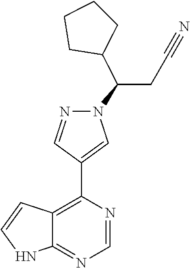 [SMILES: N#CC[C@H](C1CCCC1)n1cc(-c2ncnc3[nH]ccc23)cn1]

Examples of phosphoinositide-3 kinase (PI3) inhibitors include wortmannin, LY294002, XL-147, CAL-120, ONC-21, AEZS-127, ETP-45658, PX-866, GDC-0941, BGT226, BEZ235, and XL765. In particular embodiments, the selected PI3 inhibitor is idelalisib. Idelasib's structural formulation includes:

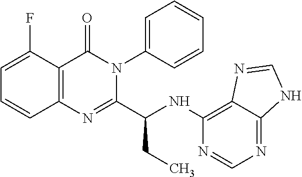 [SMILES: CC[C@H](Nc1ncnc2[nH]cnc12)c1nc2cccc(F)c2c(=O)n1-c1ccccc1]

Examples of FLT3 inhibitors include cabozantinib, ponatinib, Midostaurin, Pacritinib, quizartinib, gilteritinib, AKN-028, AT-9283, Crenolanib, ENMD-2076, Famitinib, Dovitinib, PLX-3397. Examples of CSF1R inhibitors include GW2580 [5-(3-Methoxy-4-((4-methoxybenzyl)oxy)benzyl)pyrimidine-2,4-diamine]; KI20227 {N-{4-[(6,7-dimethoxy-4-quinolyl)oxy]-2-methoxyphenyl}-NO-[1-(1,3-thiazole-2-yl)ethyl]urea}; HY-13075 {4-cyano-N-[4-(4-methylpiperazin-1-yl)-2-(4-methylpiperidin-1-yl)phenyl]-1H-pyrrole-2-carboxamide}, cFMS Receptor Inhibitor II {4-(3,4-Dimethylanilino)-7-(4-pyridyl)quinoline-3-carboxamide}, cFMS Receptor Inhibitor III {4-(3,4-Dimethylanilino)-7-(4-(methylsulfonyl)phenyl)quinoline-3-carboxamide}, cFMS Receptor Inhibitor IV {5-Cyano-N-(2,5-di(piperidin-1-yl)phenyl)furan-2-carboxamide, CSF-1 Receptor Inhibitor IV} and ARRY-382. In particular embodiments, the selected FLT3/CSF1R inhibitor is ARRY-382. In particular embodiments, the selected FLT3/CSF1R inhibitor is quizartinib. Quizartinib's structural formula includes:

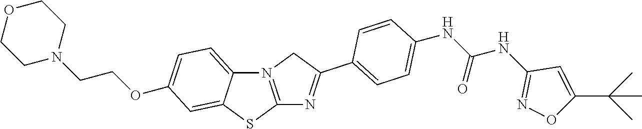

Examples of Bcl2 inhibitors include ABT-737, Obatoclax mesylate, Navitoclax, or TW-37 (see US 20140080838 incorporated by reference herein). In particular embodiments, the selected Bcl2 inhibitor is venetoclax. Venetoclax's structural formula includes:

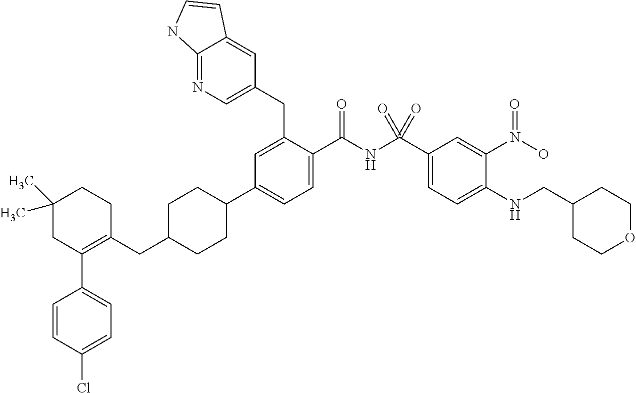

Exemplary p38MAPK inhibitors belong to the classes of 4,5-diaryl-imizadoles, 2,4,5-triarylimidazoles and 3,4,5-triarylimidazoles, such as pyridinylimidazoles including pyridinylimidazoles containing pyridinyl-4-yl and 4-fluorophenyl groups. p38MAPK inhibitors and methods of synthesizing are described in, for example, Wagner et al. 2000 (Arch. Pharm. Pharm. Med. Chem. 333 Suppl. 1, 2000: 97); U.S. Pat. Nos. 4,585,771; 4,461,298; 4,528,298; 4,402,960; 4,461,770; 4,608,382; 4,584,310; WO 00/17192; WO 95/00501; WO 99/03837; WO 93/14081; and WO 88/01167. In particular embodiments, the selected p38MAPK inhibitor is doramapimod. Doramapimod's structural formula includes:

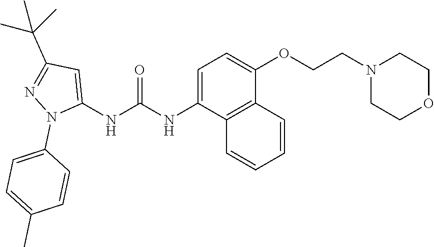 [SMILES: Cc1ccc(-n2nc(C(C)(C)C)cc2NC(=O)Nc2ccc(OCCN3CCOCC3)c3ccccc23)cc1]

Examples of MEK inhibitors include Nexavar® (sorafenib tosylate), butanedinitrile, and bis[amino[2-aminophenyl)thio]methylene]-(9Cl). In particular embodiments, the selected MEK inhibitor is trametinib. Trametinib's structural formula includes:

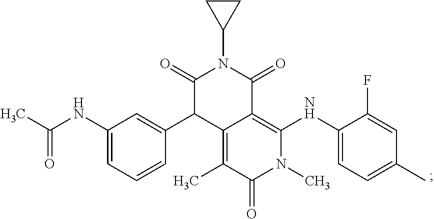 [SMILES: CC(=O)Nc1cccc(C2C(=O)N(C3CC3)C(=O)c3c2c(C)c(=O)n(C)c3Nc2ccc(C)cc2F)c1]

As indicated, salts of these compounds may also be used. In particular embodiments, salts of the compounds include those prepared with an organic acid or an inorganic acid. Examples of organic acids include acetic acid, trifluoroacetic acid, trichloroacetic acid, propionic acid, glycolic acid, pyruvic acid, succinic acid, benzoic acid, cinnamic acid, mandelic acid, methanesulphonic acid, para-toluenesulphonic acid, salicylic acid, picric acid, citric acid, oxalic acid, tartaric acid, malonic acid, maleic acid, camphor-sulphonic acid and fumaric acid. Examples of inorganic acids include hydrohalic acids such as hydrochloric acid and hydrobromic acid, sulphuric acid, nitric acid and phosphoric acid.

In particular embodiments, salts of the compounds also include those prepared with an organic base or an inorganic base. Examples of organic bases include amines such as aliphatic or aromatic primary, secondary or tertiary amines such as methylamine, ethylamine, ethanolamine, propylamine, isopropylamine, the four isomers of butylamine, dimethylamine, diethylamine, diethanolamine, dipropylamine, diisopropylamine, di-n-butylamine, pyrrolidine, piperidine, morpholine, diethanolphenylamine, trimethylamine, triethylamine, tripropylamine, quinuclidine, pyridine, quinoline and isoquinoline. Examples of inorganic bases include hydroxides of alkali metals or of alkaline-earth metals or carbonates of alkali metals or of alkaline-earth metals. Specific examples of these bases include potassium hydroxide, sodium hydroxide, lithium hydroxide, calcium hydroxide, potassium carbonate, sodium carbonate and calcium carbonate.

### Pharmaceutical Compositions.

The compounds disclosed herein may be included in pharmaceutical compositions (including therapeutic and prophylactic formulations), typically combined together with one or more pharmaceutically acceptable carriers (known equivalently as vehicles) and, optionally, other therapeutic ingredients.

Such pharmaceutical compositions can formulated for administration to subjects by a variety of mucosal administration modes, including by oral, rectal, intranasal, intrapulmonary, intravitrial, or transdermal delivery, or by topical delivery to other surfaces including the eye. Optionally, the compositions can be administered by non-mucosal routes, including by intramuscular, subcutaneous, intravenous, intra-arterial, intra-articular, intraperitoneal, intrathecal, intracerebroventricular, or parenteral routes. In other examples, the compound can be administered ex vivo by direct exposure to cells, tissues or organs originating from a subject.

To formulate the pharmaceutical compositions, the compound can be combined with various pharmaceutically acceptable additives. Desired additives include pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween®-80), solubility enhancing agents (for example, cyclodextrins and derivatives thereof), stabilizers (for example, serum albumin), and reducing agents (for example, glutathione) can be included.

When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of 0.3 to 3.0, such as 0.5 to 2.0, or 0.8 to 1.7. The compound can be dispersed in any pharmaceutically acceptable carrier, which can include a hydrophilic compound having a capacity to disperse the compound, and any desired additives. The carrier can be selected from a wide range of suitable compounds, including copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a carrier, for example, polylactic acid, poly(lactic acid-glycolic acid) copolymer, polyhydroxybutyric acid, poly(hydroxybutyric acidglycolic acid) copolymer and mixtures thereof.

Alternatively or additionally, synthetic fatty acid esters such as polyglycerin fatty acid esters, sucrose fatty acid esters and the like can be employed as carriers. Hydrophilic polymers and other vehicles can be used alone or in combination, and enhanced structural integrity can be imparted to the vehicle by partial crystallization, ionic bonding, cross-linking and the like. The carrier can be provided in a variety of forms, including fluid or viscous solutions, gels, pastes, powders, microspheres, and films for direct application to a mucosal surface.

The compound can be combined with the carrier according to a variety of methods, and release of the compound can be by diffusion, disintegration of the vehicle, or associated formation of water channels. In some circumstances, the compound is dispersed in microcapsules (microspheres) or nanoparticles prepared from a suitable polymer, for example, 5-isobutyl 2-cyanoacrylate (see, for example, Michael et al., J. Pharmacy Pharmacol. 43, 1-5, (1991)), and dispersed in a biocompatible dispersing medium, which yields sustained delivery and biological activity over a protracted time.

Pharmaceutical compositions for administering the compound can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

In certain embodiments, the compound can be administered in a time release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the compound and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present disclosure include polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acidco-glycolic acid), poly(D-lactic acid-co-glycolic acid), and poly(L-lactic acid-coglycolic acid). Other useful biodegradable or bioerodible polymers include such polymers as poly(epsilon-caprolactone), poly(epsilon-aprolactone-CO-lactic acid), poly(epsilon.-aprolactone-CO-glycolic acid), poly(betahydroxy butyric acid), poly(alkyl-2-cyanoacrilate), hydrogels, such as poly(hydroxyethyl methacrylate), polyamides, poly(amino acids) (for example, L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

The pharmaceutical compositions of the disclosure typically are sterile and stable under conditions of manufacture, storage and use. Sterile solutions can be prepared by incorporating the compound in the required amount in an appropriate solvent with one or a combination of ingredients enumerated herein, as required, followed by filtered sterilization. Generally, dispersions are prepared by incorporating the compound and/or other biologically active agent into a sterile vehicle that contains a basic dispersion medium and the required other ingredients from those enumerated herein. In the case of sterile powders, methods of preparation include vacuum drying and freeze-drying which yields a powder of the compound plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

In particular embodiments, JQ1 and palbociclib; JQ1 and sorafenib; cabozantinib and ruxolitinib; idelalisib and quizartinib; ibrutinib and quizartinib; venetoclax and KU; venetoclax and doramapimod; venetoclax and trametinib; palbociclib and venetoclax; and/or palbociclib and trametinib are co-formulated.

Also provided is the use of a therapeutically effective amount of JQ1, or a pharmaceutically acceptable salt thereof, and a therapeutically effective amount of palbociclib, or a pharmaceutically acceptable salt thereof, in the preparation of a medicament for the treatment of acute myeloid leukemia.

Also provided is the use of a therapeutically effective amount of JQ1, or a pharmaceutically acceptable salt thereof, and a therapeutically effective amount of sorafenib, or a pharmaceutically acceptable salt thereof, in the preparation of a medicament for the treatment of acute myeloid leukemia.

Also provided is the use of a therapeutically effective amount of cabozantinib, or a pharmaceutically acceptable salt thereof, and a therapeutically effective amount of ruxolitinib, or a pharmaceutically acceptable salt thereof, in the preparation of a medicament for the treatment of acute myeloid leukemia.

Also provided is the use of a therapeutically effective amount of idelalisib, or a pharmaceutically acceptable salt thereof, and a therapeutically effective amount of quizartinib, or a pharmaceutically acceptable salt thereof, in the preparation of a medicament for the treatment of acute myeloid leukemia.

Also provided is the use of a therapeutically effective amount of ibrutinib, or a pharmaceutically acceptable salt thereof, and a therapeutically effective amount of quizartinib, or a pharmaceutically acceptable salt thereof, in the preparation of a medicament for the treatment of acute myeloid leukemia.

Also provided is the use of a therapeutically effective amount of venetoclax, or a pharmaceutically acceptable salt thereof, and a therapeutically effective amount of JQ1, or a pharmaceutically acceptable salt thereof, in the preparation of a medicament for the treatment of acute myeloid leukemia.

Also provided is the use of a therapeutically effective amount of venetoclax, or a pharmaceutically acceptable salt thereof, and a therapeutically effective amount of doramapimod, or a pharmaceutically acceptable salt thereof, in the preparation of a medicament for the treatment of acute myeloid leukemia.

Also provided is the use of a therapeutically effective amount of palbociclib, or a pharmaceutically acceptable salt thereof, and a therapeutically effective amount of venetoclax, or a pharmaceutically acceptable salt thereof, in the preparation of a medicament for the treatment of chronic lymphocytic leukemia.

Also provided is the use of a therapeutically effective amount of palbociclib, or a pharmaceutically acceptable salt thereof, and a therapeutically effective amount of trametinib, or a pharmaceutically acceptable salt thereof, in the preparation of a medicament for the treatment of chronic lymphocytic leukemia.

Also provided is the use of a therapeutically effective amount of venetoclax, or a pharmaceutically acceptable salt thereof, and a therapeutically effective amount of trametnib, or a pharmaceutically acceptable salt thereof, in the preparation of a medicament for the treatment of chronic lymphocytic leukemia.

### Treatment.

Disclosed are methods of treating a subject with a hematological malignancy using combinations of compositions described herein. The compounds can be administered by any appropriate route including orally or parenterally including buccally, sublingually, sublabially, by inhalation, intra-arterially, intravenously, intraventricularly, intramuscularly, subcutaneously, intraspinally, intraorbitally, intracranially or intrathecally.

The administration of a pharmaceutical composition including the disclosed compounds can be for prophylactic or therapeutic purposes. For prophylactic and therapeutic purposes, the treatments can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the treatments for viral infection can be provided as repeated doses within a prolonged prophylaxis or treatment regimen that will yield clinically significant results to alleviate one or more symptoms or detectable conditions associated with a disease or condition.

An effective amount or concentration of the disclosed combinations of compounds can be any amount of the two compounds administered by themselves alone or in combination with additional therapeutic agents, is sufficient to achieve a desired effect in a subject. The effective amount of the agent will be dependent on several factors, including the subject being treated and the manner of administration of the compositions. In one example, a therapeutically effective amount or concentration is one that is sufficient to prevent advancement, delay progression, or to cause regression of a disease or condition, or which is capable of reducing symptoms caused by any disease or condition.

In one example, a desired effect is to reduce or inhibit one or more symptoms associated with a disease or condition characterized by hematological malignancy. The one or more symptoms do not have to be completely eliminated for the composition to be effective. For example, a composition can decrease the sign or symptom by a desired amount, for example by at least 20%, at least 40%, at least 50%, at least 80%, at least 90%, at least 95%, at least 98%, or even at least 100%, as compared to how the sign or symptom would have progressed in the absence of the composition or in comparison to currently available treatments.

### Dose Ranges.

The actual effective amount will vary according to factors such as the type of hematological malignancy to be protected against/therapeutically treated and the particular status of the subject (for example, the subject's age, size, fitness, extent of symptoms, susceptibility factors, and the like) time and route of administration, other drugs or treatments being administered concurrently, as well as the specific pharmacology of treatments for hematological malignancy for eliciting the desired activity or biological response in the subject. Dosage regimens can be adjusted to provide an optimum prophylactic or therapeutic response.

An effective amount is also one in which any toxic or detrimental side effects of the compound and/or other biologically active agent is outweighed in clinical terms by therapeutically beneficial effects. An exemplary range for a therapeutically effective amount of treatments for hematological malignancy within the methods and formulations of the disclosure is 0.0001 μg/kg body weight to 10 mg/kg body weight per dose for one or both compounds in the combination, such as 0.0001 μg/kg body weight to 0.001 μg/kg body weight per dose for one or both compounds in the combination, 0.001 μg/kg body weight to 0.01 μg/kg body weight per dose for one or both compounds in the combination, 0.01 μg/kg body weight to 0.1 μg/kg body weight per dose for one or both compounds in the combination 0.1 μg/kg body weight to 10 μg/kg body weight per dose for one or both compounds in the combination, 1 μg/kg body weight to 100 μg/kg body weight per dose for one or both compounds in the combination, 100 μg/kg body weight to 500 μg/kg body weight per dose for one or both compounds in the combination, 500 μg/kg body weight per dose to 1000 μg/kg body weight per dose for one or both compounds in the combination, or 1.0 mg/kg body weight to 10 mg/kg body weight per dose for one or both compounds in the combination.

Quizartinib+Ibrutinib.

Quizartinib may be administered in the methods herein at a dose range of from 1 mg to 75 mg per day in one daily dose or in divided doses. Dose ranges for use in the quizartinib combinations herein include doses of from 1 mg to 50 mg/day, from 1 mg to 10 mg/day, from 5 mg to 15 mg/day, from 10 mg to 20 mg/day, from 15 mg to 25 mg/day, from 20 mg to 30 mg/day, from 25 mg to 35 mg/day, from 30 mg to 40 mg/day, from 35 mg to 45 mg/day, from 40 mg to 50 mg/day, from 45 mg to 55 mg/day, from 50 mg to 60 mg/day, from 55 mg to 65 mg/day, from 60 mg to 70 mg/day, and from 65 mg to 75 mg/day. Specific individual doses of quizartinib for use in the methods herein include 5 mg, 10 mg, 15 mg, 20 mg, 25 mg, 30 mg, 35 mg, 40 mg, and 50 mg doses.

Ibrutinib may be administered in the methods herein at a dose range of from 25 mg to 1,000 mg per day. Dose ranges for use in the ibrutinib combinations herein include doses of from 25 mg to 100 mg/day, from 75 mg to 150 mg/day, from 100 mg to 200 mg/day, from 150 mg to 250 mg/day, from 200 mg to 300 mg/day, from 250 mg to 350 mg/day, from 300 mg to 400 mg/day, from 350 mg to 450 mg/day, from 400 mg to 500 mg/day, from 450 mg to 550 mg/day, from 500 mg to 600 mg/day, from 550 mg to 650 mg/day, from 600 mg to 700 mg/day, from 650 mg to 750 mg/day, from 700 mg to 800 mg/day, and from 750 mg to 850 mg/day, from 800 mg to 900 mg/day, from 850 mg to 950 mg/day, and from 900 mg to 1,000 mg/day. Specific individual doses of ibrutinib for use in the methods herein include 50 mg, 75 mg, 100 mg, 125 mg, 140 mg, 150 mg, 175 mg, 200 mg, 250 mg, 300 mg, 350 mg, 400 mg, 420 mg, 425 mg, 450 mg, 500 mg, 550 mg, 600 mg, 650 mg, 700 mg, 750 mg, 800 mg, 850 mg, 900 mg, 950 mg, and 1,000 mg doses.

Additional combinations of quizartinib and ibrutinib that may be used in the present methods include those in the table below.

| Combination | | |
|---|---|---|
| No. | Quizartinib mg/day | Ibrutinib mg/day |
| 1 | 5 mg-50 mg/day | 50 mg-600 mg/day |
| 2 | 10 mg-45 mg/day | 75 mg-500 mg/day |
| 3 | 15 mg-40 mg/day | 100 mg-450 mg/day |
| 4 | 20 mg-40 mg/day | 100 mg-450 mg/day |
| 5 | 5 mg-30 mg/day | 100 mg-420 mg/day |
| 6 | 5 mg-30 mg/day | 100 mg-350 mg/day |
| 7 | 5 mg-30 mg/day | 75 mg-300 mg/day |
| 8 | 5 mg-30 mg/day | 50 mg-250 mg/day |
| 9 | 5 mg-30 mg/day | 50 mg-200 mg/day |
| 10 | 5 mg-30 mg/day | 50 mg-150 mg/day |
| 11 | 5 mg-30 mg/day | 50 mg-125 mg/day |

Palbociclib.

Palbociclib may be administered in the methods herein at a dose range of from 1 mg to 75 mg per day in one daily dose or in divided doses. Dose ranges for use in the palbociclib combinations herein include doses of from 1 mg to 200 mg/day, from 10 mg to 200 mg/day, from 50 mg to 200 mg/day, from 50 mg to 150 mg/day, from 60 mg to 150 mg/day, from 75 mg to 150 mg/day, from 75 mg to 125 mg/day, from 50 mg to 150 mg/day, from 50 mg to 100 mg/day, from 100 mg to 150 mg/day, from 100 mg to 150 mg/day, and from 100 mg to 200 mg/day. Specific individual doses of palbociclib for use in the methods herein include 1 mg, 5 mg, 10 mg, 15 mg, 20 mg, 25 mg, 30 mg, 35 mg, 40 mg, 50 mg, 60 mg, 70 mg, 75 mg, 80 mg, 90 mg, 100 mg, 110 mg, 120 mg, 125 mg, 150 mg, 160 mg, 175 mg, and 200 mg doses.

Combinations of palbociclib and JQ1 that may be used in the present methods include those in the table below.

| Combination | | |
|---|---|---|
| No. | palbociclib mg/day | JQ1 mg/day |
| 1 | 1 mg-200 mg/day | 20 mg-800 mg/day |
| 2 | 10 mg-200 mg/day | 50 mg-700 mg/day |
| 3 | 25 mg-200 mg/day | 100 mg-450 mg/day |
| 4 | 50 mg-200 mg/day | 200 mg-600 mg/day |
| 5 | 50 mg-150 mg/day | 250 mg-500 mg/day |
| 6 | 25 mg-200 mg/day | 100 mg-350 mg/day |
| 7 | 25 mg-200 mg/day | 100 mg-300 mg/day |
| 8 | 25 mg-200 mg/day | 50 mg-250 mg/day |
| 9 | 25 mg-200 mg/day | 50 mg-200 mg/day |
| 10 | 25 mg-200 mg/day | 50 mg-150 mg/day |
| 11 | 25 mg-200 mg/day | 50 mg-125 mg/day |

Sorafenib.

Sorafenib may be administered in the methods herein at a dose range of from 25 mg to 1,000 mg per day. Dose ranges for use in the sorafenib combinations herein include doses of from 25 mg to 100 mg/day, from 75 mg to 150 mg/day, from 100 mg to 200 mg/day, from 150 mg to 250 mg/day, from 200 mg to 300 mg/day, from 250 mg to 350 mg/day, from 300 mg to 400 mg/day, from 350 mg to 450 mg/day, from 400 mg to 500 mg/day, from 450 mg to 550 mg/day, from 500 mg to 600 mg/day, from 550 mg to 650 mg/day, from 600 mg to 700 mg/day, from 650 mg to 750 mg/day, from 700 mg to 800 mg/day, and from 750 mg to 850 mg/day, from 800 mg to 900 mg/day, from 850 mg to 950 mg/day, and from 900 mg to 1,000 mg/day. Specific individual doses of sorafenib for use in the methods herein include 50 mg, 75 mg, 100 mg, 125 mg, 140 mg, 150 mg, 175 mg, 200 mg, 250 mg, 300 mg, 350 mg, 400 mg, 420 mg, 425 mg, 450 mg, 500 mg, 550 mg, 600 mg, 650 mg, 700 mg, 750 mg, 800 mg, 850 mg, 900 mg, 950 mg, and 1,000 mg doses.

Combinations of sorafenib and JQ1 that may be used in the present methods include those in the table below.

| Combination | | |
|---|---|---|
| No. | sorafenib mg/day | JQ1 mg/day |
| 1 | 50 mg-1,200 mg/day | 20 mg-800 mg/day |
| 2 | 50 mg-1,000 mg/day | 50 mg-700 mg/day |
| 3 | 50 mg-900 mg/day | 100 mg-450 mg/day |
| 4 | 100 mg-800 mg/day | 200 mg-600 mg/day |
| 5 | 150 mg-800 mg/day | 250 mg-500 mg/day |
| 6 | 200 mg-800 mg/day | 100 mg-350 mg/day |
| 7 | 150 mg-600 mg/day | 100 mg-300 mg/day |
| 8 | 150 mg-500 mg/day | 50 mg-250 mg/day |
| 9 | 200 mg-600 mg/day | 50 mg-200 mg/day |
| 10 | 200 mg-500 mg/day | 50 mg-150 mg/day |
| 11 | 200 mg-400 mg/day | 50 mg-125 mg/day |

Ruxolitinib+Cabozantinib.

Ruxolitinib may be administered in the methods herein at a dose range of from 1 mg to 75 mg per day in one daily dose or in divided doses. Dose ranges for use in the ruxolitinib combinations herein include doses of from 1 mg to 200 mg/day, from 1 mg to 150 mg/day, from 5 mg to 200 mg/day, from 5 mg to 150 mg/day, from 1 mg to 120 mg/day, from 1 mg to 100 mg/day, from 5 mg to 125 mg/day, from 5 mg to 100 mg/day, from 5 mg to 80 mg/day, from 10 mg to 150 mg/day, from 10 mg to 125 mg/day, from 10 mg to 100 mg/day, from 1 mg to 75 mg/day, from 1 mg to 60 mg/day, from 1 mg to 50 mg/day, from 1 mg to 40 mg/day, and from 1 to 30 mg/day. Specific individual doses of ruxolitinib for use in the methods herein include 1 mg, 5 mg, 10 mg, 15 mg, 20 mg, 25 mg, 30 mg, 35 mg, 40 mg, 50 mg, 60 mg, 70 mg, 75 mg, 80 mg, 90 mg, 100 mg, 110 mg, 120 mg, 125 mg, 150 mg, 160 mg, 175 mg, and 200 mg doses.

Cabozantinib may be administered in the methods herein at a dose range of from 1 mg to 75 mg per day in one daily dose or in divided doses. Dose ranges for use in the cabozantinib combinations herein include doses of from 1 mg to 200 mg/day, from 1 mg to 150 mg/day, from 5 mg to 200 mg/day, from 5 mg to 150 mg/day, from 1 mg to 120 mg/day, from 1 mg to 100 mg/day, from 5 mg to 125 mg/day, from 5 mg to 100 mg/day, from 5 mg to 80 mg/day, from 10 mg to 150 mg/day, from 10 mg to 125 mg/day, from 10 mg to 100 mg/day, from 1 mg to 75 mg/day, from 1 mg to 60 mg/day, from 1 mg to 50 mg/day, from 1 mg to 40 mg/day, from 35 mg to 75 mg/day, from 40 mg to 60 mg/day, and from 1 to 30 mg/day. Specific individual doses of cabozantinib for use in the methods herein include 1 mg, 5 mg, 10 mg, 15 mg, 20 mg, 25 mg, 30 mg, 35 mg, 40 mg, 50 mg, 60 mg, 70 mg, 75 mg, 80 mg, 90 mg, 100 mg, 110 mg, 120 mg, 125 mg, 150 mg, 160 mg, 175 mg, and 200 mg doses.

Quizartinib+Idelalisib.

Quizartinib can be used in the ranges and doses listed above.

Idelalisib may be administered in the methods herein at a dose range of from 25 mg to 1,200 mg per day. Dose ranges for use in the idelalisib combinations herein include doses of from 25 mg to 100 mg/day, from 75 mg to 150 mg/day, from 100 mg to 200 mg/day, from 150 mg to 250 mg/day, from 200 mg to 300 mg/day, from 250 mg to 350 mg/day, from 300 mg to 400 mg/day, from 350 mg to 450 mg/day, from 400 mg to 500 mg/day, from 450 mg to 550 mg/day, from 500 mg to 600 mg/day, from 550 mg to 650 mg/day, from 600 mg to 700 mg/day, from 650 mg to 750 mg/day, from 700 mg to 800 mg/day, and from 750 mg to 850 mg/day, from 800 mg to 900 mg/day, from 850 mg to 950 mg/day, from 900 mg to 1,000 mg/day, and from 1,000 mg to 1,200 mg/day. Specific individual doses of idelalisib for use in the methods herein include 50 mg, 75 mg, 100 mg, 125 mg, 140 mg, 150 mg, 175 mg, 200 mg, 250 mg, 300 mg, 350 mg, 400 mg, 420 mg, 425 mg, 450 mg, 500 mg, 550 mg, 600 mg, 650 mg, 700 mg, 750 mg, 800 mg, 850 mg, 900 mg, 950 mg, 1,000 mg, and 1,200 mg doses.

Additional combinations of quizartinib and idelalisib that may be used in the present methods include those in the table below.

| Combination | | |
|---|---|---|
| No. | Quizartinib mg/day | Idelalisib mg/day |
| 1 | 5 mg-50 mg/day | 50 mg-1,000 mg/day |
| 2 | 10 mg-45 mg/day | 75 mg-750 mg/day |
| 3 | 15 mg-40 mg/day | 100 mg-700 mg/day |
| 4 | 20 mg-40 mg/day | 100 mg-650 mg/day |
| 5 | 5 mg-30 mg/day | 100 mg-600 mg/day |
| 6 | 5 mg-30 mg/day | 100 mg-500 mg/day |
| 7 | 5 mg-30 mg/day | 50 mg-300 mg/day |
| 8 | 5 mg-30 mg/day | 50 mg-250 mg/day |
| 9 | 5 mg-30 mg/day | 600 mg-1,200 mg/day |
| 10 | 5 mg-30 mg/day | 600 mg-1,000 mg/day |
| 11 | 5 mg-30 mg/day | 150 mg-600 mg/day |
| 12 | 5 mg-30 mg/day | 250 mg-600 mg/day |

Venetoclax+Doramapimod.

Venetoclax may be administered in the methods herein at a dose range of from 1 mg to 1,000 mg per day. Dose ranges for use in the venetoclax combinations herein include doses of from 5 mg to 100 mg/day, from 5 mg to 150 mg/day, from 10 mg to 500 mg/day, from 100 mg to 200 mg/day, from 150 mg to 250 mg/day, from 200 mg to 300 mg/day, from 250 mg to 350 mg/day, from 300 mg to 400 mg/day, from 350 mg to 450 mg/day, from 400 mg to 500 mg/day, from 450 mg to 550 mg/day, from 500 mg to 600 mg/day, from 550 mg to 650 mg/day, from 600 mg to 700 mg/day, from 650 mg to 750 mg/day, from 700 mg to 800 mg/day, and from 750 mg to 850 mg/day, from 800 mg to 900 mg/day, from 850 mg to 950 mg/day, and from 900 mg to 1,000 mg/day. Specific individual doses of venetoclax for use in the methods herein include 1 mg, 5 mg, 10 mg, 20 mg, 30 mg, 40 mg, 50 mg, 75 mg, 100 mg, 125 mg, 140 mg, 150 mg, 175 mg, 200 mg, 250 mg, 300 mg, 350 mg, 400 mg, 420 mg, 425 mg, 450 mg, 500 mg, 550 mg, 600 mg, 650 mg, 700 mg, 750 mg, 800 mg, 850 mg, 900 mg, 950 mg, and 1,000 mg doses.

Doramapimod may be administered in the methods herein at a dose range of from 1 mg to 600 mg per day. Dose ranges for use in the doramapimod combinations herein include doses of from 1 mg to 100 mg/day, from 25 mg to 150 mg/day, from 100 mg to 200 mg/day, from 150 mg to 250 mg/day, from 200 mg to 300 mg/day, from 250 mg to 350 mg/day, from 300 mg to 400 mg/day, from 350 mg to 450 mg/day, from 400 mg to 500 mg/day, from 450 mg to 550 mg/day, and from 500 mg to 600 mg/day. Specific individual doses of doramapimod for use in the methods herein include 1 mg, 5 mg, 10 mg, 20 mg, 30 mg, 40 mg, 50 mg, 75 mg, 100 mg, 125 mg, 140 mg, 150 mg, 175 mg, 200 mg, 250 mg, 300 mg, 350 mg, 400 mg, 420 mg, 425 mg, 450 mg, 500 mg, 550 mg, and 600 mg doses.

Additional combinations of venetoclax and doramapimod that may be used in the present methods include those in the table below.

| Combination | | |
|---|---|---|
| No. | Venetoclax mg/day | Doramapimod mg/day |
| 1 | 5 mg-600 mg/day | 1 mg-600 mg/day |
| 2 | 10 mg-500 mg/day | 5 mg-600 mg/day |
| 3 | 10 mg-450 mg/day | 20 mg-500 mg/day |
| 4 | 10 mg-425 mg/day | 50 mg-500 mg/day |
| 5 | 15 mg-400 mg/day | 100 mg-450 mg/day |
| 6 | 15 mg-300 mg/day | 100 mg-425 mg/day |
| 7 | 20 mg-300 mg/day | 125 mg-400 mg/day |
| 8 | 10 mg-200 mg/day | 50 mg-250 mg/day |
| 9 | 10 mg-100 mg/day | 75 mg-500 mg/day |
| 10 | 100 mg-200 mg/day | 75 mg-400 mg/day |
| 11 | 200 mg-300 mg/day | 100 mg-400 mg/day |
| 12 | 300 mg-450 mg/day | 100 mg-400 mg/day |

Additional combinations of venetoclax and palbociclib that may be used in the present methods include those in the table below.

Venetoclax+Palbociclib.

| Combination | | |
|---|---|---|
| No. | Venetoclax mg/day | Palbociclib mg/day |
| 1 | 5 mg-600 mg/day | 1 mg-200 mg/day |
| 2 | 10 mg-500 mg/day | 5 mg-200 mg/day |
| 3 | 10 mg-450 mg/day | 20 mg-200 mg/day |
| 4 | 10 mg-425 mg/day | 50 mg-200 mg/day |
| 5 | 15 mg-400 mg/day | 50 mg-150 mg/day |
| 6 | 15 mg-300 mg/day | 50 mg-125 mg/day |
| 7 | 20 mg-300 mg/day | 25 mg-100 mg/day |
| 8 | 10 mg-200 mg/day | 50 mg-100 mg/day |
| 9 | 10 mg-100 mg/day | 75 mg-200 mg/day |
| 10 | 100 mg-200 mg/day | 50 mg-150 mg/day |
| 11 | 200 mg-300 mg/day | 50 mg-150 mg/day |
| 12 | 300 mg-450 mg/day | 50 mg-150 mg/day |

Venetoclax+Trametinib.

Trametinib may be administered in the methods herein at a dose range of from 0.1 mg to 5 mg per day. Dose ranges for use in the trametinib combinations herein include doses of from 0.1 mg to 4 mg/day, from 0.1 mg to 3 mg/day, from 0.1 mg to 2.5 mg/day, from 0.1 mg to 2 mg/day, from 0.1 mg to 1.75 mg/day, from 0.1 mg to 1.5 mg/day, 0.5 mg to 4 mg/day, from 0.5 mg to 3 mg/day, from 0.5 mg to 2.5 mg/day, from 0.5 mg to 2 mg/day, from 0.5 mg to 1.75 mg/day, from 0.5 mg to 1.5 mg/day, 0.75 mg to 4 mg/day, from 0.75 mg to 3 mg/day, from 0.75 mg to 2.5 mg/day, from 0.75 mg to 2 mg/day, from 0.75 mg to 1.5 mg/day, 1 mg to 4 mg/day, from 1 mg to 3 mg/day, from 1 mg to 2.5 mg/day, from 1 mg to 2 mg/day, from 1 mg to 1.75 mg/day, and from 1 mg to 1.5 mg/day. Specific individual doses of trametinib for use in the methods herein include 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.25 mg, 1.3 mg, 1.4 mg, 1.5 mg, 1.6 mg, 1.7 mg, 1.75 mg, 1.8 mg, 1.9 mg, 2 mg, 2.25 mg, 2.5 mg doses, 3 mg, 3.5 mg, and 4 mg doses.

Additional combinations of venetoclax and trametinib that may be used in the present methods include those in the table below.

| Combination | | |
|---|---|---|
| No. | Venetoclax mg/day | Trametinib mg/day |
| 1 | 5 mg-600 mg/day | 0.1 mg-5 mg/day |
| 2 | 10 mg-500 mg/day | 0.1 mg-4 mg/day |
| 3 | 10 mg-450 mg/day | 0.1 mg-3 mg/day |
| 4 | 10 mg-425 mg/day | 0.1 mg-2.5 mg/day |
| 5 | 15 mg-400 mg/day | 0.5 mg-3 mg/day |
| 6 | 15 mg-300 mg/day | 0.5 mg-2.5 mg/day |
| 7 | 20 mg-300 mg/day | 0.5 mg-2.5 mg/day |
| 8 | 10 mg-200 mg/day | 0.5 mg-2.5 mg/day |
| 9 | 10 mg-100 mg/day | 0.5 mg-2.5 mg/day |
| 10 | 100 mg-200 mg/day | 0.5 mg-2.5 mg/day |
| 11 | 200 mg-300 mg/day | 0.5 mg-2.5 mg/day |
| 12 | 300 mg-450 mg/day | 0.5 mg-2.5 mg/day |

Combinations of trametinib and palbociclib that may be used in the present methods include those in the table below.

| Combination | | |
|---|---|---|
| No. | Trametinib mg/day | Palbociclib mg/day |
| 1 | 0.1 mg-5 mg/day | 1 mg-200 mg/day |
| 2 | 0.1 mg-4 mg/day | 5 mg-200 mg/day |
| 3 | 0.1 mg-3 mg/day | 20 mg-200 mg/day |
| 4 | 0.1 mg-2.5 mg/day | 50 mg-200 mg/day |
| 5 | 0.5 mg-3 mg/day | 50 mg-150 mg/day |
| 6 | 0.5 mg-2.5 mg/day | 50 mg-125 mg/day |
| 7 | 0.5 mg-2.5 mg/day | 25 mg-100 mg/day |
| 8 | 0.5 mg-2.5 mg/day | 50 mg-100 mg/day |
| 9 | 0.5 mg-2.5 mg/day | 75 mg-200 mg/day |
| 10 | 0.5 mg-2.5 mg/day | 50 mg-150 mg/day |
| 11 | 0.5 mg-2.5 mg/day | 100 mg-150 mg/day |
| 12 | 0.5 mg-2.5 mg/day | 75 mg-150 mg/day |

Combinations of venetoclax and JQ1 that may be used in the present methods include those in the table below.

| Combination | | |
|---|---|---|
| No. | Venetoclax mg/day | JQ1 mg/day |
| 1 | 5 mg-600 mg/day | 20 mg-800 mg/day |
| 2 | 10 mg-500 mg/day | 50 mg-700 mg/day |
| 3 | 10 mg-450 mg/day | 100 mg-450 mg/day |
| 4 | 10 mg-425 mg/day | 200 mg-600 mg/day |
| 5 | 15 mg-400 mg/day | 250 mg-500 mg/day |
| 6 | 15 mg-300 mg/day | 100 mg-350 mg/day |
| 7 | 20 mg-300 mg/day | 100 mg-300 mg/day |
| 8 | 10 mg-200 mg/day | 50 mg-250 mg/day |
| 9 | 10 mg-100 mg/day | 50 mg-200 mg/day |
| 10 | 100 mg-200 mg/day | 50 mg-150 mg/day |
| 11 | 200 mg-300 mg/day | 50 mg-125 mg/day |
| 12 | 300 mg-450 mg/day | 50 mg-150 mg/day |

The combinations disclosed herein may be used with other agents in the treatment of acute myeloid leukemia (AML), including arsenic trioxide, Cerudibine (daunorubicin HCl), Clafen (cyclophosphamide), cytarabine, Cytoxen (cyclophosphamide), daunorubicin HCl and cytarabine, enasidenib mesylate, Idamycin (Idarubucin HCl), ldhifa (enasidenib mesylate), Midostaurin, Mitoxantrone HCl, Neosar (cyclophosphamide), Rydapt (Midostaurin), Tabloid (Thioguanine), Tarabine PFS (Cytarabine), Thioquanine, Vincristine Sulfate, Daunorubicin HCl and Cytarabine Liposome, cladribine, fludarabine, topotecan, etoposide, 6-thioguanine, hydroxyurea, corticosteroids, such as prednisone and dexamethasone, methotrexate, 6-mercaptopurine, azacitidine, and decitabine.

The combinations herein may be used with other agents in the treatment of chronic lymphocytic leukemia (CLL), including alemtuzumab, chlorambucil, ofatumumab, Bendamustine HCl, cyclophosphamide, Fludarabine phosphate, obinutuzumab, Ibrutinib, mechlorethamine HCl, prednisone, rituximab, Fludarabine and rituximab, rituximab and hyaluronidase human, venetoclax, Idelalalisib, Chlorambucil-prednisone, and cyclophosphamide-vincristine sulfate-prednisone (CVP), cyclophosphamide-doxorubicin-vincristine-prednisone (CHOP), Chlorambucil combined with prednisone, reituxumab, obinutuzumab, or ofatumumab, and Pentostatin-cyclophosphamide-rituximab (PCR).

Determination of effective amount is typically based on animal model studies followed up by human clinical trials and is guided by administration protocols that significantly reduce the occurrence or severity of targeted disease or condition symptoms in the subject. Suitable models in this regard include, for example, murine, rat, rabbit, porcine, feline, non-human primate, and other accepted animal model subjects known in the arts. Using such models, only ordinary calculations and adjustments are required to determine an appropriate concentration and dose to administer a therapeutically effective amount of the treatments for hematological malignancies.

In particular embodiments, the methods include treating acute myeloid lymphoma or a myeloproliferative neoplasm, the method by administering a first pharmaceutical composition including venetoclax and a second pharmaceutical composition including Array-382 or doramapimod to the subject. In particular embodiments, the venetoclax is administered at a dose of 5 mg to 500 mg per day, and the doramapimod is administered at a dose of 1 mg to 600 mg per day.

In particular embodiments, the methods include treating acute myeloid lymphoma or a myeloproliferative neoplasm by administering a first pharmaceutical composition including venetoclax and a second pharmaceutical composition including Arry-382 to the subject. In particular embodiments, the venetoclax is administered at a dose of 5 mg to 500 mg per day, and the Arry-382 is administered at a dose of 25 mg to 500 mg per day.

In particular embodiments, the methods include treating chronic lymphoblastic leukemia in a subject, by administering a first pharmaceutical composition including quizartinib and a second pharmaceutical composition including ibrutinib to the subject. In particular embodiments, the quzartinib is at a dose of 5 mg to 50 mg per day, and the ibrutinib is at a dose of 50 mg to 600 mg per day.

In particular embodiments, the methods include treating chronic lymphoblastic leukemia in a subject, by administering a first pharmaceutical composition including JQ1 and a second pharmaceutical composition including sorafenib to the subject. In particular embodiments, the JQ1 is at a dose of 10 mg to 800 mg per day or 20 mg to 800 mg per day, and the sorafenib is at a dose of 50 mg to 1200 mg per day.

In particular embodiments, the methods include treating acute myeloid leukemia in a subject, the method by administering a first pharmaceutical composition including JQ1 and a second pharmaceutical composition including palbociclib to the subject, provided that the acute myeloid leukemia is characterized by a mutation in DNMT3A. In particular embodiments, the JQ1 is administered at a dose of 20 mg to 800 mg per day, and the palbociclib is administered at a dose of 1 mg to 200 mg per day. In particular embodiments, the methods further include detecting the mutation in DNMT3A in a sample from the subject, where the sample is peripheral blood mononuclear cells.

In particular embodiments, the methods include treating myeloid leukemia in a subject by administering a first pharmaceutical composition including JQ1 and a second pharmaceutical composition including sorafenib to the subject, provided that the acute myeloid leukemia is characterized by a mutation in NPM1. In particular embodiments, the JQ1 is administered at a dose of 20 mg to 800 mg per day, and the sorafenib is administered at a dose of 50 mg to 1200 mg per day. In particular embodiments, the methods further include detecting the mutation in NPM1 in a sample from the subject, where the sample is peripheral blood mononuclear cells.

In particular embodiments, the methods include treating acute myeloid leukemia in a subject by administering a first pharmaceutical composition including ruxolitinib and a second pharmaceutical composition including cabozantinib to the subject, provided that the acute myeloid leukemia is characterized by a normal karyotype. In particular embodiments, the ruxolitinib is administered at a dose of 1 mg to 50 mg per day, and the cabozantinib is administered at a dose of 5 mg to 120 mg per day.

In particular embodiments, the methods include treating acute myeloid leukemia in a subject, by administering a first pharmaceutical composition including idelalisib and a second pharmaceutical composition including quizartinib to the subject, provided that the acute myeloid leukemia is characterized by a complex karyotype. In particular embodiments, the idelalisib is administered at a dose of 50 mg to 750 mg per day, and the quizartinib is administered at a dose of 10 mg to 800 mg per day.

In particular embodiments, the methods include treating acute myeloid leukemia in a subject by administering a first pharmaceutical composition including g venetoclax and a second pharmaceutical composition including JQ1 to the subject, provided that the acute myeloid leukemia expresses cell surface CD11b. In particular embodiments, the venetoclax is administered at a dose of 5 mg to 500 mg per day, and the JQ1 is administered at a dose of 10 mg to 800 mg per day. In particular embodiments, the methods further include contacting a sample from the subject with an antibody that binds CD11b, where the sample includes peripheral blood mononuclear cells.

In particular embodiments, the methods include treating acute myeloid leukemia in a subject by administering a first pharmaceutical composition including venetoclax and a second pharmaceutical composition including doramapimod to the subject, provided that the acute myeloid leukemia expresses cell surface CD58. In particular embodiments, the venetoclax is administered at a dose of 5 mg to 500 mg per day, and the doramapimod is administered at a dose of 1 mg to 600 mg per day. In particular embodiments, the methods further include contacting a sample from the subject with an antibody that binds CD58, where the sample includes peripheral blood mononuclear cells. In particular embodiments, the antibody is a label.

In particular embodiments, the methods include treating chronic lymphoblastic leukemia in a subject by administering a first pharmaceutical composition including venetoclax and a second pharmaceutical composition including palbociclib to the subject provided that the subject includes a deletion mutation in chromosome 13q. In particular embodiments, the venetoclax is administered at a dose of 5 mg to 500 mg per day, and the palbociclib is administered at a dose of 25 mg to 200 mg per day.

In particular embodiments, the methods include treating chronic lymphoblastic leukemia in a subject by administering a first pharmaceutical composition including trametinib and a second pharmaceutical composition including palbociclib to the subject; provided that the subject includes a deletion mutation in chromosome 13q. In particular embodiments, the trametinib is administered at a dose of 0.1 mg to 5 mg per day, and the palbociclib is administered at a dose of 25 mg to 200 mg per day.

In particular embodiments, the first pharmaceutical composition and the second pharmaceutical composition are co-formulated.

In particular embodiments, the first pharmaceutical composition and the second pharmaceutical composition are administered at a dose equivalent to the combined IC₅₀, IC₇₀, or IC₉₀ of the first pharmaceutical composition and the second pharmaceutical composition.

### EXAMPLES

The following examples are for illustration only. In light of this disclosure, those of skill in the art will recognize that variations of these examples and other examples of the disclosed invention be possible without undue experimentation.

Freshly isolated primary mononuclear cells from patients with various hematologic malignancies (n=122) were cultured in the presence of a panel of 48 drug combinations in equimolar dose series. The drug combinations were formed with different classes of compounds including kinase inhibitors, bromodomain inhibitors, BH3 mimetics, and histone deacetylase inhibitors. For comparison, cells were also tested against graded concentrations of each inhibitor alone, and sensitivity was assessed by MTS-based viability assay after three days. The efficacy of each combination relative to its respective single agents was calculated as a Combination Ratio (CR) value, defined as the combination IC₅₀ or AUC divided by the lowest single agent IC₅₀ or AUC value. By this metric, a CR value less than 1 indicates the combination is more effective than the most effective single agent. This CR value was derived due to known limitations of applying conventional Chou-Talalay-based synergy calculations (Chou T C et al, Pharmacol Rev 58, 621-681 (2006); incorporated by reference herein) in the context of one or more single drug that is occasionally completely ineffective.

For the screening platform, a measure independent of the model assumption was used. The Combination Ratio (CR) is calculated as the combination effect (IC₅₀ or AUC) divided by the better of two single agent effects. This definition is synonymous with Highest Single Agent effect measure (Lehar, Mol Syst Biol 2007; Geary, Am J Physiol Endocrinol Metab 2013).

To relate these findings with other definitions of synergy, two comparative analyses were performed. First, CR effect measures were correlated for a subset of combinations and samples with Excess Over Bliss (EOB) determinations and observed a high level of agreement between the two methods (Spearman r >0.9; p<0.0001). Second, candidate combinations were evaluated using an expanded analysis of each drug alone over a 7-point dose series and all 49 possible combinations of the two agents on a panel of human leukemia cell lines. Overall, the IC₅₀ and AUC CR measures (determined by equimolar series) correlated well with the Bliss synergy score across the full matrix of combinations (Spearman r=0.649 and 0.787, respectively; p<0.0001).

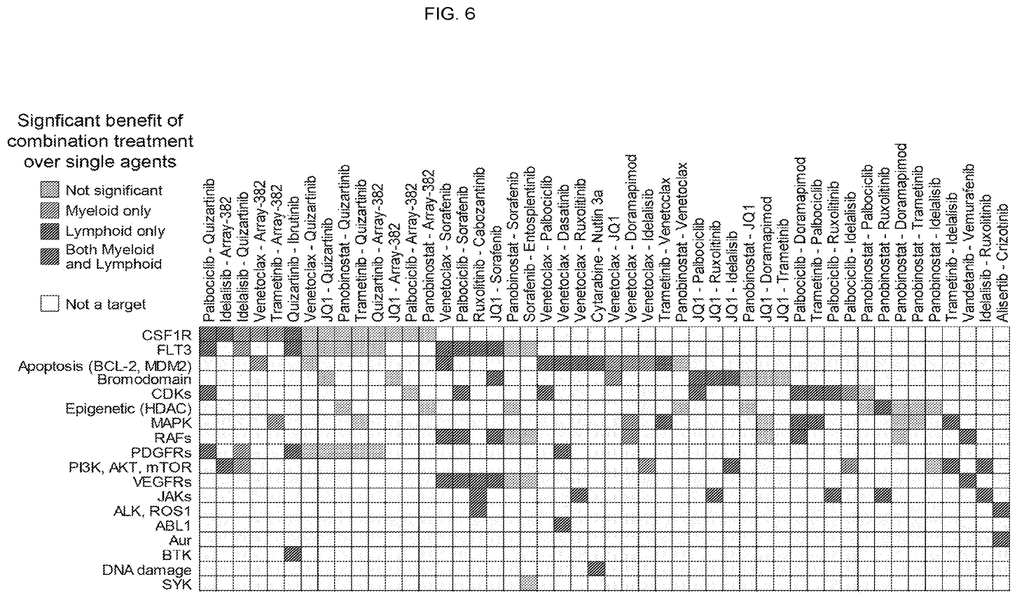

To address the reproducibility of these results, an independent, validation dataset (n=151) of a similar distribution of prospectively collected primary leukemia patient samples was compiled. There is a high level of correlation for both the median IC₅₀ and AUC CRs between the discovery and validation datasets for all combinations and samples tested (Spearman r=0.975 and 0.949, respectively; p<0.0001) (FIG. 6B). Given the particularly notable efficacy of combinations involving venetoclax in AML patient specimens from the discovery cohort, a subset of the most effective of these combinations was compared with those of the validation set AML samples, revealing similar sensitivity distributions in both groups (FIG. 6C). Additionally, expanded synergy analysis of this same subset of combinations was performed for each drug alone over a 7-point dose series and all 49 possible combinations of the two agents on a panel of human leukemia cell lines (MOLM-13, MOLM-14, HL-60, OCI-AML2, OCI-AML3). Overall, cell line IC₅₀ and AUC CR measures (determined by equimolar series) correlated well with the Bliss synergy score across the full matrix of combinations (Spearman r=0.649 and 0.787, respectively; p<0.0001).

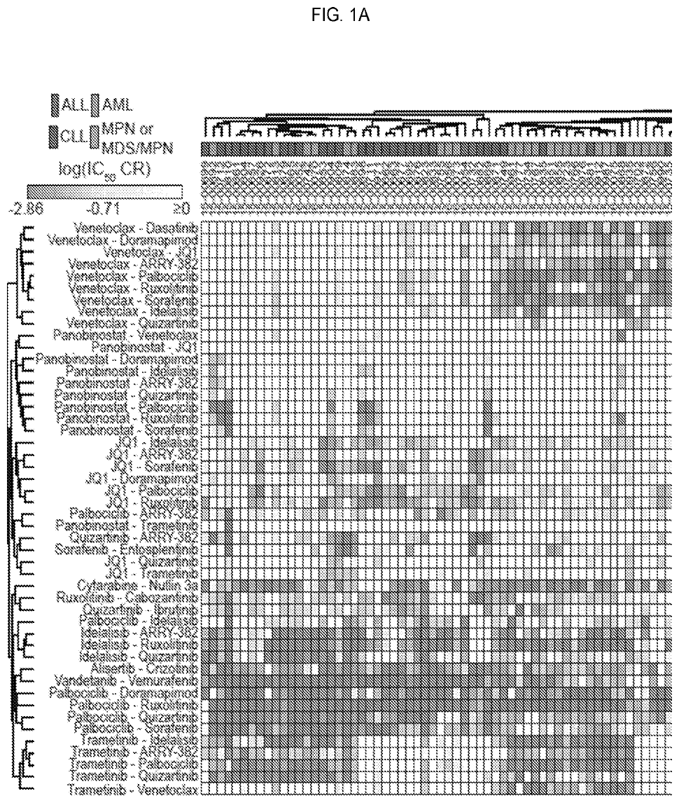

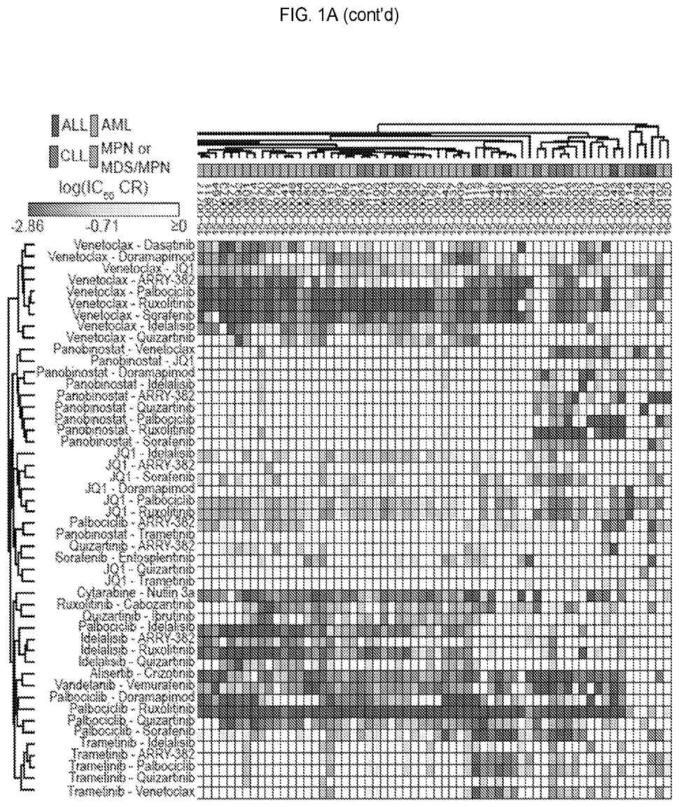

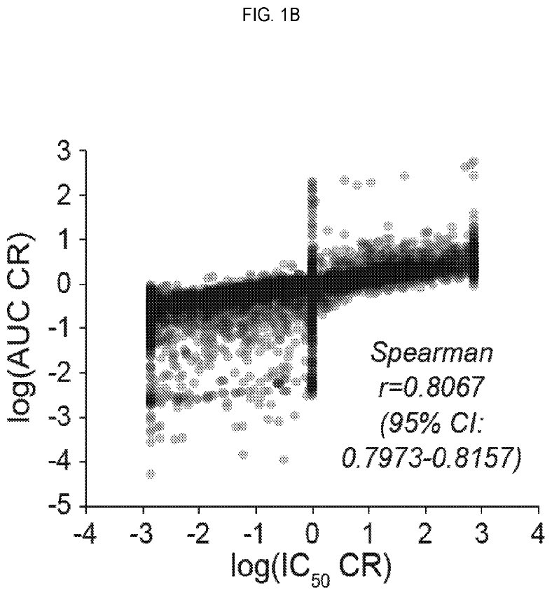

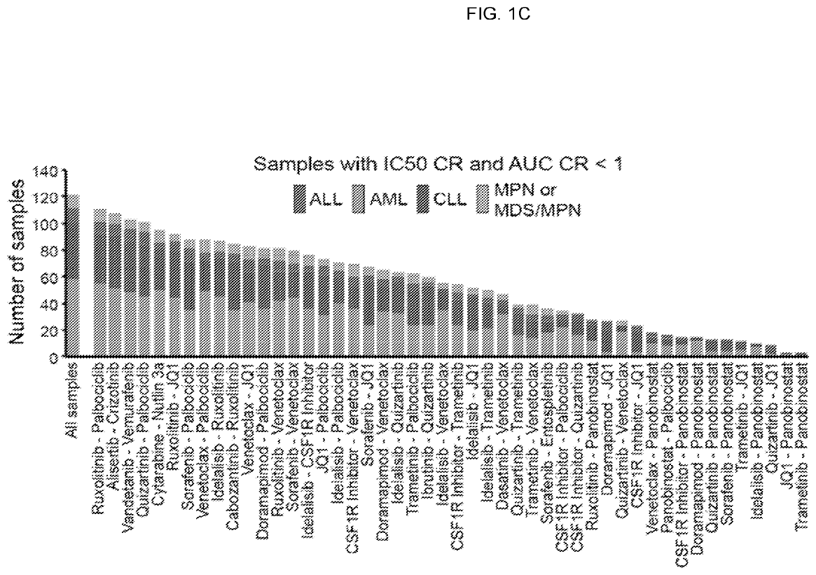

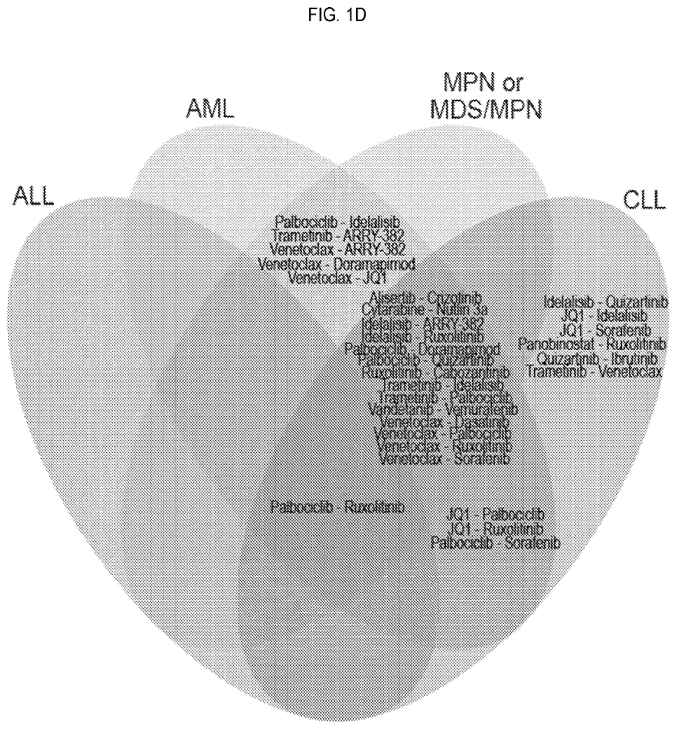

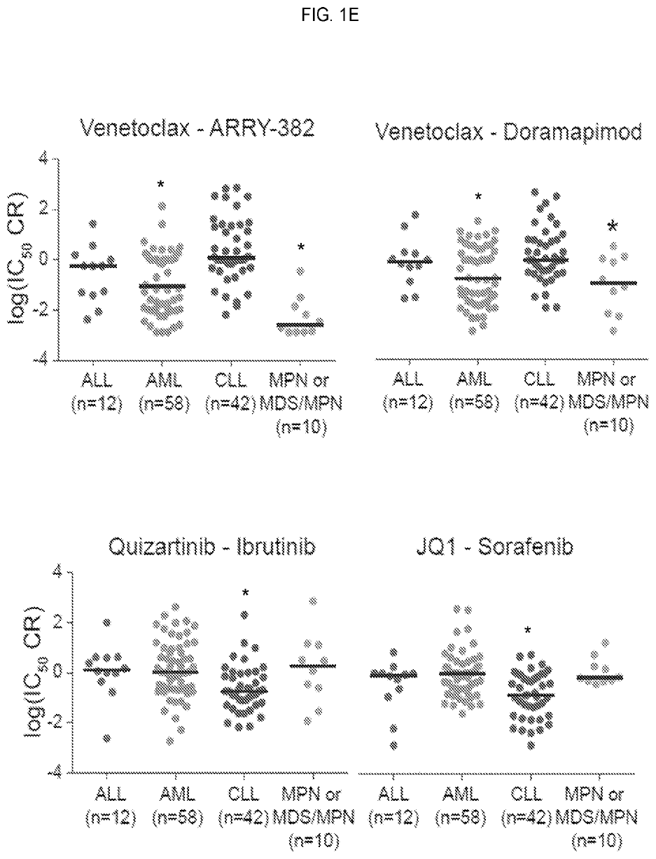

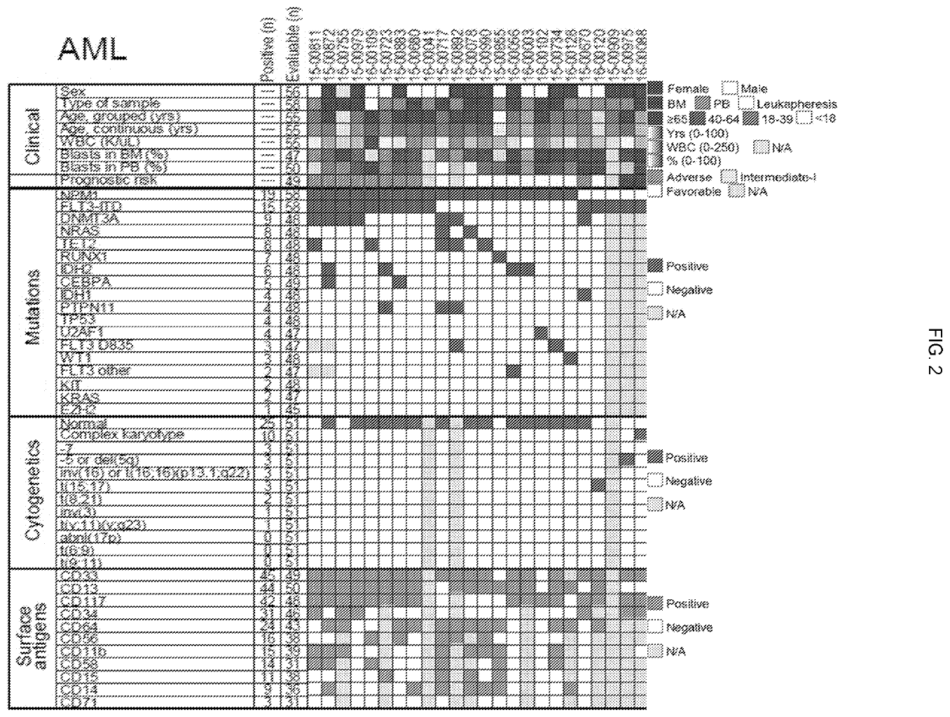

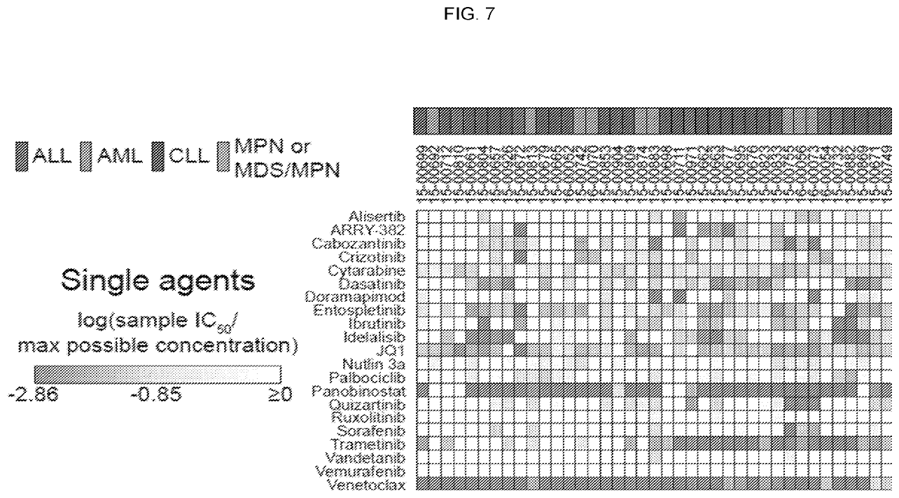

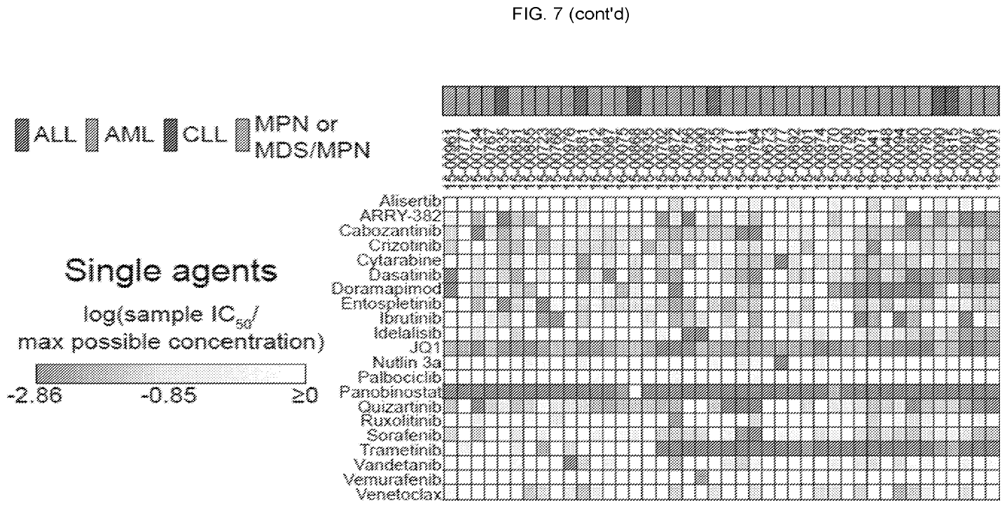

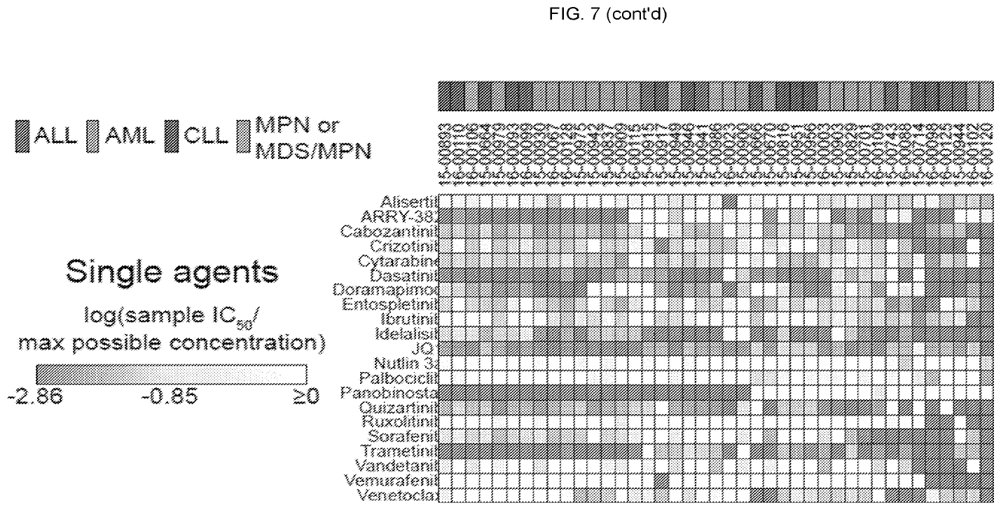

Patients were classified according to four general diagnostic groups: AML, chronic lymphocytic leukemia (CLL), acute lymphoblastic leukemia (ALL), and myeloproliferative neoplasms or myelodysplastic syndromes (MPN or MDS/MPN). Unsupervised hierarchical clustering of CR values for each drug combination revealed several distinct patterns of efficacy (FIG. 1A). Myeloid leukemia patient samples were enriched within a cluster of sensitivity to combinations pairing the Bcl-2 inhibitor, venetoclax, with select kinase inhibitors (dasatinib (multi-kinase), doramapimod (p38), sorafenib (multi-kinase), or idelalisib (PI3KCD)). A subset of samples within this cluster showed sensitivity to combinations involving the MEK inhibitor trametinib and a second kinase inhibitor (idelalisib (PIK3CD), palbociclib (CDK4/6), or quizartinib (FLT3/CSF1R). In contrast, a discrete subcluster of predominantly lymphoid leukemia patients showed sensitivity to combinations of the histone deacetylase inhibitor, panobinostat, in tandem with either the JAK inhibitor, ruxolitinib, or the multi-kinase inhibitor, sorafenib. To ensure that these clusters were due to the effectiveness of the combination rather than that of a single agent, IC₅₀ values for each single agent were mapped according to the combination cluster pattern. Importantly, apart from venetoclax which as a single agent demonstrated potent and selective efficacy in lymphoid (predominantly CLL) patient samples, single agent efficacies did not align uniquely to a combination efficacy-derived, myeloid- or lymphoid cluster FIG. 7).

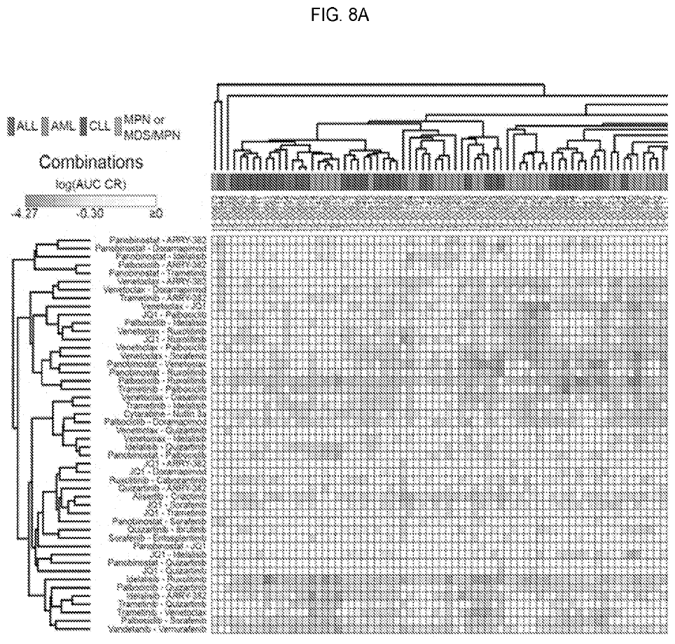

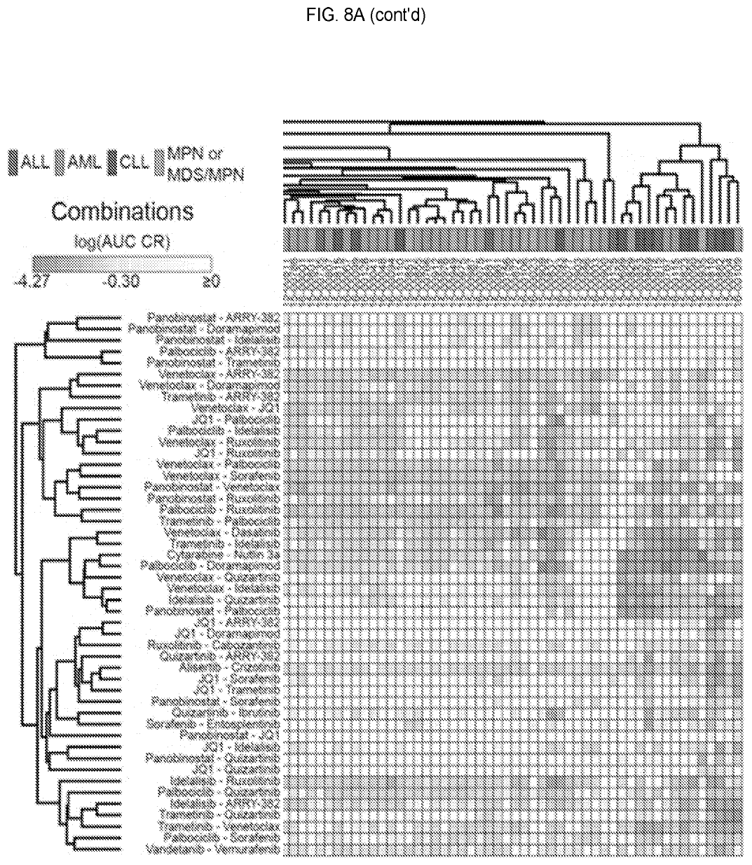

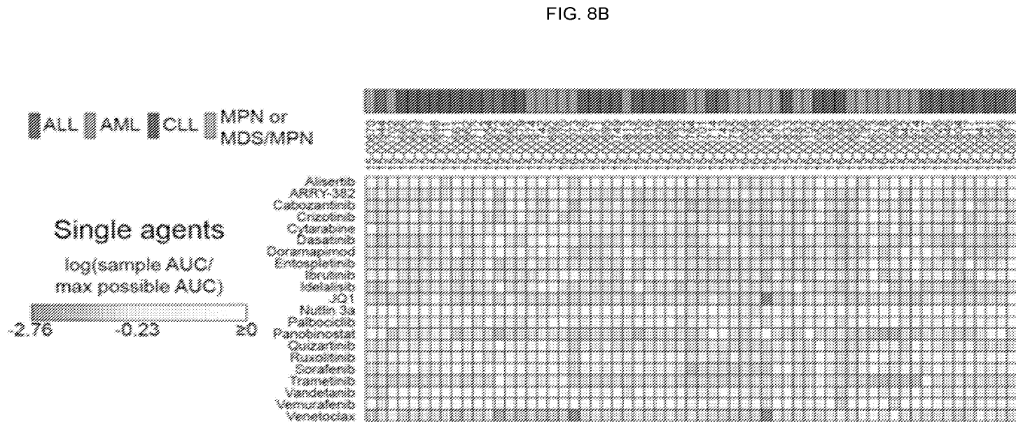

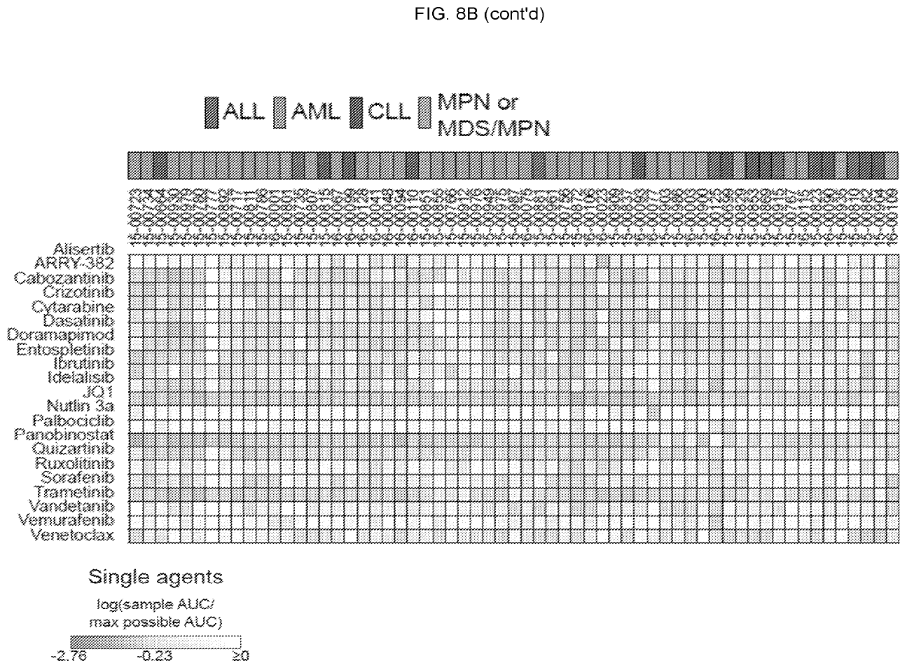

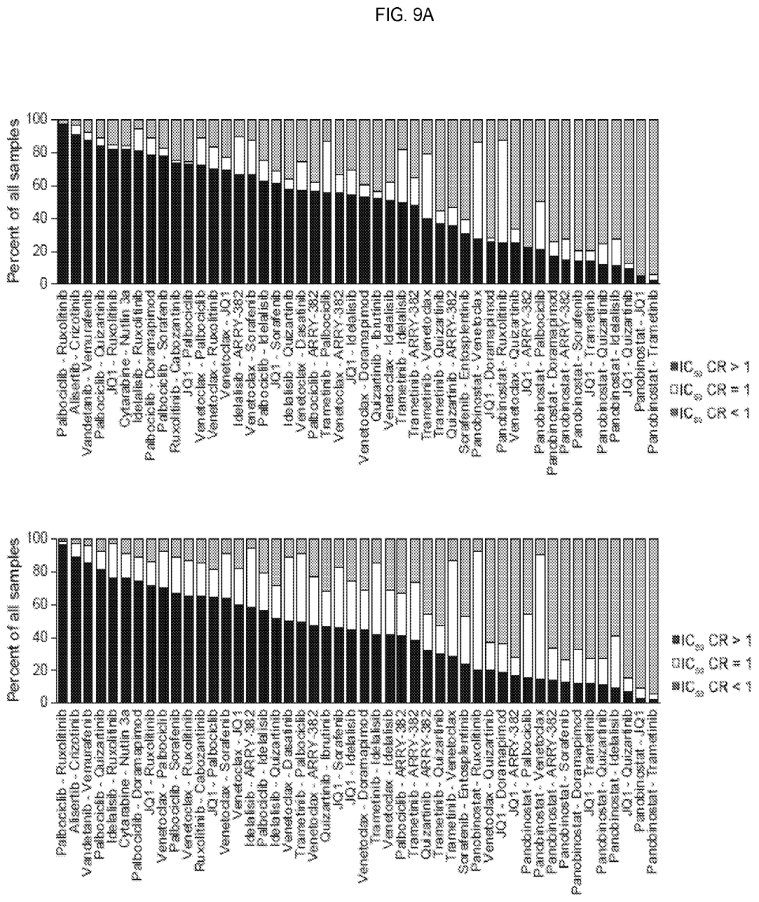

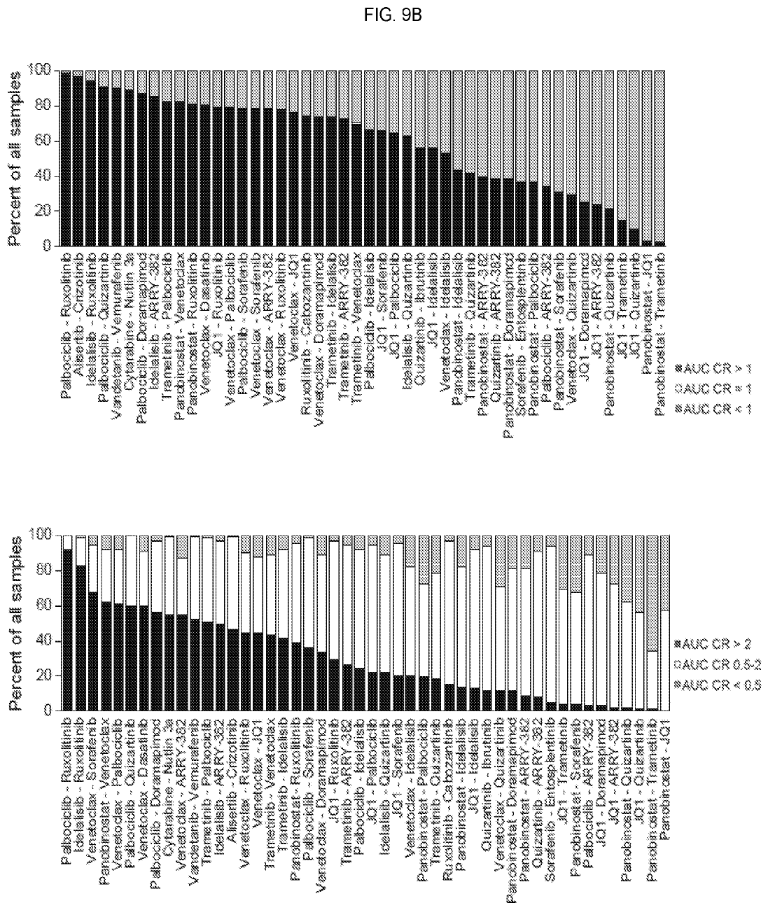

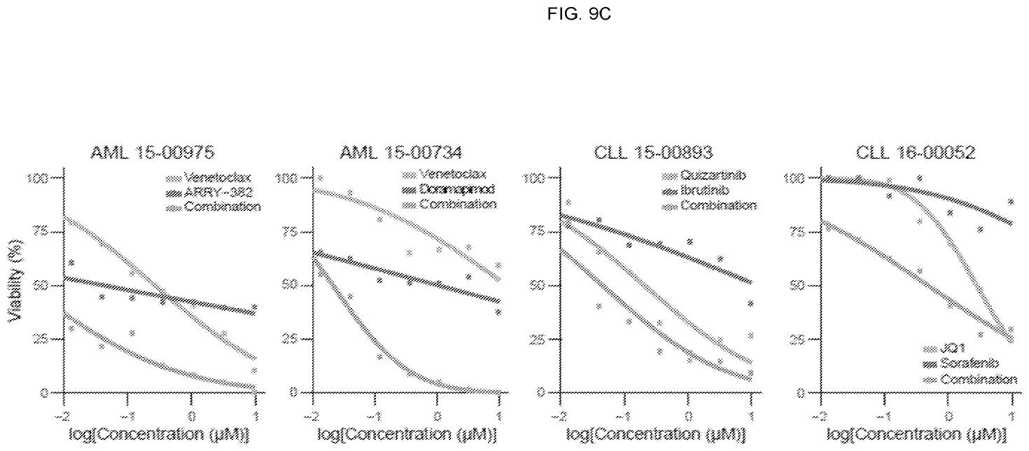

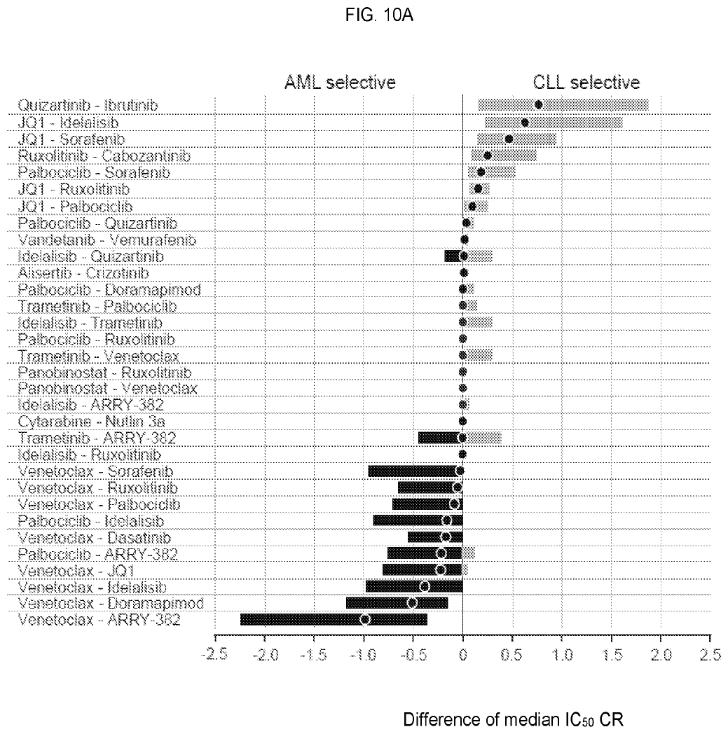

To enable comparison among different measures of efficacy, AUC values were also calculated for each drug-sample pair, and good agreement was observed between matched IC₅₀ and AUC values (Spearman p=0.8131; FIG. 1B), and clustering of AUC CR values yielded similar sensitivity clusters to those seen with IC₅₀ CR results (FIG. 8). In addition, there was a broad distribution in the frequency and type of samples demonstrating combination efficacy. For instance, among samples in which both effect measures produced CR values less than 1, palbociclib-ruxoltinib, alisertib-crizotinib, vandetanib-vemurafenib, and quizartinib-palbociclib were effective by this measure in 100 or more of all 122 patient samples surveyed irrespective of diagnosis. In comparison, several combinations involving the HDAC inhibitor panobinostat showed combination benefit in fewer than 20 patient samples (FIGS. 1C, 9A, 9B). To relate these findings with other definitions of synergy, IC₅₀ and AUC CR effect measures for a subset of combinations and samples were compared with Excess Over Bliss (EOB) determinations (Foucquier and Guedj, 2015). A high level of agreement was observed between the two methods (Spearman r for IC₅₀ CR or AUC CR vs EOB: 0.953 and 0.928, respectively; p<0.0001).

To determine combinations that were more frequently effective within specific diagnostic categories, the median IC₅₀/AUC CR values for each drug within each of the four diagnostic categories were compared to a reference value of CR equal to 1. Consistent with findings from clustering of CR values, several venetoclax-inclusive combinations exhibited median CR values significantly less than 1 selectively among patient samples with myeloid malignancies (FIG. 1D). Pairings of venetoclax with idelalisib or dasatinib were preferentially effective for AML exclusively, while combinations of venetoclax and the CSF1R inhibitor Arry-382, MAPK inhibitor doramapimod, or bromodomain inhibitor JQ1 demonstrated significant benefit more broadly among all myeloid malignancy patients. In contrast, five combinations were selectively effective in CLL patient samples only, including quizartinib-ibrutinib and JQ1-sorafenib (FIG. 1E). Several combinations were significantly effective beyond either single agent in both myeloid and lymphoid samples, with the most common broad efficacy attributable to combinations of the CDK4/6 inhibitor, palbociclib, with either ruxolitinib or quizartinib. While no unique effective combinations were identified for ALL patient samples, this was likely attributable to its smaller cohort size (n=12) among diagnostic categories surveyed. Taken together, these findings suggest patterns of selective efficacy of targeted therapy combinations among disease types, including opportunities for efficacy across heterogeneous diagnostic subtypes of myeloid and lymphoid malignancies.

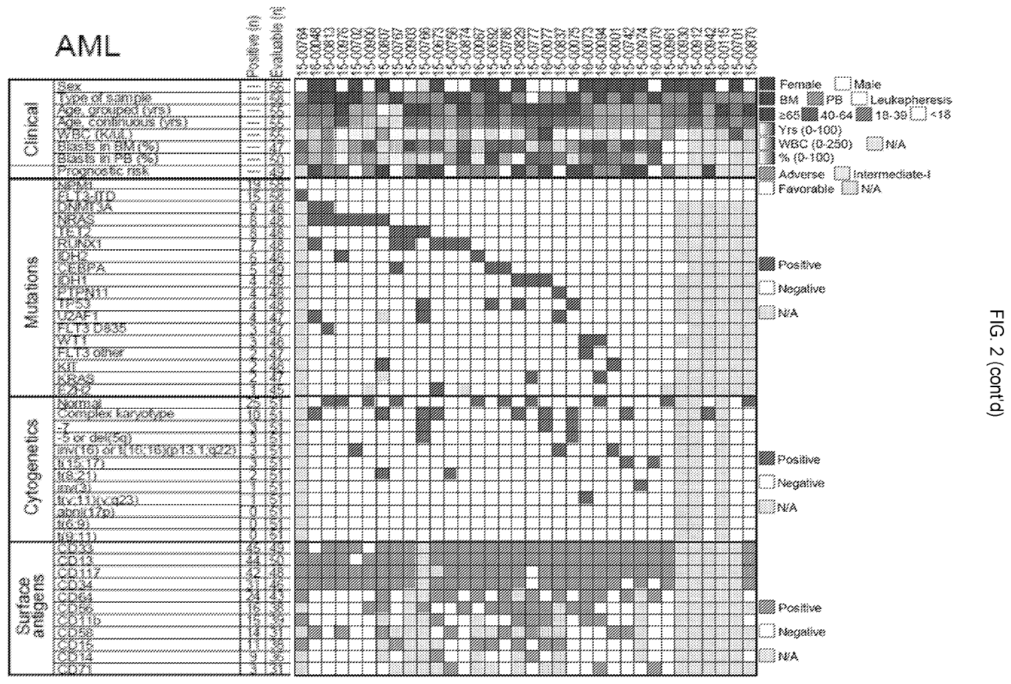

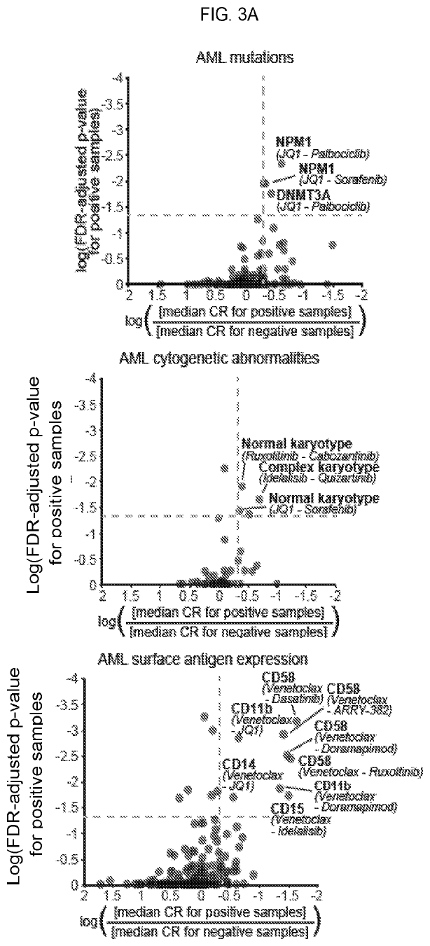

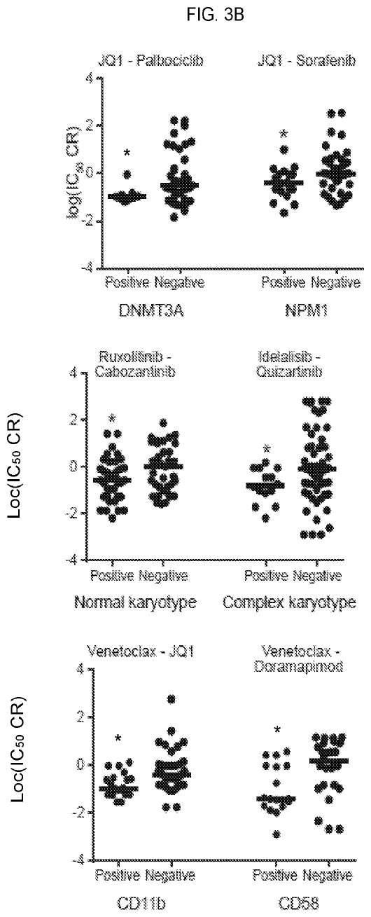

For the two largest diagnostic groups, AML and CLL, expanded panels of clinical, prognostic, mutational, cytogenetic, and surface antigen data were compiled for comparisons according to CR values for each combination. For AML samples (n=58), beyond general clinical characteristics such as age, sex, and WBC count, additional annotations examined included mutational profiling using a focused panel of genes commonly mutated in AML, along with cytogenetic features and cell surface antigen expression as determined by standard chromosome analysis and flow cytometry (FIG. 2). Notably, the most prevalent mutation in this cohort was NPM1 (34%), and 50% of the cohort featured a normal karyotype. Patients harboring mutations in NPM1 demonstrated significantly enhanced sensitivity to combinations of JQ1 and sorafenib (median CR: 0.357). Patients harboring mutations in DNMT3A demonstrated significantly enhanced sensitivity to JQ1 and palbociclib (median CR: 0.119) (FIGS. 3A, 3B). Patients with normal cytogenetics were sensitive to combinations of ruxolitinib-cabozantinib and JQ1-sorafenib, whereas those harboring complex karytopes were significantly sensitive to the combination of idelalisib and quizartinib (FIGS. 3A, 3B; middle panel). Surface expression of several specific myeloid markers was also associated with significant sensitivity to combinations involving venetoclax, including CD11b (Integrin αM) and CD58 (LFA-3) to combinations of venetoclax and JQ1 or venetoclax and doramapimod, respectively (FIGS. 3A, 3B; bottom panel). For AML samples (n=58), beyond general clinical characteristics such as age, sex, and white blood cell (WBC) count, additional annotations examined included mutational profiles from a focused panel of genes commonly mutated in AML, cytogenetic features from standard chromosome analysis, and cell surface antigen expression according to flow cytometry (FIG. 2). Notably, the most prevalent mutation in this cohort was NPM1 (33%), and 50% of the cohort featured normal karyotype. Patients harboring mutations in NPM1 demonstrated significantly enhanced sensitivity to the JQ1-sorafenib combination (median IC₅₀ CR: 0.437; FDR-adjusted p=0.010).

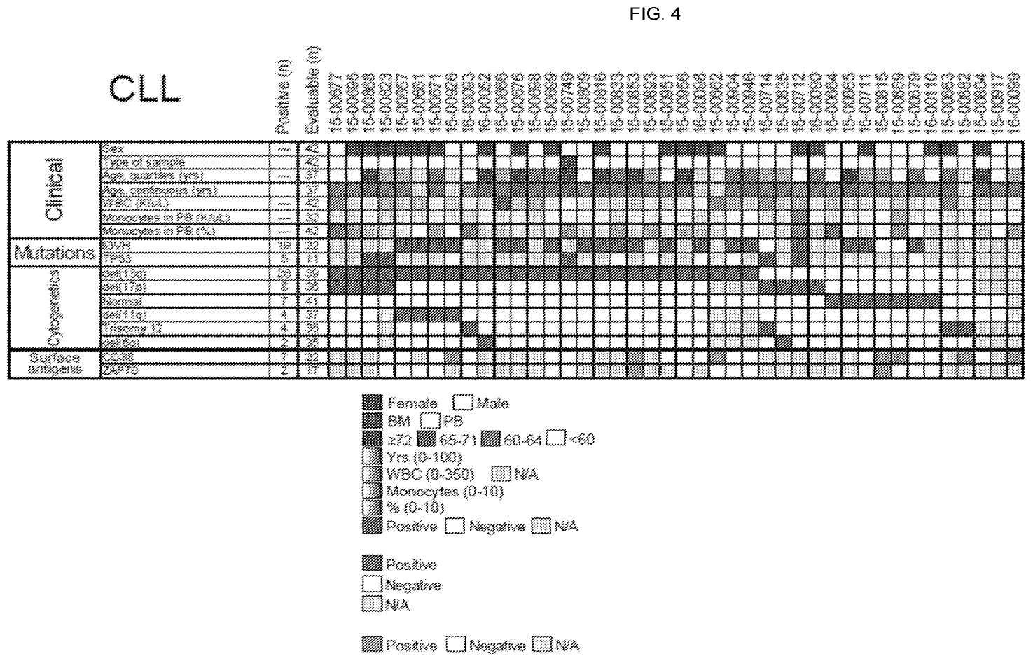

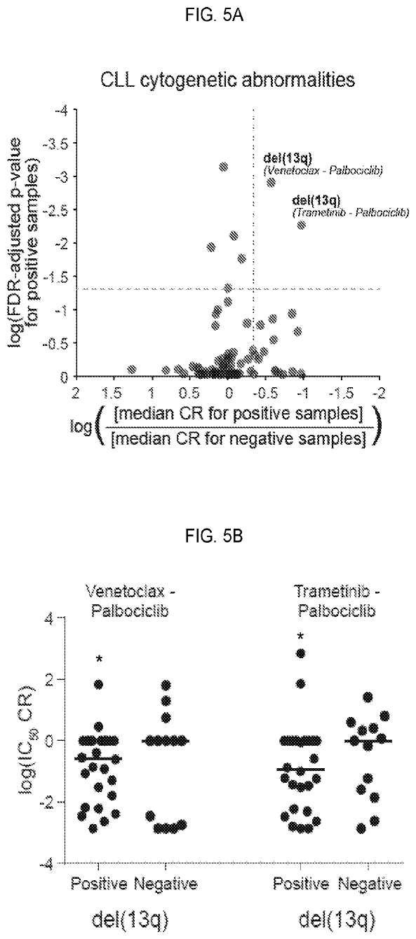

CLL samples were characterized for the mutational status of IgV_{H} and TP53. Cytogenetic features for chromosomal deletions and trisomy as well as prognostic cell surface antigens, CD38 and ZAP70, were determined by standard chromosome analysis and flow cytometry, respectively (FIG. 4). Among the tested combinations, the most significant associations with respect to the disease characteristics were observed in patients harboring deletion of 13q, who showed significant sensitivity to combinations of palbociclib with either venetoclax or trametinib (median CR: 0.267 and 0.116, respectively; FIGS. 5A, 5B).

Taken together, these results implicate multiple potential opportunities for application of combination therapies in distinct diagnostic, genetic/cytogenetic, and phenotypic subsets of patients with AML and CLL.

As the vast molecular heterogeneity of cancer continues to be unraveled, the necessity of defining actionable targeted therapeutic strategies for patients remains paramount to improvement in outcomes. Given difficulties involving the often non-durable responses and rapid development of resistance observed with many single-agent targeted therapies, effective combination strategies for patients with hematologic malignancies are needed. Prior to the work described herein, over 1000 primary patient specimens were screened against a panel of single-agent small-molecule inhibitors. Using these historical drug sensitivity data, drugs were ranked by IC₅₀. These rankings were then used to assemble an initial panel of drug combinations including kinase inhibitors with non-overlapping pathways. Primary patient samples with various hematologic malignancies were screened. Based on data from this initial panel, a second iteration including new combinations of kinase inhibitors as well as combinations of inhibitors from select additional drug classes was generated. For both panels, broad cytotoxicity was problematic for many combinations, and unsupervised hierarchical clustering of CR values revealed no distinct clusters tracking with diagnosis category. This preliminary work informed the design of the panel of combinations described herein. The disclosed panel includes several additional classes of inhibitors as well as a focused inclusion of FDA-approved drugs or drugs in clinical development wherever possible. To this end, defining optimal drug combinations for various malignancies is a highly iterative process of refinement. While logistical considerations preclude comprehensive evaluation of all possible pairwise-combinations of inhibitors, these cumulative data may also prove amenable to applied machine-learning based computational models to predict novel critical drug target pairings in specific malignancies as well as be adaptable as a testing scheme for other tumor types given recent developments to isolate circulating tumor cells.

A critical finding in this study is the effectiveness of several combinations of targeted agents that include a kinase inhibitor and venetoclax, a selective inhibitor of Bcl-2, for myeloid-derived tumors (FIG. 6). These combinations can be identified in the disclosed ex vivo assay where inhibition of a kinase-derived proliferative signal with a specific inhibitor coupled with an anti-apoptotic agent augments efficacy. Combinations such as dasatinib, doramapimod, sorafenib, or idelasilib combined with venetoclax are broadly effective on myeloid-derived tumor samples, and might be useful for treatment of AML in particular. Collectively, these combinations appear to be effective across a broad percentage of AML patient samples, irrespective of cohort subtype heterogeneity. Consistent with these observations, recent reports indicate the combined inhibition of BCR-ABL1 and Bcl-2 is a promising strategy for targeting Philadelphia chromosome-positive ALL as well as the stem cell population in chronic myeloid leukemia (Leonard J T et al, Sci Transl Med 8, 354 ra114 (2016) and Carter B Z et al, Sci Transl Med 8, 355ra117 (2016); both of which are incorporated by reference herein). Certain combinations with venetoclax were also observe to be effective on CLL samples with 13q deletions. Although venetoclax has recently achieved FDA-approval for CLL patients with 17p deletions, the data disclosed herein indicate that venetoclax even as a single agent might be more broadly effective in CLL patients with diverse cytogenetic profiles, and combinations may offer options particularly on disease states where venetoclax as a single agent is not effective. It is noteworthy that venetoclax is effective for a variety of hematologic malignancy subsets including chronic lymphocytic leukemia, multiple myeloma, and acute myeloid leukemia (Davids M S et al, Leuk Lymphoma 54, 1823-1825 (2013); Pan R et al, Cancer Discov 4, 362-375 (2014); Anderson M A et al, Blood 127, 3215-3224 (2016); Roberts A W et al, N Engl J Med 374, 311-322 (2016); all of which are incorporated by reference herein.

Developments in screening of ex vivo cells have produced several assay platforms for evaluating responses of tumor cells to exogenous perturbations. Such screening efforts in hematologic malignancies have often involved the culture of patient cells in conventional two-dimensional tissue culture platforms, sometimes with conventional culture conditions (Tyner J W et al, Proc Natl Acad Sci USA 106, 8695-8700 (2009); Pemovska T et al, Cancer Discov 3, 1416-1429 (2013); and Tyner J W et al, Cancer Res 73, 285-296 (2013); all of which are incorporated by reference herein and other times using additives or feeder cell co-culture that promote certain phenotypic aspects of the cells, such as preservation of primitive cell differentiation state (Pabst C et al, Nat Methods 11, 436-442 (2014); incorporated by reference herein) and cell proliferation (Klco J M et al, Blood 121, 1633-1643 (2013); incorporated by reference herein). A key feature of the disclosed ex vivo assay is that it provides drug sensitivity data within 4 days, a time frame that can support and influence clinical decision-making. Furthermore, this approach addresses some of the challenges in deploying effective therapies where there is a substantial gap between clinical, diagnostic, or genetic markers and available drugs. Ideally the integration of both functional and genomic data types might facilitate a more precise insight into the molecular mechanisms contributing to disease. For example, the disclosed data suggest that combined targeting of the BTK inhibitor ibrutinib and the multikinase inhibitor quizartinib may represent a promising strategy for patients with CLL. Ibrutinib's efficacy is well established for CLL (reviewed in Maddocks K and Jones J A, Semin Oncol 43, 251-259 (2016); incorporated by reference herein). While quizartinib is primarily considered to be a FLT3 inhibitor, it is also a potent inhibitor of CSF1R, a target implicated for the treatment of this disease recently due to its effects on supportive nurse-like, monocyte/macrophage cells that express CSF1R (Galletti G et al, Leukemia doi 10.1038/Ieu.2016.261 (2016) and Galletti G et al, Cell Rep 14, 1748-1760 (2016); both of which are incorporated by reference herein. Accordingly, simultaneous inhibition of both BTK- and CSF1R-mediated signaling pathways may result in further improvement of responses (FIG. 6).

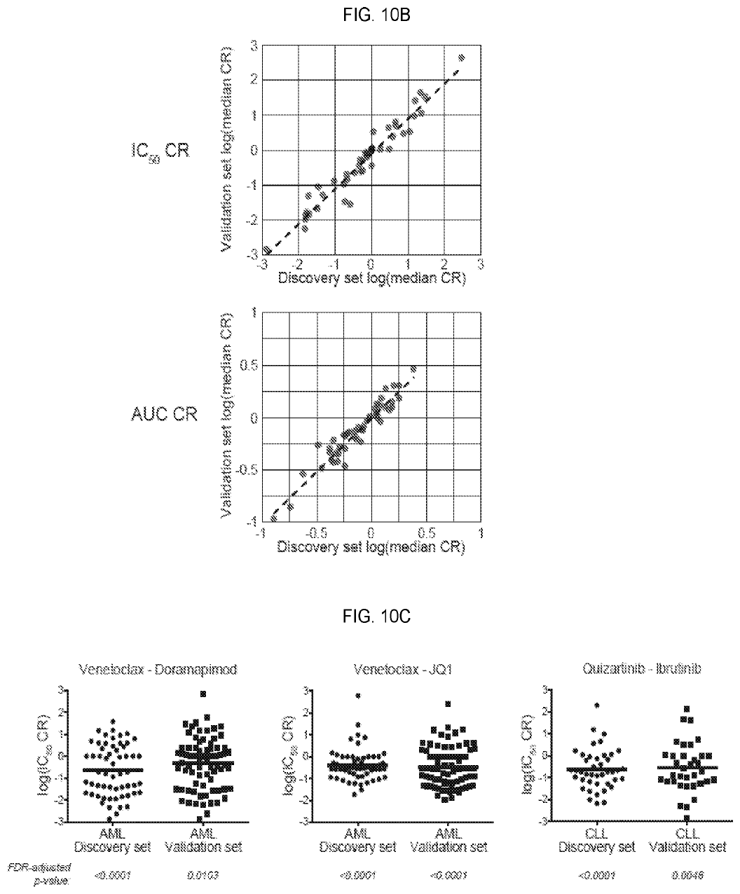

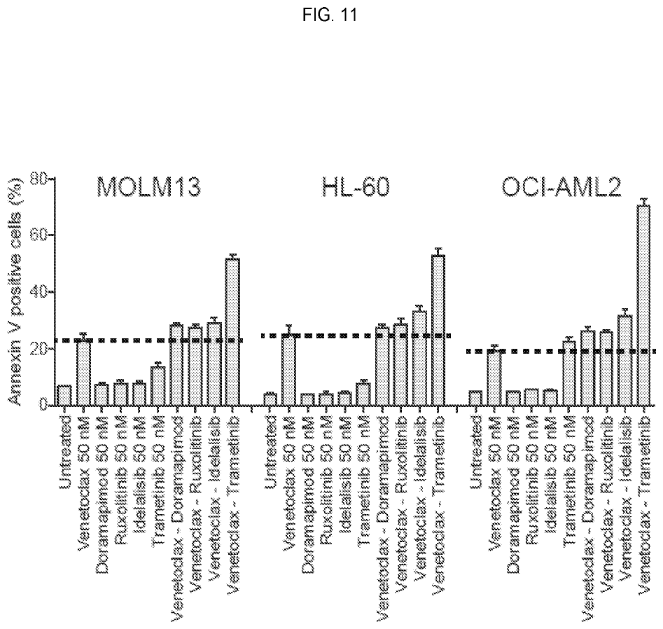

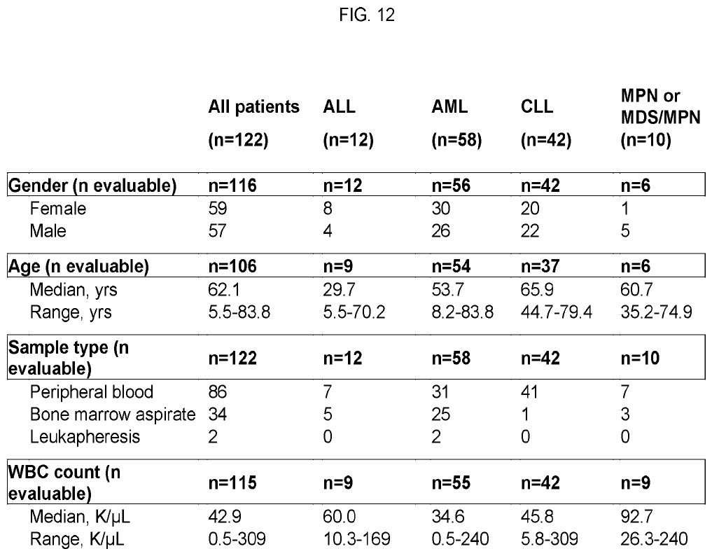

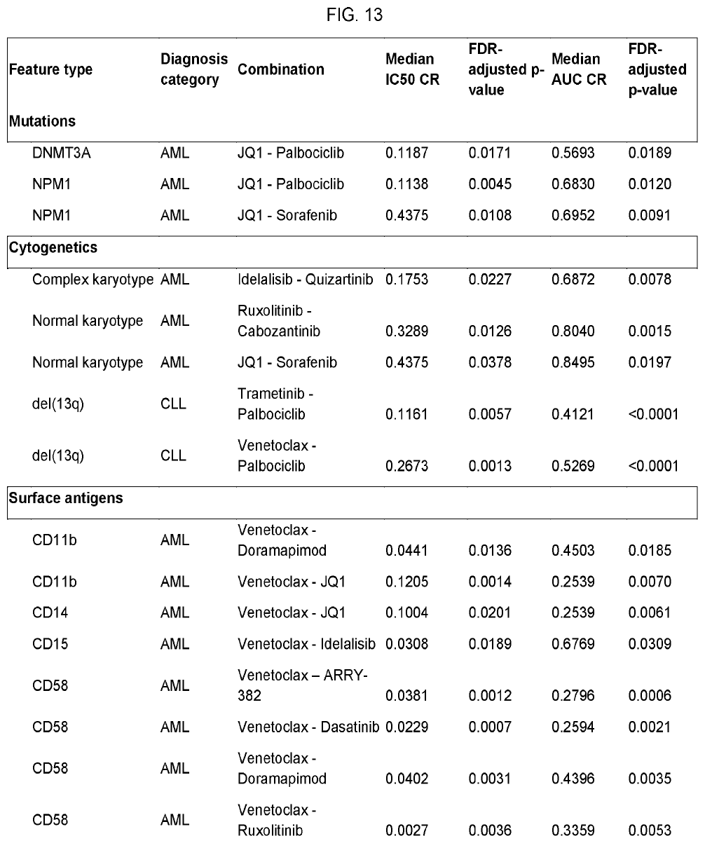

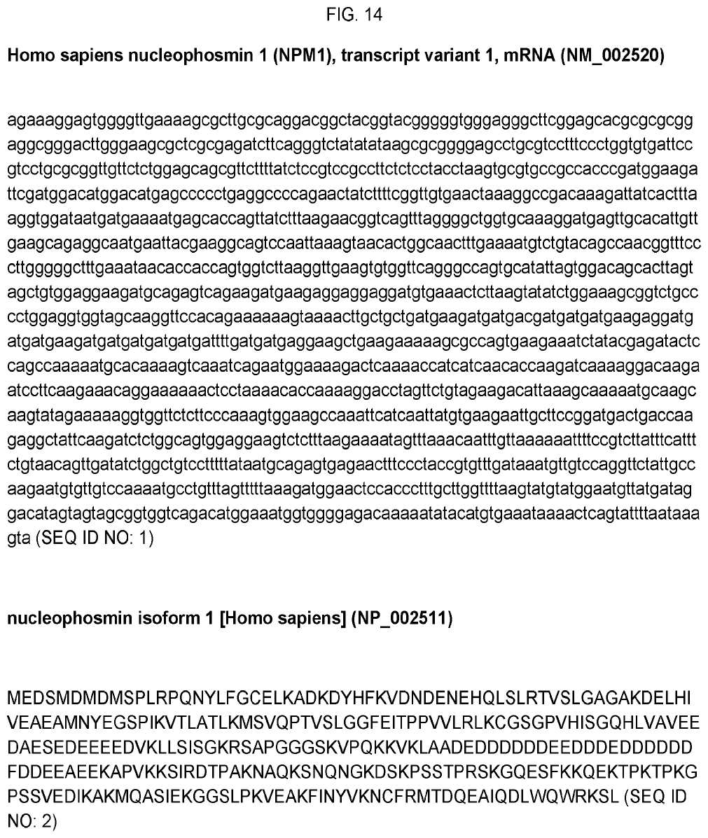

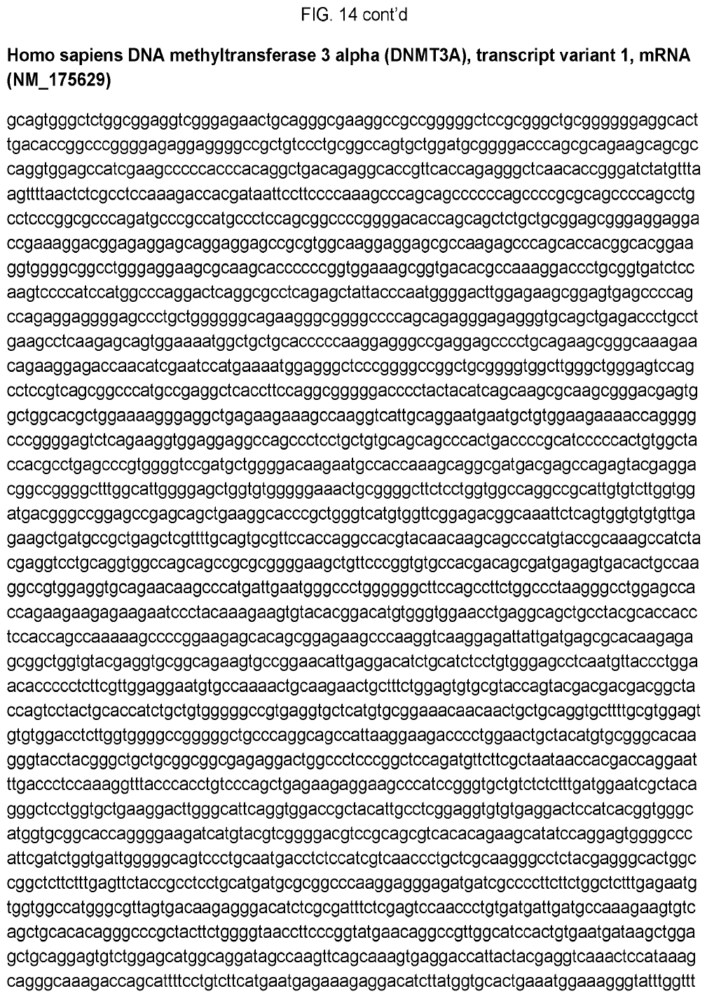

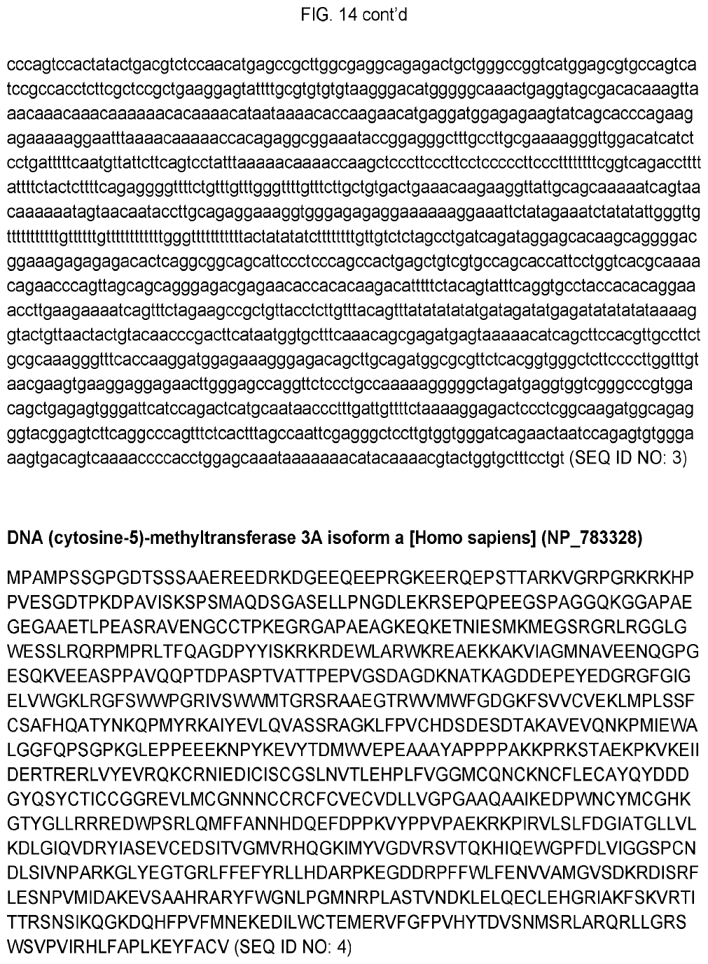

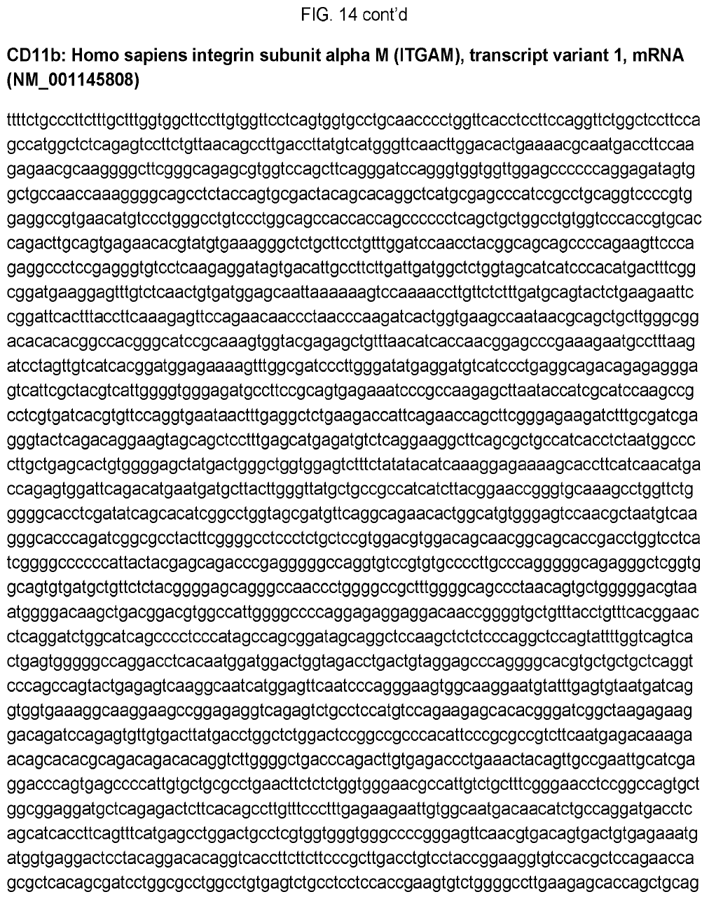

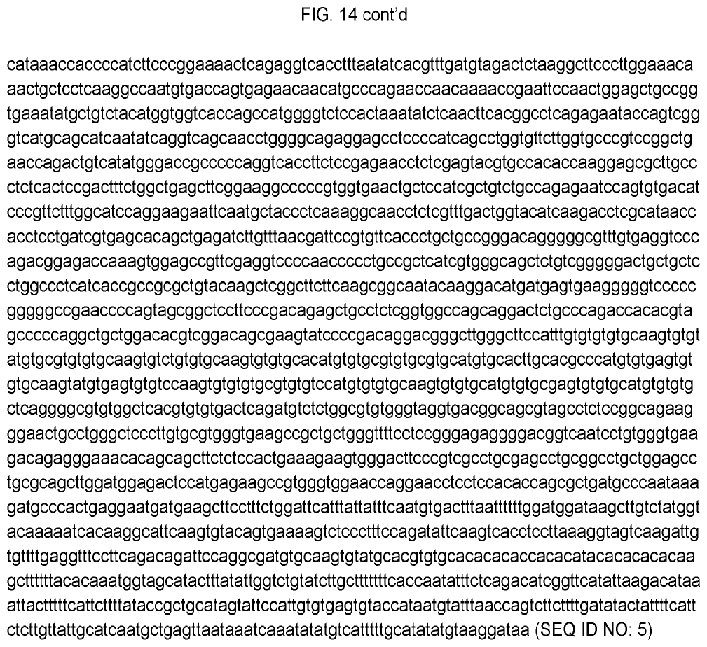

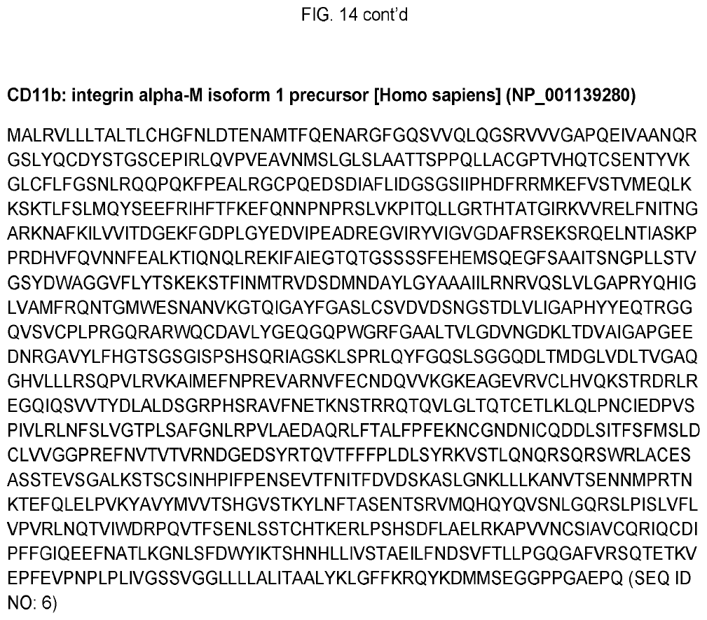

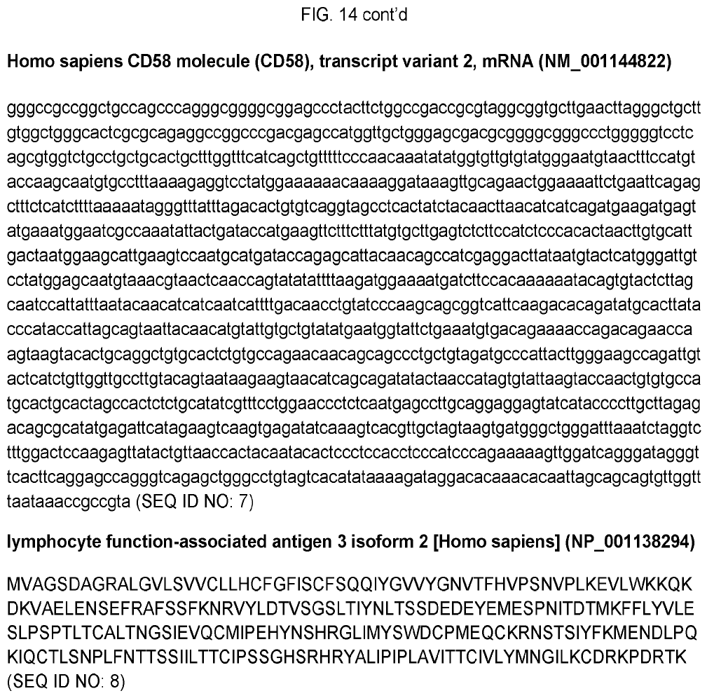

FIG. 10. Validation of combination selectivity between AML and CLL. (10A) The difference of median IC₅₀ CR values (AML-CLL) was computed for each of 48 indicated\ combinations using the Hodges-Lehmann method. The median difference is represented by a closed circle, and the 95% confidence interval is shown as the colored bar. AML-selective and CLL-selective combinations are colored orange or green, respectively. (10B) Spearman correlation of log-transformed median IC₅₀ CR and AUC CR values between the discovery sample cohort (n=122) and independent validation cohort (n=151). (10C) Validation of select effective combinations within AML or CLL diagnostic subgroups. Scatter plots of log-transformed IC₅₀ CR values for the indicated combinations. Black horizontal bars represent median CR; FDR-adjusted p-values (Wilcoxon Sign Rank test of median) are shown.

Although several links between actionable diagnostic and genetic features and effective combinations of target agents are disclosed, certain limitations to the analyses exist. For example, the disclosed screening methods require the use of prospectively collected, freshly isolated patient samples as a consequence of a clinical visit, and thus restricts the ability to perform multiple independent tests on a given time point for a patient. Furthermore, variability in the number of mononuclear cells recovered from a sample limits the scope and number of inhibitors that may be tested. Collectively, these limitations present the challenge of overcoming noise in the drug sensitivity data due to variations in biological response and in technique, and as such limit the utility of applying conventional methods of determining synergy, such as those described by Chou and Talalay (Chou, 2006 supra). Additionally, due to the prospective collection nature of sample inclusion, there are differences in sample size and frequency of genetic parameters surveyed within a given diagnostic group. To address these issues, an integrated approach was used. This approach included the use of a Combination Ratio as a measure of combination effectiveness, requiring agreement between two effect measures (IC₅₀ and AUC) for significance, excluding effect measures from analysis if their fit to the probit regression model was poor, and performing rank-based tests of the median both within and between relevant subgroups for contextual interpretation. Finally, the disclosed assay makes use of aggregate readings of whole mononuclear cell population responses to drug combinations and single-agents, where a readout that offers granularity of responses at the single-cell level would provide additional data for parsing of drug combination efficacy. Recent approaches that make use of single-cell imaging or flow cytometry (Irish J M et al, Cell 118, 217-228 (2004); Kornblau S M et al, Clin Cancer Res 16, 3721-3733 (2010); Del Gaizo Moore V and Letai A, Cancer Lett 332, 202-205 (2013); and Touzeau C et al, Leukemia 30, 761-764 (2016); all of which are incorporated by reference herein) will be useful to enhancing the initial view of combination efficacy.

In sum, the disclosed data identify effective drug combinations that were previously unrecognized and might promote the testing of certain of these drug combinations in clinical trials. Collectively, this may yield new therapeutic options for patients while advancing the use of ex vivo functional testing as a valid assay in the clinical decision-making process.

Patient samples: All patients were consented for participation in this study with the approval and guidance of the Internal Review Boards at Oregon Health & Science University, University of Utah, University of Texas Southwestern, Stanford University, University of Miami, and University of Colorado. Mononuclear cells were isolated by Ficoll-gradient centrifugation from freshly obtained bone marrow aspirates or peripheral blood draws and plated into assays within 24 hours. All samples were analyzed for clinical characteristics and drug sensitivity. AML and CLL patient samples were additionally analyzed with respect to expanded, disease-specific panels of clinical, prognostic, genetic, cytogenetic, and surface antigen characteristics. Genetic characterization of AML samples included mining of a clinical deep sequencing panel of genes commonly mutated in hematologic malignancies (GeneTrails panel from the Knight Diagnostic Laboratories, OHSU).

Ex vivo functional screen: Small molecule inhibitors, purchased from LC Laboratories (Woburn, Mass., USA) and Selleck Chemicals (Houston, Tex., USA), were reconstituted in DMSO and stored at −80° C. The CSF1R inhibitor, ARRY-382 was obtained from Array Biopharma, Inc. Inhibitors were initially distributed into 384-well master plates, from which destination plates prepared with a single agent/well in a 7-point concentration series ranging from 10 μM to 0.0137 μM for each drug except dasatinib, which was plated using a concentration range of 1 μM to 0.00137 μM. Destination plates prepared with a single agent/well contain a concentration series ranging from 10 μM to 0.01 μM for each drug except dasatinib, which has a concentration range of 1 μM to 0.001 μM. Similar destination plates were prepared with the 48 indicated pair-wise inhibitor combinations in identical 7-point fixed molar concentration series to those used for single agents. The final concentration of DMSO was ≤0.1% in all wells, and all sets of single agent and combination destination plates were stored at −20° C. and thawed just prior to use. Primary mononuclear cells were plated across single agent and combination inhibitor panels within 24 h of collection. Cells were seeded into 384 well assay plates at 10,000 cells/well in RPMI-1640 media supplemented with fetal bovine serum (10%), L-glutamine, penicillin-streptomycin and β-mercaptoethanol (10⁻⁴ M). After three days of culture at 37° C., 5% CO₂, methanethiosulfonate (MTS) reagent (CellTiter96 AQₛᵤₑₒᵤₛ One; Promega Madison, Wis., USA) was added and optical density was measured at 490 nm and used to determine cell viability (normalized to untreated control wells).

Inhibitor dose-response curve analysis and effect measure calculations: Normalized absorbance values at each dose of a 7-point dilution series for 21 small-molecule inhibitors and 48 pair-wise combinations of two of these single agents were analyzed for each of 122 primary leukemia samples. Dose concentrations were log-transformed and a probit regression curve was fit to each 7-point drug sensitivity profile using maximum likelihood estimation for the intercept and slope. This parametric model was chosen over a polynomial because the probit's monotonic inverse-sigmoidal shape reflects a dose-response curve typically seen in samples incubated with cytotoxic or inhibitory agents. Normalized viability values greater than 100%, indicating higher cell viability than the average viability across control wells on the given plate, were truncated to 100% to produce a percentage response variable amenable to probit modeling. Each profile's IC₅₀, defined as the lowest dose to achieve a reduction to 50% viability, and area under the curve (AUC) were programmatically derived from the fitted probit curve and confined within the limits of the tested dose range. To quantify the efficacy of an equimolar drug combination in comparison to its constituent single agents, a Combination Ratio (CR) effect measure was generated based on each of the IC₅₀ and AUC values for each inhibitor triad (the drug combination and the two single agents). The IC₅₀ CR and AUC CR values were defined to be the ratio of the combination drug's IC₅₀ or AUC to the minimum IC₅₀ or AUC for the two single agents, respectively. Each sensitivity profile modeled by probit regression was assigned a fit statistic based on the p-value for the test of whether the fitted curve's slope was horizontal. Generally, a smaller fit statistic produced by a decreasing slope indicates a better fitting probit model and, by extension, provides a measure of confidence in the curve-derived IC₅₀ and AUC for a particular sample/drug pairing. An effect measure value less than 1 indicates that a sample is more sensitive to the equimolar drug combination than to either of the single agents alone.

Statistical analysis: Unsupervised hierarchical clustering and heatmap displays of inhibitor sensitivity were generated using GenePattern software (Broad Institute). Inhibitor combination efficacy was compared for all samples (n=122) across a panel of general clinical variables: diagnostic category (AML, ALL, CLL, MPN or MDS/MPN), age (categorized as <18, 18-39, 40-64, ≥65), sex, and type of specimen (peripheral blood, bone marrow aspirate, leukapheresis). For each effect measure, a one-sample Wilcoxon Signed-Rank (WSR) test was used to assess whether the median CR value within a particular categorical subgroup, if less than 1, was statistically significant. Furthermore, differences in median CR across the subgroups of these variables were evaluated with the Kruskal-Wallis test. Additional diagnosis-specific clinical and genetic variables were examined for AML (n=58) and CLL (n=42) patient samples. Similar tests were performed on a subgroup's median CR and on differences across subgroups for each of these clinical or genetic variables. Subgroups for all mutations, cytogenetic abnormalities, and cell surface antigens were defined as either positive or negative. Correlations between continuous clinical variables (e.g. WBC count) and CR values were appraised with Spearman p-values. For all within-subgroup and between-subgroup non-parametric tests, false discovery rate (FDR) adjustments (Benjamini Y and Hochberg Y, J Ed Behav Stat 25, 60-83 (2000); incorporated by reference herein) were applied to p-values with an adaptive linear step-up method to account for tests being performed on each of the 48 inhibitor triads.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the efficacy of the drug combination at issue according to a test of efficacy disclosed herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004.

1. A method of identifying a subject with JQ1/palbociclib-responsive acute myeloid leukemia comprising:
obtaining a biological sample from a living human patient with acute myeloid leukemia;
detecting whether a DNMT3A mutation is present or absent in the biological sample by contacting the biological sample with a probe that binds to DNMT3A; and
detecting binding between the probe and the DNMT3A,
wherein the subject is identified as having JQ1/palbociclib-responsive acute myeloid leukemia if mutated DNMT3A is detected in the sample based on the binding of the probe.

2. A method of claim 1 wherein the biological sample comprises: mononuclear cells, a peripheral blood sample, or a bone marrow aspirate.

3. A method of claim 1 wherein the mutated DNMT3A comprises a DNMT3A mutated at protein position 882.

4. A method of claim 1 wherein the DNMT3A mutated at protein position 882 comprises R882H.

5. A method of claim 1 wherein the probe comprises an allele specific primer, and wherein the detecting binding between the probe and the DNMT3A comprises performing allele specific PCR.

6. A method of claim 1 wherein the probe comprises a 25-mer to 75-mer oligonucleotide probe and wherein the detecting binding between the probe and the DNMT3A comprises performing an oligonucleotide microarray assay.

7. A method of identifying a subject with JQ1/palbociclib-responsive acute myeloid leukemia and treating the subject comprising:
obtaining a biological sample from a living human patient with acute myeloid leukemia;
detecting whether mutated DNMT3A is present or absent in the biological sample;
identifying the subject as having JQ1/palbociclib-responsive acute myeloid leukemia when mutated DNMT3A is detected in the biological sample; and
administering an effective amount of JQ1 and palbociclib to the subject.

8. A method of claim 7 wherein the biological sample comprises: mononuclear cells, a peripheral blood sample, or a bone marrow aspirate.

9. A method of claim 7 wherein the mutated DNMT3A comprises a DNMT3A mutation at protein position 882.

10. A method of claim 7 wherein the DNMT3A mutation at protein position 882 comprises R882H.

11. A method of claim 7 wherein the detecting whether mutated DNMT3A is present or absent comprises contacting the sample with a probe that binds to DNMT3A.

12. A method of claim 7 wherein the probe comprises an allele specific primer, and wherein the contacting the sample with a probe that binds to DNMT3A comprises performing allele specific PCR.

13. A method of claim 7 wherein the probe comprises a 25-mer to 75-mer oligonucleotide probe and wherein the contacting the sample with a probe that binds to DNMT3A comprises performing an oligonucleotide microarray assay.

14. A method of claim 7 wherein the JQ1 and palbociclib are co-formulated.

15. A method of claim 7 wherein JQ1 and palbociclib are administered at a dose equivalent to the combined IC₅₀, IC₇₀, or IC₉₀ of JQ1 and palbociclib.

16. A method of claim 7 wherein the JQ1 is administered at a dose between 20 mg/day and 800 mg/day, and wherein the palbociclib is administered at a dose of between 1 mg/day and 200 mg/day.

17. A method of providing a targeted combination therapy to a human patient in need thereof comprising:
identifying a human patient with acute myeloid leukemia and a DNMT3A mutation; and
administering a therapeutically effective amount of JQ1 and a CDK4/6 inhibitor to the human patient,
thereby providing a targeted combination therapy to the human patient, wherein the CDK4/6 inhibitor is palbociclib.

18. A method of claim 17 wherein the DNMT3A mutation comprises a DNMT3A mutation at protein position 882.

19. A method of claim 17 wherein the DNMT3A mutation at protein position 882 comprises R882H.

20. A method of claim 17 wherein the JQ1 and palbociclib are co-formulated.

21. A method of claim 17 wherein JQ1 and palbociclib are administered at a dose equivalent to the combined IC₅₀, IC₇₀, or IC₉₀ of JQ1 and palbociclib.

22. A method of claim 17 wherein the identifying a human patient with a DNMT3A mutation comprises sequencing a DNMT3A gene in the sample and comparing the sequence to wild-type DNMT3A, wherein a difference between the sequence of the DNMT3A in the sample and the sequence of wild-type DNMT3A indicates a DNMT3A mutation.

23. A method of claim 17 wherein the DNMT3A mutation comprises a DNMT3A mutation at protein position 882, and wherein the identifying a human patient with a DNMT3A mutation at R882 comprises performing a microarray assay with one or more probes specific to wild-type DNMT3A R882 or one or more probes specific to DNMT3A R882H.

24. A method of claim 17 wherein the JQ1 is administered at a dose between 20 mg/day and 800 mg/day, and wherein the palbociclib is administered at a dose of between 1 mg/day and 200 mg/day.

25. A method of identifying a subject with JQ1/sorafenib-responsive acute myeloid leukemia comprising:
obtaining a biological sample from a living human patient with acute myeloid leukemia;
detecting whether NPM1 is mutated in the biological sample by contacting the biological sample with a probe that binds to NPM1;
detecting binding between the probe and the mutated NPM1,
wherein the subject is identified as having JQ1/sorafenib-responsive acute myeloid leukemia if mutated NPM1 is detected in the sample based on the binding between the probe and the NPM1.

26. A method of claim 25 wherein the biological sample comprises: mononuclear cells, a peripheral blood sample, or a bone marrow aspirate.

27. A method of claim 25 wherein the mutated NPM1 comprises a NPM1 with a mutation in exon 12.

28. A method of claim 25 wherein the mutation in exon 12 comprises 1-4 base pairs, inserted directly following gene position 950, 951, 952, 953, 954, 955, 956, 957, 958, 959, 960, 961, 962, 963, 964, 965, 966, 967, 968, 969, or 970.

29. A method of claim 25 wherein the insertion comprises four base pairs, inserted directly following gene position 960.

30. A method of claim 25 wherein the four base pairs comprise TCTG.

31. A method of claim 25 wherein the probe comprises a primers, and wherein the detecting binding between the probe and the NPM1 comprises amplifying NPM1 by PCR and sequencing the amplicons.

32. A method of claim 25 wherein the probe comprises a 25-mer to 75-mer oligonucleotide probe and wherein the detecting binding between the probes and the NPM1 comprises performing an oligonucleotide microarray assay.

33. A method of identifying a subject with JQ1/sorafenib-responsive acute myeloid leukemia and treating the subject comprising:
obtaining a biological sample from a living human patient with acute myeloid leukemia;
detecting whether mutated NPM1 is present or absent in the biological sample;
identifying the subject as having JQ1/sorafenib-responsive acute myeloid leukemia when mutated NPM1 is detected in the biological sample; and
administering an effective amount of JQ1 and sorafenib to the subject.

34. A method of claim 33 wherein the biological sample comprises: mononuclear cells, a peripheral blood sample, or a bone marrow aspirate.

35. A method of claim 33 wherein the mutated NPM1 comprises a NPM1 with a mutation in exon 12.

36. A method of claim 33 wherein the mutation in exon 12 comprises 1-4 base pairs, inserted directly following gene position 950, 951, 952, 953, 954, 955, 956, 957, 958, 959, 960, 961, 962, 963, 964, 965, 966, 967, 968, 969, or 970.

37. A method of claim 33 wherein the insertion comprises four base pairs, inserted directly following gene position 960.

38. A method of claim 33 wherein the detecting whether mutated NPM1 is present or absent comprises contacting the sample with a probe that binds to NPM1.

39. A method of claim 38 wherein the probe comprises a primer, and wherein the detecting binding between the probe and the NPM1 comprises amplifying the NPM1 by PCR and sequencing the amplicons.

40. A method of claim 38 wherein the probes comprise 25-mer to 75-mer oligonucleotide probes and wherein the detecting binding between the probes and the mutated NPM1 comprises performing an oligonucleotide microarray assay.

41. A method of claim 33 wherein the JQ1 and sorafenib are co-formulated.

42. A method of claim 33 wherein JQ1 and sorafenib are administered at a dose equivalent to the combined IC₅₀, IC₇₀, or IC₉₀ of JQ1 and sorafenib.

43. A method of claim 33 wherein the JQ1 is administered at a dose between 20 mg/day and 800 mg/day, and wherein the sorafenib is administered at a dose of between 50 mg/day and 1200 mg/day.

44. A method of providing a targeted combination therapy to a human patient in need thereof comprising:
identifying a human patient with acute myeloid leukemia and a NPM1 mutation; and
administering a therapeutically effective amount of JQ1 and a tyrosine kinase inhibitor to the human patient,
thereby providing a targeted combination therapy to the human patient, wherein the tyrosine kinase inhibitor is sorafenib.

45. A method of claim 44 wherein the mutated NPM1 comprises a NPM1 with a mutation in exon 12.

46. A method of claim 44 wherein the mutation in exon 12 comprises 1-4 base pairs, inserted directly following gene position 950, 951, 952, 953, 954, 955, 956, 957, 958, 959, 960, 961, 962, 963, 964, 965, 966, 967, 968, 969, or 970.

47. A method of claim 44 wherein the insertion comprises four base pairs, inserted directly following gene position 960.

48. A method of claim 44 wherein the JQ1 and sorafenib are co-formulated.

49. A method of claim 44 wherein JQ1 and sorafenib are administered at a dose equivalent to the combined IC₅₀, IC₇₀, or IC₉₀ of JQ1 and sorafenib.

50. A method of claim 44 wherein the identifying a human patient with a NPM1 mutation in exon 12 comprises performing PCR to amplify exon 12 and sequencing the amplicons.

51. A method of identifying a subject with ruxolitinib/cabozanatinib-responsive acute myeloid leukemia comprising:
obtaining a biological sample from a living human patient with acute myeloid leukemia; and
detecting whether a normal karyotype is present or absent in the biological sample by contacting the sample with one or more dyes that stain chromosomes and visualizing the chromosomes to detect the karyotype of the sample;
wherein the subject is identified as having ruxolitinib/cabozanatinib-responsive acute myeloid leukemia if a normal karyotype is detected in the sample.

52. A method of claim 51 wherein the biological sample comprises: mononuclear cells, a peripheral blood sample, or a bone marrow aspirate.

53. A method of claim 51 wherein the normal karyotype comprises less than three chromosomal aberrations.

54. A method of claim 51 wherein the normal karyotype comprises no chromosomal aberrations.

55. A method of claim 51 wherein the dye comprises orcein or giesma.

56. A method of identifying a subject with ruxolitinib/cabozanatinib-responsive acute myeloid leukemia and treating the subject comprising:
obtaining a biological sample from a living human patient with acute myeloid leukemia;
detecting whether a normal karyotype is present or absent in the biological sample;
identifying the subject as having ruxolitinib/cabozanatinib-responsive acute myeloid leukemia when a normal karyotype is detected in the biological sample; and
administering an effective amount of ruxolitinib and cabozanatinib to the subject.

57. A method of claim 56 wherein the biological sample comprises: mononuclear cells, a peripheral blood sample, or a bone marrow aspirate.

58. A method of claim 56 wherein the normal karyotype comprises less than three chromosomal aberrations.

59. A method of claim 56 wherein the normal karyotype comprises no chromosomal aberrations.

60. A method of claim 56 wherein the ruxolitinib and cabozanatinib are co-formulated.

61. A method of claim 56 wherein ruxolitinib and cabozanatinib are administered at a dose equivalent to the combined IC50, IC70, or IC90 of ruxolitinib and cabozanatinib.

62. A method of claim 56 wherein the ruxolitinib is administered at a dose between 1 mg/day and 200 mg/day, and wherein the cabozanatinib is administered at a dose of between 1 mg/day and 200 mg/day.

63. A method of claim 56 wherein the detecting whether a normal karyotype is present or absent in the biological sample comprises performing standard chromosome analysis.

64. A method of claim 56 wherein the detecting whether a normal karyotype is present or absent in the biological sample comprises contacting the sample with one or more dyes that stain chromosomes, and visualizing the chromosomes by microscopy to identify chromosomal aberrations, wherein an absence of chromosomal aberrations indicates a normal karyotype.

65. A method of claim 64 wherein the one or more dyes comprise orcein or giesma.

66. A method of providing a targeted combination therapy to a human patient in need thereof comprising:
identifying a human patient with acute myeloid leukemia and a normal karyotype; and
administering a therapeutically effective amount of cabozanatinib and a tyrosine kinase inhibitor to the human patient,
thereby providing a targeted combination therapy to the human patient, wherein the tyrosine kinase inhibitor is ruxolitinib.

67. A method of claim 66 wherein the ruxolitinib and cabozanatinib are co-formulated.

68. A method of claim 66 wherein ruxolitinib and cabozanatinib are administered at a dose equivalent to the combined IC50, IC70, or IC90 of ruxolitinib and cabozanatinib.

69. A method of claim 66 wherein the identifying a human patient with a normal karyotype comprises performing standard chromosome analysis.

70. A method of claim 66 wherein the identifying a human patient with a normal karyotype comprises contacting a sample from the human patient with one or more dyes that stain chromosomes, and visualizing the chromosomes by microscopy to identify chromosomal aberrations, wherein an absence of chromosomal aberrations indicates a normal karyotype.

71. A method of claim 70 wherein the one or more dyes comprise orcein or giesma.

72. A method of claim 66 wherein the normal karyotype comprises less than three chromosomal aberrations.

73. A method of claim 66 wherein the normal karyotype comprises no chromosomal aberrations.

74. A method of claim 66 wherein the ruxolitinib is administered at a dose between 1 mg/day and 200 mg/day, and wherein the cabozanatinib is administered at a dose of between 1 mg/day and 200 mg/day.

75. A method of identifying a subject with idelalisib/quizartinib-responsive acute myeloid leukemia comprising:
obtaining a biological sample from a living human patient with acute myeloid leukemia; and
detecting whether the biological sample is positive for complex karyotype by contacting the sample with one or more dyes that bind to chromosomes and analyzing the chromosomes by microscopy to identify chromosomal aberrations,
wherein the subject is identified as having idelalisib/quizartinib-responsive acute myeloid leukemia if the sample is identified as positive for complex karyotype.

76. A method of claim 75 wherein the biological sample comprises: mononuclear cells, a peripheral blood sample, or a bone marrow aspirate.

77. A method of claim 75 wherein the sample is identified as positive for complex karyotype if (i) three or more chromosomal aberrations are present, and (ii) the absence of: a translocation comprising t(8;21)(q22;q22), an inversion comprising inv(16)(p13q22)/t(16;16)(p13;q22) and a translocation comprising t(15;17)(q22;q21).

78. A method of claim 75 wherein the chromosomal aberrations comprise at least one of: monosomies, deletions or unbalanced translocations.

79. A method of claim 75 wherein the one or more dyes comprise orcein or giesma.

80. A method of identifying a subject with idelalisib/quizartinib-responsive acute myeloid leukemia and treating the subject comprising:
obtaining a biological sample from a living human patient with acute myeloid leukemia;
detecting whether a complex karyotype is present or absent in the biological sample;
identifying the subject as having idelalisib/quizartinib-responsive acute myeloid leukemia when a complex karyotype is detected in the biological sample; and
administering an effective amount of idelalisib and quizartinib to the subject.

81. A method of claim 80 wherein the biological sample comprises: mononuclear cells, a peripheral blood sample, or a bone marrow aspirate.

82. A method of claim 80 wherein the detecting whether a complex karyotype is present comprises performing standard chromosome analysis.

83. A method of claim 80 wherein the detecting whether a complex karyotype is present or absent in the biological sample comprises contacting the sample with one or more dyes that bind to chromosomes and analyzing the chromosomes by microscopy to identify chromosomal aberrations.

84. A method of claim 83 wherein the one or more dyes comprise orcein or giesma.

85. A method of claim 80 wherein the detecting whether a complex karyotype is present comprises performing FISH or a chromosomal microarray.

86. A method of claim 80 wherein the complex karyotype comprises (i) the presence of three or more chromosomal aberrations are present, and (ii) the absence of t(8;21)(q22;q22), inv(16)(p13q22)/t(16;16)(p13;q22) and t(15;17)(q22;q21).

87. A method of claim 80 wherein the chromosomal aberrations comprise at least one of: monosomies, deletions or unbalanced translocations.

88. A method of claim 80 wherein idelalisib and quizartinib are co-formulated.

89. A method of claim 80 wherein idelalisib and quizartinib are administered at a dose equivalent to the combined IC50, IC70, or IC90 of idelalisib and quizartinib.

90. A method of claim 80 wherein the idelalisib is administered at a dose between 20 mg/day and 800 mg/day, and wherein the quizartinib is administered at a dose of between 50 mg/day and 1200 mg/day.

91. A method of providing a targeted combination therapy to a human patient in need thereof comprising:
identifying a human patient with acute myeloid leukemia and a complex karyotype; and
administering a therapeutically effective amount of a PI3 kinase inhibitor and a FLT3/CSF1R inhibitor to the human patient,
thereby providing a targeted combination therapy to the human patient, wherein the PI3 kinase inhibitor is idelalasib and the FLT3/CSF1R inhibitor is quizartinib.

92. A method of claim 91 wherein the idelalisib and quizartinib are co-formulated.

93. A method of claim 91 wherein idelalisib and quizartinib are administered at a dose equivalent to the combined IC50, IC70, or IC90 of idelalisib and quizartinib.

94. A method of claim 91 wherein identifying a patient with a complex karyotype comprises performing standard chromosome analysis on a sample from the human patient.

95. A method of claim 91 wherein identifying a patient with a complex karyotype comprises performing FISH or a chromosomal microarray on a sample from the human patient.

96. A method of claim 91 wherein the complex karyotype comprises (i) the presence of three or more chromosomal aberrations, and (ii) the absence of: a translocation comprising t(8;21)(q22;q22), an inversion comprising inv(16)(p13q22)/t(16;16)(p13;q22) and a translocation comprising t(15;17)(q22;q21).

97. A method of claim 91 wherein the chromosomal aberrations comprise at least one of: monosomies, deletions or unbalanced translocations.

98. A method of claim 91 wherein the idelalisib is administered at a dose between 20 mg/day and 800 mg/day, and wherein the quizartinib is administered at a dose of between 50 mg/day and 1200 mg/day.

99. A method of identifying a subject with venetoclax/JQ1-responsive acute myeloid leukemia comprising:
obtaining a biological sample from a living human patient with acute myeloid leukemia;
detecting the presence or absence of CD11b in the biological sample by contacting the sample with an anti-CD11b antibody or a CD11b probe and detecting binding between the antibody or probe and CD11b,
wherein the subject is identified as having venetoclax/JQ1-responsive acute myeloid leukemia if CD11b is present in the sample.

100. A method of claim 99 wherein the biological sample comprises: mononuclear cells, a peripheral blood sample, or a bone marrow aspirate.

101. A method of claim 99 wherein the detecting the presence or absence of CD11b in the biological sample comprises contacting the sample with an anti-CD11b antibody.

102. A method of claim 101 wherein detecting the presence or absence of CD11b comprises performing flow cytometry.

103. A method of identifying a subject with venetoclax/JQ1-responsive acute myeloid leukemia and treating the subject comprising:
obtaining a biological sample from a living human patient with acute myeloid leukemia;
detecting whether CD11b is expressed in the biological sample;
identifying the subject as having venetoclax/JQ1-responsive acute myeloid leukemia when CD11b is detected in the biological sample; and
administering an effective amount of venetoclax and JQ1 to the subject.

104. A method of claim 103 wherein the biological sample comprises: mononuclear cells, a peripheral blood sample, or a bone marrow aspirate.

105. A method of claim 103 wherein identifying whether CD11b is expressed comprises performing flow cytometry.

106. A method of claim 103 wherein the venetoclax and JQ1 are co-formulated.

107. A method of claim 103 wherein venetoclax and JQ1 are administered at a dose equivalent to the combined IC50, IC70, or IC90 of venetoclax and JQ1.

108. A method of claim 103 wherein the venetoclax is administered at a dose between 5 mg/day and 600 mg/day, and wherein the JQ1 is administered at a dose of between 20 mg/day and 800 mg/day.

109. A method of providing a targeted combination therapy to a human patient in need thereof comprising:
identifying a human patient who has acute myeloid leukemia and is positive for CD11b expression; and
administering a therapeutically effective amount of a Bcl2 inhibitor and JQ1 to the human patient,
thereby providing a targeted combination therapy to the human patient, wherein the Bcl2 inhibitor is venetoclax.

110. A method of claim 109 wherein the venetoclax and JQ1 are co-formulated.

111. A method of claim 109 wherein venetoclax and JQ1nib are administered at a dose equivalent to the combined IC50, IC70, or IC90 of venetoclax and JQ1.

112. A method of claim 109 wherein the identifying a human patient who is positive for CD11b expression comprises performing flow cytometry on a biological sample from the patient and detecting the presence or absence of CD11b in the sample, wherein the presence of CD11b in the sample indicates that the human patient is positive for CD11b.

113. A method of claim 109 wherein the venetoclax is administered at a dose between 5 mg/day and 600 mg/day, and wherein the JQ1 is administered at a dose of between 20 mg/day and 800 mg/day.

114. A method of identifying a subject with venetoclax/doramapimod-responsive acute myeloid leukemia comprising:
obtaining a biological sample from a human patient with acute myeloid leukemia; and
detecting the presence or absence of CD58 in the biological sample by contacting the sample with an anti-CD58 antibody or a CD58 probe and detecting binding between the anti-CD58 antibody or a CD58 probe and CD58,
wherein the patient is identified as having venetoclax/doramapimod-responsive acute myeloid leukemia if CD58 is present in the sample.

115. A method of claim 114 wherein the biological sample comprises: mononuclear cells, a peripheral blood sample, or a bone marrow aspirate.

116. A method of claim 114 wherein the detecting the presence or absence of CD58 in the biological sample comprises contacting the sample with an anti-CD58 antibody.

117. A method of claim 114 wherein detecting the presence or absence of CD58 comprises performing flow cytometry.

118. A method of identifying a subject with venetoclax/doramapimod-responsive acute myeloid leukemia and treating the subject comprising:
obtaining a biological sample from a human patient with acute myeloid leukemia;
detecting whether CD58 is expressed in the biological sample;
identifying the patient as having venetoclax/doramapimod-responsive acute myeloid leukemia when CD58 is detected in the biological sample; and
administering an effective amount of venetoclax and doramapimod to the subject.

119. A method of claim 118 wherein the biological sample comprises: mononuclear cells, a peripheral blood sample, or a bone marrow aspirate.

120. A method of claim 118 wherein the detecting whether CD58 is expressed comprises performing flow cytometry with an anti-CD58 antibody.

121. A method of claim 118 wherein the venetoclax and doramapimod are co-formulated.

122. A method of claim 118 wherein venetoclax and doramapimod are administered at a dose equivalent to the combined IC50, IC70, or IC90 of venetoclax and doramapimod.

123. A method of claim 118 wherein the venetoclax is administered at a dose between 5 mg/day and 600 mg/day, and wherein the doramapimod is administered at a dose of between 1 mg/day and 600 mg/day.

124. A method of providing a targeted combination therapy to a human patient in need thereof comprising:
identifying a human patient with acute myeloid leukemia and CD58 expression; and
administering a therapeutically effective amount of a Bcl2 inhibitor and doramapimod to the human patient,
thereby providing a targeted combination therapy to the human patient, wherein the Bcl2 inhibitor is venetoclax.

125. A method of claim 124 wherein the venetoclax and doramapimod are co-formulated.

126. A method of claim 124 wherein venetoclax and doramapimod are administered at a dose equivalent to the combined IC50, IC70, or IC90 of venetoclax and doramapimod.

127. A method of claim 124 wherein the venetoclax is administered at a dose between 5 mg/day and 600 mg/day, and wherein the doramapimod is administered at a dose of between 1 mg/day and 600 mg/day.

128. A method of claim 124 wherein the identifying a human patient who is positive for CD58 expression comprises performing flow cytometry on a biological sample from the patient and detecting the presence or absence of CD58 in the sample, wherein the presence of CD58 in the sample indicates that the human patient is positive for CD58.

129. A method of identifying a subject with chronic lymphoblastic leukemia that is responsive to combined therapy with (i) palbociclib and (ii) venetoclax or trametinib comprising:
obtaining a biological sample from a human patient with chronic lymphoblastic leukemia;
detecting whether there is a chromosome 13q deletion in the biological sample by contacting the sample with (i) a labelled probe that bind to chromosome 13q or (ii) a dye that stains chromosomes for microscopy analysis; and
detecting binding between the probes or the dye and chromosome 13q,
wherein the subject is identified as having chronic lymphoblastic leukemia that is responsive to combined therapy with (i) palbociclib and (ii) venetoclax or trametinib if a chromosome 13q deletion is detected in the sample.

130. A method of claim 129 wherein the biological sample comprises: mononuclear cells, a peripheral blood sample, or a bone marrow aspirate.

131. A method of claim 129 wherein the detecting whether there is a chromosome 13q deletion in the biological sample comprises contacting the sample with a dye that stains chromosomes for microscopy analysis, and wherein the dye comprises orcein or giesma.

132. A method of claim 129 wherein the detecting whether there is a chromosome 13q deletion in the biological sample comprises contacting the sample with a labelled probe that binds to chromosome 13q, and performing FISH or a chromosomal microarray.

133. A method of (a) identifying a subject with chronic lymphoblastic leukemia that is responsive to combined therapy with (i) palbociclib and (ii) venetoclax or trametinib and (b) treating the subject comprising:
obtaining a biological sample from a living human patient with chronic lymphoblastic leukemia;
detecting whether a chromosome 13q deletion is present in the biological sample;
identifying the subject as having chronic lymphoblastic leukemia that is responsive to combined therapy with (i) palbociclib and (ii) venetoclax or trametinib when a chromosome 13q deletion is detected in the biological sample; and
administering an effective amount of (i) palbociclib and (ii) venetoclax or trametinib to the subject.

134. A method of claim 133 wherein the biological sample comprises: mononuclear cells, a peripheral blood sample, or a bone marrow aspirate.

135. A method of claim 133 wherein the detecting whether a chromosome 13q deletion is present in the biological sample comprises contacting the sample with a dye that stains chromosomes for microscopy analysis.

136. A method of claim 133 wherein the detecting whether a chromosome 13q deletion is present comprises performing FISH or a chromosomal microarray.

137. A method of claim 133 wherein the (i) palbociclib and (ii) venetoclax or trametinib are co-formulated.

138. A method of claim 133 wherein palbociclib and venetoclax are administered at a dose equivalent to the combined IC50, IC70, or IC90 of palbociclib and venetoclax.

139. A method of claim 133 wherein palbociclib and trametinib are administered at a dose equivalent to the combined IC50, IC70, or IC90 of palbociclib and trametinib.

140. A method of claim 133 wherein the palbociclib is administered at a dose between 1 mg/day and 200 mg/day, and wherein the venetoclax is administered at a dose of between 5 mg/day and 600 mg/day.

141. A method of claim 133 wherein the palbociclib is administered at a dose between 1 mg/day and 200 mg/day, and wherein the trametinib is administered at a dose of between 0.1 mg/day and 5 mg/day.

142. A method of providing a targeted combination therapy to a human patient in need thereof comprising:
identifying a human patient with chronic lymphoblastic leukemia and a chromosome 13q deletion; and
administering a therapeutically effective amount of (i) a CDK4/6 inhibitor and (ii) a Bcl2 inhibitor or a MEK inhibitor,
thereby providing a targeted combination therapy to the human patient, wherein the CDK4/6 inhibitor is palbociclib, the Bcl2 inhibitor is venetoclax, and the MEK inhibitor trametinib.

143. A method of claim 142 wherein the (i) palbociclib and (ii) venetoclax or trametinib are co-formulated.

144. A method of claim 142 wherein palbociclib and venetoclax are administered at a dose equivalent to the combined IC50, IC70, or IC90 of palbociclib and venetoclax.

145. A method of claim 142 wherein palbociclib and trametinib are administered at a dose equivalent to the combined IC50, IC70, or IC90 of palbociclib and trametinib.

146. A method of claim 142 wherein the palbociclib is administered at a dose between 1 mg/day and 200 mg/day, and wherein the venetoclax is administered at a dose of between 5 mg/day and 600 mg/day.

147. A method of claim 142 wherein the palbociclib is administered at a dose between 1 mg/day and 200 mg/day, and wherein the trametinib is administered at a dose of between 0.1 mg/day and 5 mg/day.

148. A method of claim 142 wherein the identifying a human patient with a chromosome 13q deletion comprises performing FISH or a chromosomal microarray.

149. A method of claim 142 wherein the identifying a human patient with a chromosome 13q deletion comprises contacting the sample with a dye that stains chromosomes for microscopy analysis.
